(19) United States
(12) Patent Application Publication
(10) Pub. No.: US 2015/0337375 A1
(43) Pub. Date: Nov. 26, 2015

(54) BIOMARKERS FOR CHRONIC TRAUMATIC ENCEPHALOPATHY

(71) Applicant: The Trustees of Columbia University in the City of New York, New York, NY (US)

(72) Inventors: John Crary, Brooklyn, NY (US); Ann MCKEE, Boston, MA (US)

(21) Appl. No.: 14/652,630

(22) PCT Filed: Dec. 20, 2013
(86) PCT No.: PCT/US13/77083

§ 371 Date: Jun. 16, 2015

Related U.S. Application Data
(60) Provisional application No. 61/740,705, filed on Dec. 21, 2012.

(51) Int. Cl. C12Q 1/68; A61K 31/7105; A61K 31/713; G01N 33/68

(52) U.S. Cl. CPC C12Q 1/6883; G01N 33/6896; A61K 31/7105; A61K 31/713; C12Q 2600/156; C12Q 2600/172; G01N 2800/2814; G01N 2500/04; G01N 2333/775

(57) ABSTRACT

This invention relates to the field of screening for, identifying, diagnosing, and prognosing chronic traumatic encephalopathy (CTE). Specifically, this invention provides various biomarkers for this disease, and methods of using these biomarkers to correctly diagnose, prognose and predict those individuals who would develop CTE after suffering from mild traumatic brain injury. The invention also provides targets for drug development and basic research for CTE and preventative and therapeutic agents for CTE.

### CROSS REFERENCE TO RELATED APPLICATION

The present application claims priority to U.S. patent application Ser. No. 61/740,705 filed Dec. 21, 2012, which is hereby incorporated by reference in its entirety.

### FIELD OF THE INVENTION

This invention relates to the field of screening for, identifying, diagnosing, and prognosing chronic traumatic encephalopathy (CTE). Specifically, this invention provides various biomarkers for this disease, and methods of using these biomarkers to correctly diagnose, prognose, and predict those individuals who would develop CTE after suffering traumatic brain injury or injuries.

The invention also provides targets for drug development and basic research for CTE, and preventative and therapeutic agents for CTE.

### BACKGROUND OF THE INVENTION

The long-term neurological sequelae stemming from repetitive mild traumatic brain injury (mTBI) and traumatic brain injury (TBI) include a spectrum of progressive and debilitating neurological symptoms including affective lability, irritability, explosivity, poor attention, executive dysfunction, amnestic symptoms, suicidal ideation, parkinsonism, motor neuron disease, and dementia (Corsellis et al. (1973); Roberts et al. (1990); Gavett et al. (2011); Stern et al. (2013)). This constellation of symptoms, first described in retired professional boxers and called dementia pugilistica, is now termed chronic traumatic encephalopathy (CTE), has now been well-documented in athletes participating in other contact sports, including American football at all levels from youth to professional, professional wrestling, and ice hockey (Omalu et al. (2005); Omalu et al. (2006); McKee et al. (2009); Omalu et al. (2010)). Just this week, CTE was discovered for the first time in a professional baseball player (Tierney (2013)). Furthermore, CTE has been documented in military veterans with blast exposure, some of who received a pre-mortem diagnosis of post-traumatic stress disorder (Omalu et al. (2011(a)); Goldstein et al. (2012)). The symptoms of CTE are generally temporally separated from those stemming from the initial acute trauma, often manifesting many years after exposure. Presently, CTE can only be diagnosed at autopsy as there are no clinical biomarkers (McKee et al. (2013)).

At autopsy, the main findings in CTE are neurodegeneration and widespread axonal injury together with deposition of insoluble aggregates composed predominantly of the microtubule-associated protein tau as neurofibrillary tangles in neurons and glia (Corsellis et al. (1973); McKee et al. (2008); McKee et al. (2009); Omalu et al. (2011(b))). Many CTE patients (approximately 85%) also exhibit inclusions containing TAR DNA binding protein 43 (TDP-43), the primary disease-associated protein in sporadic amyotrophic lateral sclerosis and frontotemporal lobar degeneration (PTLD) (Neumann et al. (2006); McKee et al. (2010)).

Although the common thread linking CTE patients is repetitive mild traumatic brain injury or traumatic brain injury (mTBI or TBI), it has been proposed that genetic factors also play a role in both susceptibility and clinical course. For example, the APOE ε4 allele, a risk factor for late-onset sporadic Alzheimer's disease (AD), has been investigated in the setting of TBI (Mayuex et al. (1995); Friedman et al. (1999); DeKosky et al. (2007); Gandy et al. (2012)), but only a minority of CTE patients exhibit significant accumulation of amyloid-β (Aβ) peptide in plaques (McKee et al. (2009); Omalu et al. (2011(b))), making it unclear whether APOE is involved in CTE. Other genes, such as tau, may also be involved.

Until now there has been no way to predict whether a given individual will develop CTE as many athletes and servicemen suffer head trauma and never develop CTE. Additionally, there is no treatment for CTE. Given the millions of young Americans participating in sports, as well as a continuing active military, there is a growing need to obtain a better understanding of CTE as well as diagnostic, prevention, and treatment methods.

### SUMMARY OF THE INVENTION

The current invention is based on the surprising discovery of an increased frequency of an allele in the tau gene among individuals with documented chronic traumatic encephalopathy. This allele was found in a statistically significant number of individuals with CTE as compared to a control group. The allele was also associated with a more rapid clinical decline in those patients who had CTE. This increased allele is the H1 haplotype or allele of the tau gene (MAPT).

An association was also found between the incidence of chronic traumatic encephalopathy and an allele of the Apolipoprotein E (APOE) gene. This allele is APOE ε4.

Thus, embodiments of the current invention are tests for the presence of any one of these biomarkers in individuals who are at risk for CTE or suspected of having CTE. These individuals would be those who participate in activities where repetitive traumatic brain trauma is common, such as sports and active military duty. Perhaps a more important group of individuals for testing are those who are contemplating military service or participation in a sport. Traumatic brain injury can take place in any sport and/or recreational activity, including but not limited to, cycling, skiing, ski jumping, snowboarding, snowmobiling, bobsledding, luge, ice skating, roller blading, roller skating, inline skating, skateboarding, scooter riding, soccer, basketball, field hockey, softball, water sports (e.g., diving, scuba diving, surfing, swimming, water polo, water skiing, and water tubing), use of powered recreational vehicles (e.g., all-terrain vehicles, all-terrain cycles, dune buggies, go-carts, and mini-bikes), horseback riding, cheerleading, dancing, gymnastics, golf, trampolines, rugby, and lacrosse. Sports in which CTE has been documented include American football, boxing, ice hockey, wrestling, and baseball, both amateur and professional. CTE has also been documented in victims of physical abuse, head banging behavior, and following epileptic seizures (Gavett et al. (2011)). In particular, if such an individual tested positive for the H1 allele and/or the APOE ε4 allele, they would enter the activity with the information that they would be at higher risk for developing CTE, after TBI that would occur in these activities. These individuals could decide not to participate in these activities or at least have the knowledge that they needed to take extra precautions when participating in these activities.

Further embodiments of the current invention are methods and assays for an agent for the prevention and/or treatment of CTE. Such methods and assay would test an agent for its effect on the tau gene, and the APOE allele.

Yet another embodiment of the present invention is a prevention and/or treatment for CTE.

The Apolipoprotein E (APOE) ε4 allele frequency was found to be increased in patients with CTE. Thus, an embodiment of the present invention is a method and/or assay for screening, diagnosing, predicting and/or identifying chronic traumatic encephalopathy, comprising obtaining biological tissue and/or bodily fluid from a subject, purifying and/or isolating nucleic acid, including but not limited to cDNA and genomic DNA from the biological tissue and/or bodily fluid, and detecting the presence and/or absence of APOE ε4. Specifically, the increase of the ε4 allele in the subject would identify or diagnose the patient as having CTE or being at increased risk for CTE.

The purified and isolated nucleic acid can be obtained from any biological tissue. Preferred biological tissues include, but are not limited to, brain, and epidermis.

The purified and isolated nucleic acid can be obtained from any bodily fluid. Preferred bodily fluids include, but are not limited to, cerebrospinal fluid, whole blood, buffy coat, serum, plasma, saliva, sweat, and urine.

The nucleic acid can be purified and isolated using any method known in the art.

Detection of the APOE ε4 allele can be accomplished by any method known in the art, including, but not limited to, sequencing, hybridization with probes including Southern blot analysis and dot blot analysis, polymerase chain reaction (PCR), PCR with melting curve analysis, PCR with mass spectrometry, fluorescent in situ hybridization, DNA microarrays, single-strand conformation analysis, and restriction length polymorphism analysis.

One preferred method for the detection of the APOE ε4 allele is to amplify and sequence the Apo E gene and determine the genotype by a comparison to the known sequence for the ε4 allele.

Detection of the APOE ε4 allele can also be accomplished by allele-specific PCR. In this method, primers specific for each APOE ε4 allele are designed from the sequence of the APOE ε4 gene. These primers will anneal to the purified and isolated nucleic acid of the patient only if the particular allele is present.

Another preferred embodiment of this method and/or assay includes hybridizing the isolated and purified genomic DNA from APOE ε4 allele from the subject with probes comprising the nucleotide sequence of the APOE ε4 allele. If the probes comprising the nucleotide sequence of Apo E allele ε4 hybridizes to the isolated and purified genomic DNA from the subject, the subject is determined, diagnosed, predicted or identified as having CTE or at increased risk for CTE. In these embodiments, the isolated and purified genomic DNA or the probes must be labeled by methods known in the art for visualization if hybridization occurs.

Yet another embodiment of the present invention is a method and/or assay for screening, diagnosing, predicting and/or identifying CTE, comprising obtaining biological tissue and/or bodily fluid from a subject, purifying and/or isolating protein from said biological tissue and/or bodily fluid, and detecting the levels of Apo E ε4 protein or polypeptide in the purified and/or isolated protein sample. The level of Apo E ε4 protein or polypeptide is compared to the levels in a protein sample from a healthy control. If the levels of Apo E ε4 protein or polypeptide are different, either qualitatively, e.g., by visualization, or quantitatively, e.g., comparison to a known quantity of the protein in a healthy control, the subject can be determined, diagnosed, predicted or identified as having CTE or at increased risk for CTE. Specifically, if the level of Apo E ε4 protein or polypeptide in the protein sample from the subject is increased or higher than the level of Apo E ε4 protein or polypeptide in the protein sample from the healthy control, then the subject can be diagnosed or identified as having CTE or at increased risk for CTE.

The purified and/or isolated protein sample can be obtained from any biological tissue. Preferred biological tissues include, but are not limited to, brain, and epidermis.

The purified and/or isolated protein sample can be obtained from any bodily fluid. Preferred biological fluids include, but are not limited to, cerebrospinal fluid, whole blood, buffy coat, serum, plasma, saliva, sweat, and urine.

The protein can be obtained and processed from the biological tissue or bodily fluid by any method known in the art, in order to obtain a purified and/or isolated protein sample.

Detection of the level of Apo E ε4 protein or polypeptide can be accomplished by any method known in the art, including methods which result in qualitative results, such as ones where the existence of the protein can be visualized, either by the naked eye or by other means, and/or quantitative results. Such methods would include, but are not limited to, quantitative Western blots, immunoblots, quantitative mass spectrometry, enzyme-linked immunosorbent assays (ELISAs), radioimmunoassays (RIA), immunoradiometric assays (IRMA), and immunoenzymatic assays (IEMA) and sandwich assays using monoclonal and polyclonal antibodies.

In a preferred embodiment, the results of these methods in the subject are compared to the results of the same method in a healthy control.

In a preferred embodiment, the quantity of Apo E ε4 protein or polypeptide is measured in the protein sample from the subject and compared to a reference value of the quantity of Apo E ε4 protein or polypeptide in a healthy control, wherein the reference value represents a normal neurologic function, and finding a deviation in the quantity of Apo E ε4 protein or polypeptide from protein sample of the subject and the reference value, wherein if the quantity of Apo E ε4 protein or polypeptide from protein sample of the subject is increased or higher than the reference value, then the subject can be determined, diagnosed, predicted or identified as having CTE.

It will be understood that in addition or in the alternative, methods and/or assays that detect the APOE ε1, ε2 and/or ε3 alleles or polypeptides could also be used for screening, diagnosing, predicting and/or identifying CTE, as a subject will only have one APOE allele. Thus, if the subject has the ε1, ε2 or ε3 allele, they cannot also have the ε4 allele.

A further embodiment of the present invention is based upon the surprising findings set forth herein that the haplotype H1 of the MAPT locus is associated with CTE, a higher risk of CTE and a more severe and quicker decline from the disease.

Thus, another embodiment of the present invention is a method and/or assay for screening, diagnosing, prognosing, and/or identifying chronic traumatic encephalopathy, comprising obtaining biological tissue and/or bodily fluid from a subject, purifying and/or isolating nucleic acid, including, but not limited to, genomic DNA and RNA from the biological tissue and/or fluid, and detecting the presence of the H1 haplotype in the genomic DNA, wherein the presence of the H1 haplotype diagnoses or identifies the subject as having CTE or being at an increased risk for CTE and/or having a more rapid decline from the disease.

The purified and isolated nucleic acid can be obtained from any biological tissue. Preferred biological tissues include, but are not limited to, brain, and epidermis.

The purified and isolated nucleic acid can be obtained from any bodily fluid. Preferred bodily fluids include, but are not limited to, cerebrospinal fluid, whole blood, buffy coat, serum, plasma, saliva, sweat, and urine.

The nucleic acid can be purified and isolated using any method known in the art.

Detection of the H1 haplotype can be accomplished by any method known in the art, including, but not limited to, sequencing, hybridization with probes including Southern blot analysis and dot blot analysis, polymerase chain reaction (PCR), PCR with melting curve analysis, PCR with mass spectrometry, fluorescent in situ hybridization, DNA microarrays, and single-strand conformation analysis.

One preferred method of detection of the H1 haplotype is to amplify the MAPT locus with primers, sequence the MAPT locus and determining if the H1 haplotype is present by a comparison to known sequences of the H1 haplotype. Primers useful in this technique can be manufactured using the sequence of the MAPT locus. The MAPT H1 and H2 haplotype can be determined by PCR using the Delln9 238 by marker.

Detection of the H1 haplotype can also be accomplished by allele-specific PCR. In this method, primers specific for the H1 haplotype are designed from the sequence of the MAPT H1 haplotype. These primers will anneal to the purified and/or isolated genomic DNA of the patient only if the H1 haplotype is present.

Another preferred embodiment of this method and/or assay includes hybridizing the isolated and purified genomic DNA from MAPT locus from the subject with probes comprising the nucleotide sequence of the MAPT H1 haplotype. If the probes comprising the nucleotide sequence of MAPT H1 haplotype hybridizes to the isolated and purified genomic DNA from the subject, the subject is determined, diagnosed, predicted or identified as having CTE. In these embodiments, the isolated and purified genomic DNA or the probes must be labeled by methods known in the art for visualization if hybridization occurs.

It will be understood that in addition or in the alternative, methods and/or assays that detect the H2 haplotype could also be used for screening, diagnosing, predicting, identifying, and/or prognosing CTE, as a subject will only have one or the other haplotype. Thus, if the subject has the H2 haplotype, they cannot also have the H1 haplotype.

The present invention also includes kits embodying any of the aforementioned assays and methods.

The present invention also provides for methods and tools for drug design, testing of agents, and tools for basic research into the causes and etiology of chronic traumatic encephalopathy. The present invention also provides a method for determining target genes or proteins for drug development and basic research regarding CTE.

A further embodiment of the present invention is a method and/or assay for screening and/or identifying a test agent for the prevention and/or treatment of CTE comprising contacting or incubating a test agent to a nucleotide comprising MAPT H1 haplotype or a portion thereof, including but not limited, to regulatory elements such as the promoter, introns, exons, intron-exon junctions, 5′UTR or the 3′UTR of the H1 haplotype or the APOE ε4 allele or SEQ ID NOs: 1 or 2 or 3, and determining if the test agent binds to the nucleotide, i.e., DNA or RNA, wherein if the test agent binds to the nucleotide, the test agent is identified as a therapeutic and/or preventative agent for CTE.

A further embodiment of the present invention is a method and/or assay for screening and/or identifying a test agent for the prevention and/or treatment of CTE comprising contacting or incubating a test agent with a nucleotide comprising MAPT H1 haplotype or a portion thereof, including but not limited to, regulatory elements such as the promoter, introns, exons, intron-exon junctions, 5′UTR or the 3′UTR of the H1 haplotype or the APOE ε4 allele or SEQ ID NOs: 1 or 2 or 3, and detecting the expression of the nucleotide before and after contact or incubation with the test agent, wherein if the expression of the nucleotide is decreased after the contact or incubation with the test agent, the test agent is identified as a therapeutic and/or preventative agent for CTE.

A further embodiment of the present invention is a method and/or assay for screening and/or identifying a test agent for the prevention and/or treatment of CTE, comprising contacting or incubating a gene construct comprising a nucleotide comprising MAPT H1 haplotype or a portion thereof, including but not limited to, regulatory elements such as the promoter, introns, exons, intron-exon junctions, 5′UTR or the 3′ UTR of the H1 haplotype or the APOE ε4 allele or SEQ ID NOs: 1 or 2 or 3, and detecting the expression of the nucleotide in the gene construct before and after contacting or incubating the test agent with the gene construct, wherein if the expression of the gene is reduced or decreased after contact with the test agent or compound, the test agent is identified as a therapeutic and/or preventative agent for CTE.

A further embodiment of the present invention is a method and/or assay for screening and/or identifying a test agent for the prevention and/or treatment of CTE, comprising transforming a host cell with a gene construct comprising a nucleotide comprising MAPT H1 haplotype or a portion thereof such as regulatory elements such as the promoter, introns, exons, intron-exon junctions, 5′UTR or the 3′UTR of the H1 haplotype or the APOE ε4 allele or SEQ ID NOs: 1 or 2 or 3, detecting the expression of the nucleotide in the host cell, contacting the test agent with the host cell, and detecting the expression of the nucleotide in the host cell after contact with the test agent or compound, wherein if the expression of the nucleotide is reduced or decreased after contact with the test agent or compound, the test agent is identified as a therapeutic and/or preventative agent for CTE.

The expression of a nucleotide or gene can be determined using a measurable phenotype, either one that is native to the gene or one that is artificially linked, such as a reporter gene.

A further embodiment is a method and/or assay for screening and/or identifying a test agent for the prevention and/or treatment of CTE, comprising contacting or incubating the test agent with an Apo E ε4 polypeptide, and detecting the presence of a complex between the test agent and the polypeptide, wherein if a complex between the test agent and the polypeptide is detected, the test agent is identified as a prevention and/or treatment for CTE.

A further embodiment is a method and/or assay for screening and/or identifying a test agent for the prevention and/or treatment of CTE, comprising contacting or incubating the test agent with an Apo E ε4 polypeptide and a known ligand of the polypeptide, and detecting the presence of a complex between the test agent and the ligand, wherein if a complex between the test agent and the ligand is detected, the test agent is identified as a prevention and/or treatment for CTE.

Another embodiment of the present invention is a method and/or assay for screening and/or identifying a test agent for the prevention and/or treatment of CTE, comprising contacting or incubating the test agent with an Apo E ε4 polypeptide and a known antibody of the polypeptide, and detecting the presence and quantity of unbound antibody, wherein the presence of the unbound antibody indicates that the test agent is binding to the polypeptide, and the test agent is identified as a prevention and/or treatment for CTE.

High throughput screening can also be used to screen the test agents. Small peptides or molecules can be synthesized and bound to a surface and contacted with the polypeptides, and washed. The bound peptide is visualized and detected by methods known in the art.

Further embodiments of the present invention include methods and compositions for the treatment and/or prevention of chronic traumatic encephalopathy. One embodiment would be the treatment and/or prevention of CTE by administering an agent that binds to APOE ε4 allele, or the H1 haplotype or the H1 5′ or 3′UTR of the tau mRNA derived from the tau MAPT gene to a subject in need thereof.

A further embodiment is the administration of an agent that increases binding of a naturally occurring molecule to the APOE ε4 allele, or the H1 haplotype or the H1 3′ UTR of the tau mRNA derived from the tau MAPT gene to a subject in need thereof, either by increasing the amount or production of the molecule or by increasing binding affinity and/or stability. One such example is an miRNA that binds to the H1 3′UTR in a subject in need thereof.

As also shown the H2, haplotype of the MAPT locus is protective. Thus, a further method of treatment or prevention of CTE would be supplying the H2 haplotype to a subject in need thereof. This can be accomplished by administering a therapeutically effective amount of a composition comprising a DNA that comprises the MAPT H2 haplotype. The composition can also comprise a ligand, a conjugate, a vector, a lipid, a liposome, a carrier, an adjuvant or a diluent. The H2 haplotype of the MAPT is found at chromosome 17q21, and the H2 3′UTR is found at chromosome 17 between base pairs 76,2196-76,6698 and is set forth in SEQ ID NO: 4. The H2 promoter is set forth in SEQ ID NO: 5 (Stefansson et al. (2005))

Additionally, the APOE ε1, ε2 and ε3 alleles are not associated with disease states, thus a further method of treatment and/or prevention of CTE would be supplying these alleles to a subject in need thereof. Again this can be accomplished by administering a therapeutically effective amount of a composition comprising a DNA that comprises the APOE ε2 and/or ε3 alleles. The composition can also comprise a ligand, a conjugate, a vector, a lipid, a liposome, a carrier, an adjuvant or a diluent.

### BRIEF DESCRIPTION OF THE FIGURES

For the purpose of illustrating the invention, there are depicted in drawings certain embodiments of the invention. However, the invention is not limited to the precise arrangements and instrumentalities of the embodiments depicted in the drawings.

FIG. 1 depicts phosphorylated tau (AT8) immunostained brain sections from two Operation Enduring Freedom (OEF)/Operation Iraqi Freedom (OIF) veterans (45 (FIG. 1A) and 34 years old (FIG. 1B)) with improvised explosive device (IED) induced blast injury, and 2 young athletes (18 (FIG. 1C) and 21 years old (FIG. 1D)) with recent concussive mTBI. The frontal lobes show focal perivascular epicenters of severe tau neurofibrillary degeneration. High magnification views (FIGS. 1E-H) of the lesions demonstrate the striking perivascular tau neurofibrillary neurodegeneration. FIG. 1I is a montage illustrating the injuries found in the 45-year-old blast-injured veteran. Whole mount sections (A-D); magnification (E-F)-100×; magnification (I)-10×.

FIG. 2 shows the stages of chronic traumatic encephalopathy (CTE). All images are CP13 immunostained 50 μM tissue sections, and some counterstained with cresyl violet. Original magnification top row—100×, all others—200×.

FIG. 3 are graphs of age of onset in years of CTE in H1/H1 homozygotes (n=24) and total H2 subjects (H1/H2+H2/H2) (n=9) (FIG. 3A), disease duration in years in H1/H1 homozygotes (n=23) and total H2 subjects (n=8) (FIG. 3B), age of onset of disease in years in APOE ε4 genotype (n=13) and non-APOE ε4 genotype (n=19) (FIG. 3C), and disease duration in years in APOE ε4 genotype (n=12) and non-APOE ε4 genotype (n=19) (FIG. 3D). Horizontal bars represent the mean values±standard error of the mean. Comparisons were made using the Student's t-test.

FIG. 4 are graphs of CTE stage in H1/H1 homozygotes (n=25) and total H2 (H1/H2+H2/H2) (n=11) (FIG. 4A); age of death in years of H1/H1 homozygotes (n=25) and total H2 (n=11) (FIG. 4B); stage to age ratio in H1/H1 homozygotes (n=25) and total H2 (n=11) (FIG. 4C); CTE stage in APOE ε4 genotype (n=13) and non-APOE ε4 genotype (n=23) (FIG. 4D); age of death in years of APOE ε4 genotype (n=13) and non-APOE ε4 genotype (n=23) (FIG. 4E); and stage to age ratio in APOE ε4 genotype (n=13) and non-APOE genotype (n=23) (FIG. 4F). Horizontal bars represent the mean values±standard error of the mean. Comparisons were made using the Student's t-test.

### DETAILED DESCRIPTION OF THE INVENTION

### Definitions

The terms used in this specification generally have their ordinary meanings in the art, within the context of this invention and the specific context where each term is used. Certain terms are discussed below, or elsewhere in the specification, to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of the invention or any exemplified term. Likewise, the invention is not limited to its preferred embodiments.

The term “subject” as used in this application means an animal with an immune system such as avians and mammals. Mammals include canines, felines, rodents, bovine, equines, porcines, ovines, and primates. Avians include, but are not limited to, fowls, songbirds, and raptors. Thus, the invention can be used in veterinary medicine, e.g., to treat companion animals, farm animals, laboratory animals in zoological parks, and animals in the wild. The invention is particularly desirable for human medical applications.

The term “patient” as used in this application means a human subject. In some embodiments of the present invention, the “patient” is one suffering with chronic traumatic encephalopathy or CTE.

“Chronic traumatic encephalopathy” and “CTE” will be used interchangeably and is a tauopathy characterized by a constellation of progressive and debilitating neurological symptoms including affective lability, explosivity, irritability, poor attention, executive dysfunction, amnestic symptoms, suicidal ideation, parkinsonism, motor neuron disease, and dementia.

A “tauopathy” is a neurodegenerative disease characterized by the accumulation of tau protein in the brain. These tauopathies include, but are not limited to, Alzheimer's disease, tangle predominant dementia, progressive supranuclear palsy, chronic traumatic encephalopathy, Parkinson's disease, frontotemporal dementia with Parkinsonism linked to chromosome 17 (FTLD-tau), frontotemporal dementia, ganglioglioma, gangliocytoma, meningioangiomatosis, Pick's disease, and corticobasal degeneration.

The terms “screen” and “screening” and the like as used herein means to test a subject or patient to determine if they have a particular illness or disease, in this case CTE. The term also means to test an agent to determine if it has a particular action or efficacy.

The terms “diagnosis”, “diagnose”, diagnosing” and the like as used herein means to determine what physical disease or illness a subject or patient has, in this case CTE.

The terms “identification”, “identify”, “identifying” and the like as used herein means to recognize a disease in a subject or patient, in this case CTE. The term also means to recognize an agent as being effective for a particular use.

The terms “prediction”, “predict”, “predicting” and the like as used herein means to tell in advance based upon special knowledge.

The term “prognosis”, “prognose”, “prognosing” and the like as used herein means to make a prediction on the outcome and course of a disease, in this case CTE.

The term “reference value” as used herein means an amount of a quantity of a particular protein or nucleic acid in a sample from a healthy control.

The term “healthy control” would be a human subject who is not suffering from dementing illness and has normal cognitive and neurologic function. Moreover, it is preferred that the healthy control be age-matched to the subject, within a reasonable range.

The terms “treat”, “treatment”, and the like refer to a means to slow down, relieve, ameliorate or alleviate at least one of the symptoms of the disease, or reverse the disease after its onset.

The terms “prevent”, “prevention”, and the like refer to acting prior to overt disease onset, to prevent the disease from developing or minimize the extent of the disease or slow its course of development.

The term “in need thereof” would be a subject known or suspected of having or being at risk of CTE, such as a subject who is in the military or plays sports or is considering either activity, and/or has already suffered a TBI incident or incidents.

The term “agent” as used herein means a substance that produces or is capable of producing an effect and would include, but is not limited to, chemicals, pharmaceuticals, biologics, small organic molecules, antibodies, nucleic acids, peptides, and proteins.

The phrase “therapeutically effective amount” is used herein to mean an amount sufficient to cause an improvement in a clinically significant condition in the subject, or delays or minimizes or mitigates one or more symptoms associated with the disease, or results in a desired beneficial change of physiology in the subject.

The terms “MAPT”, and “MAPT locus” are used interchangeably in this application and mean the microtubule-associated protein tau gene.

The terms “3′UTR” or “3′ UTR of the MAPT locus” are used interchangeably in this application and mean the critical cis-acting regulatory elements that are capable of regulating gene expression on the post-transcriptional level by influencing mRNA stability and localization, among other functions (Aronov et al. (2001); Aronov et al. (1999)).

A “promoter” or “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase. The promoter may be operatively associated with other expression control sequences, including enhancer and repressor sequences.

The term “antisense DNA” is the non-coding strand complementary to the coding strand in double-stranded DNA.

The term “genomic DNA” as used herein means all DNA from a subject including coding and non-coding DNA, and DNA contained in introns and exons.

As used herein, the term “isolated” and the like means that the referenced material is free of components found in the natural environment in which the material is normally found. In particular, isolated biological material is free of cellular components. In the case of nucleic acid molecules, an isolated nucleic acid includes a PCR product, an isolated mRNA, a cDNA, an isolated genomic DNA, or a restriction fragment. In another embodiment, an isolated nucleic acid is preferably excised from the chromosome in which it may be found. Isolated nucleic acid molecules can be inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated material may be, but need not be, purified.

The term “purified” and the like as used herein refers to material that has been isolated under conditions that reduce or eliminate unrelated materials, i.e., contaminants. For example, a purified protein is preferably substantially free of other proteins or nucleic acids with which it is associated in a cell; a purified nucleic acid molecule is preferably substantially free of proteins or other unrelated nucleic acid molecules with which it can be found within a cell. As used herein, the term “substantially free” is used operationally, in the context of analytical testing of the material. Preferably, purified material substantially free of contaminants is at least 50% pure; more preferably, at least 90% pure, and more preferably still at least 99% pure. Purity can be evaluated by chromatography, gel electrophoresis, immunoassay, composition analysis, biological assay, and other methods known in the art.

The term “nucleic acid hybridization” refers to anti-parallel hydrogen bonding between two single-stranded nucleic acids, in which A pairs with T (or U if an RNA nucleic acid) and C pairs with G. Nucleic acid molecules are “hybridizable” to each other when at least one strand of one nucleic acid molecule can form hydrogen bonds with the complementary bases of another nucleic acid molecule under defined stringency conditions. Stringency of hybridization is determined, e.g., by (i) the temperature at which hybridization and/or washing is performed, and (ii) the ionic strength and (iii) concentration of denaturants such as formamide of the hybridization and washing solutions, as well as other parameters. Hybridization requires that the two strands contain substantially complementary sequences. Depending on the stringency of hybridization, however, some degree of mismatches may be tolerated. Under “low stringency” conditions, a greater percentage of mismatches are tolerable (i.e., will not prevent formation of an anti-parallel hybrid).

The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. Vectors include, but are not limited to, plasmids, phages, and viruses.

Vectors typically comprise the DNA of a transmissible agent, into which foreign DNA is inserted. A common way to insert one segment of DNA into another segment of DNA involves the use of enzymes called restriction enzymes that cleave DNA at specific sites (specific groups of nucleotides) called restriction sites. A “cassette” refers to a DNA coding sequence or segment of DNA that codes for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct” or “gene construct.” A common type of vector is a “plasmid”, which generally is a self-contained molecule of double-stranded DNA, usually of bacterial origin, that can readily accept additional (foreign) DNA and which can readily introduced into a suitable host cell. A plasmid vector often contains coding DNA and promoter DNA and has one or more restriction sites suitable for inserting foreign DNA. Coding DNA is a DNA sequence that encodes a particular amino acid sequence for a particular protein or enzyme. Promoter DNA is a DNA sequence which initiates, regulates, or otherwise mediates or controls the expression of the coding DNA. Promoter DNA and coding DNA may be from the same gene or from different genes, and may be from the same or different organisms. A large number of vectors, including plasmid and fungal vectors, have been described for replication and/or expression in a variety of eukaryotic and prokaryotic hosts. Non-limiting examples include pKK plasmids (Clonetech), pUC plasmids, pET plasmids (Novagen, Inc., Madison, Wis.), pRSET or pREP plasmids (Invitrogen, San Diego, Calif.), or pMAL plasmids (New England Biolabs, Beverly, Mass.), and many appropriate host cells, using methods disclosed or cited herein or otherwise known to those skilled in the relevant art. Recombinant cloning vectors will often include one or more replication systems for cloning or expression, one or more markers for selection in the host, e.g. antibiotic resistance, and one or more expression cassettes.

The term “host cell” means any cell of any organism that is selected, modified, transformed, grown, used or manipulated in any way, for the production of a substance by the cell, for example, the expression by the cell of a gene, a DNA or RNA sequence, a protein or an enzyme. Host cells can further be used for screening or other assays, as described herein.

A “polynucleotide” or “nucleotide sequence” is a series of nucleotide bases (also called “nucleotides”) in a nucleic acid, such as DNA and RNA, and means any chain of two or more nucleotides. A nucleotide sequence typically carries genetic information, including the information used by cellular machinery to make proteins and enzymes. These terms include double or single stranded genomic and cDNA, RNA, any synthetic and genetically manipulated polynucleotide, and both sense and anti-sense polynucleotide. This includes single- and double-stranded molecules, i.e., DNA-DNA, DNA-RNA and RNA-RNA hybrids, as well as “protein nucleic acids” (PNA) formed by conjugating bases to an amino acid backbone. This also includes nucleic acids containing modified bases, for example thio-uracil, thio-guanine and fluoro-uracil.

The nucleic acids herein may be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. The nucleic acids may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, and carbamates) and with charged linkages (e.g., phosphorothioates, and phosphorodithioates). Polynucleotides may contain one or more additional covalently linked moieties, such as, for example, proteins (e.g., nucleases, toxins, antibodies, signal peptides, and poly-L-lysine), intercalators (e.g., acridine, and psoralen), chelators (e.g., metals, radioactive metals, iron, and oxidative metals), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly. Exemplary labels include radioisotopes, fluorescent molecules, biotin, and the like.

The term “polypeptide” as used herein means a compound of two or more amino acids linked by a peptide bond. “Polypeptide” is used herein interchangeably with the term “protein.”

The terms “percent (%) sequence similarity”, “percent (%) sequence identity”, and the like, generally refer to the degree of identity or correspondence between different nucleotide sequences of nucleic acid molecules or amino acid sequences of proteins that may or may not share a common evolutionary origin. Sequence identity can be determined using any of a number of publicly available sequence comparison algorithms, such as BLAST, FASTA, DNA Strider, or GCG (Genetics Computer Group, Program Manual for the GCG Package, Version 7, Madison, Wis.).

The terms “substantially homologous” or “substantially similar” when at least about 80%, and most preferably at least about 90 or 95%, 96%, 97%, 98%, or 99% of the nucleotides match over the defined length of the DNA sequences, as determined by sequence comparison algorithms, such as BLAST, FASTA, and DNA Strider. An example of such a sequence is an allelic or species variant of the specific genes of the invention. Sequences that are substantially homologous can be identified by comparing the sequences using standard software available in sequence data banks, or in a Southern hybridization experiment under, for example, stringent conditions as defined for that particular system.

The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

Genes and Proteins Associated with CTE

CTE is a poorly understood tauopathy. While the effects of repetitive brain damage have been recognized for decades in professional boxers (i.e., dementia pugilistica), surprisingly little is known about the long-term molecular changes that result from repetitive mTBI (Corsellis et al. (1973)). Although the clinical symptoms vary widely, the progression of CTE appears to follow a characteristic course. Blast and physical trauma leads to mechanical stress in the acute setting, shearing cellular structures (e.g., axons, dendrites, synaptic connections, glial processes and blood vessels), resulting in cognitive impairment, or post-concussive syndrome (Stern et al. (2011)). Associated molecular changes with acute trauma include elevation of APP and other proteins (Gentleman et al. (1993)). TBI patients that recover from the initial injury may develop the progressive neurological decline of CTE and neurodegeneration, sometimes many years later (Gavett et al. (2011)). Ultimately, symptoms resurface and the disease progresses until death.

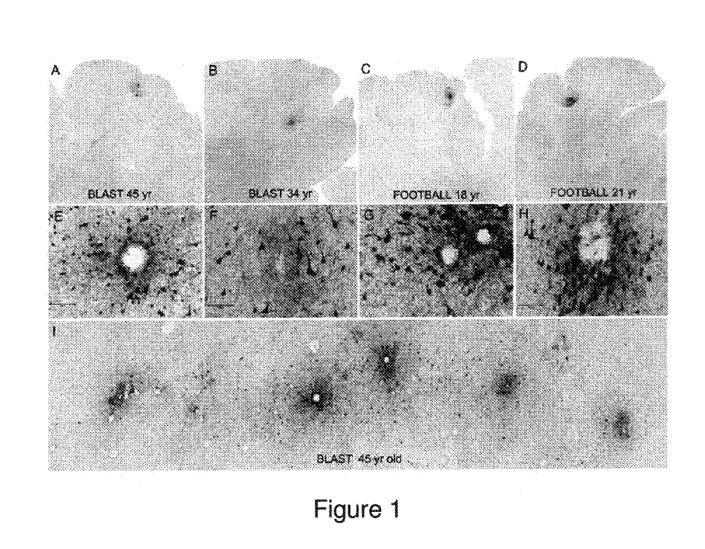

Changes include gross brain atrophy. Microscopically, CTE patients exhibit massive accumulation of abnormal aggregates containing hyperphosphorylated forms of the microtubule-associated protein tau (FIG. 1) (Goldstein et al. (2012); (Gavett et al. (2011)). As many as 85% of CTE patients also exhibit accumulation of 43 kDa TAR DNA binding protein (TDP-43), the primary disease protein in amyotrophic lateral sclerosis, but amyloid plaques that are characteristic of Alzheimer disease and α-synuclein-positive inclusions of Parkinson disease are not prominent (McKee et al. (2009); McKee et al. (2013)).

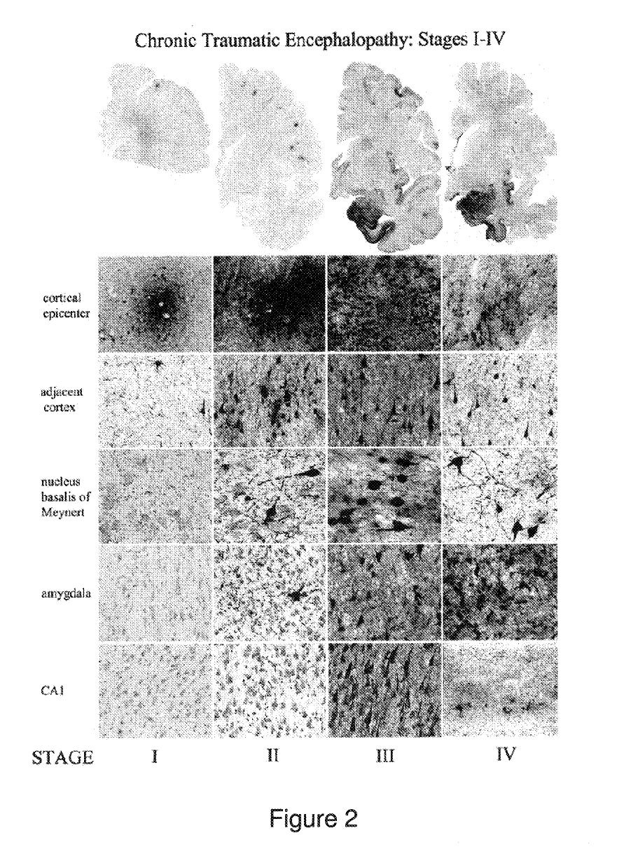

Methods for staging and consensus criteria for the neuropathological diagnosis of CTE have not been widely adopted but were an essential component of the research strategy set forth herein. A staging system has been developed and applied to the subjects with CTE (FIG. 2) (McKee et al. (2013), hereby incorporated by reference in its entirety). Distinctive tauopathic changes were found in the brains of most of these subjects. In stage I, tau pathology is restricted to multiple discrete foci in cerebral (usually frontal) cortex, usually at the depths of sulci around small blood vessels. In stage II, there is localized spread of pathology from these focal epicenters to adjacent cortex. The nucleus basalis of Meynert shows tau neurofibrillary changes; the amygdala and hippocampus (CA1) show the beginnings of tau pathology but are relatively spared. In stage III, tau pathology is widespread, affecting multiple regions of cortex and medial temporal lobe structures, the nucleus basalis, amygdala and CA1 hippocampus are severely affected. In stage IV disease, glial tangles are prominent. There is marked neuronal loss in the cortex, amygdala and hippocampus. Tau NFTs are reduced in size and density.

The studies set forth herein have the potential to elucidate genetic mechanisms of CTE susceptibility.

As is known in the art, there are four alleles of the APOE gene, ε1, ε2, ε3, and ε4. The first is very rare. A significantly elevated frequency of the APOE ε4 allele in CTE subjects compared to controls has been previously reported (Stem et al. (2013)). In this study, this association between the APOE ε4 allele and the risk of developing CTE was confirmed (Example 4), although there does not seem to be any association between APOE ε4 and disease progression or clinical presentation (Examples 6 and 7).

Variation in the tau gene (MAPT) is associated with other tauopathies in the absence of a pathogenic coding region mutation (Wade Martins et al. (2012)). This association stems from the fact that MAPT resides within an approximately 900 kb chromosomal region that has undergone multiple ancestral inversions resulting in two major haplotypes (i.e., H1 and H2) (Stefansson et al. (2005); Zody et al. (2008)). These haplotypes are in complete linkage disequilibrium and do not recombine. Certain tauopathies, including progressive supranuclear palsy, corticobasal degeneration and neurofibrillary tangle-predominant dementia, have been associated with the MAPT H1 haplotype (Baker et al. (1999); DiMaria et al. (2000); Santa-Maria et al. (2012); Janocko et al. (2012)). The association of MAPT with AD remains controversial (Myers et al. (2005); Abraham et al. (2009); Wider et al. (2012)). Surprisingly, Parkinson's disease, not traditionally regarded as a tauopathy, is also associated with H1 (Bekris et al. (2010)). The protective H2 haplotype is most prevalent in Caucasian populations, with an allele frequency of approximately 30%, but is uncommon or rare in other races (Stefansson et al. (2005)).

Until now, the hypothesis that variation in MAPT plays a role in CTE had not been tested.

To ask whether MAPT H1 is associated with CTE, a cohort of neuropathologically-confirmed CTE patients was genotyped, and the frequencies of the H1 and H2 allele compared to the genotype frequencies to a cohort of neuropathologically confirmed non-demented elderly controls. A significant difference was seen between the CTE and control group, with an elevated H1 allele frequency in the CTE patients compared to controls (Example 1).

Next, the MAPT haplotypes were determined in a retrospective analysis of a cohort of athletes with autopsy-confirmed CTE (n=36). First it was determined that there was an association with the H1 allele and CTE (Example 4). When the MAPT haplotypes were correlated to clinical and neuropathological measures of disease severity, MAPT H1 was found to be associated with disease progression and clinical presentation (Examples 6 and 7). Specifically, a significant decrease in disease duration in H1 homozygous CTE patients was observed as compared to those who carry H2 (p=0.019), with an average of 13.4±2.1 years for H1 homozygotes and 25.6±6.0 years for H2 carriers (difference=12.2±4.8 yr, 95% confidence interval=2.1-22.3, R²=0.17). However, there is no difference in the age of symptom onset. Neuropathologically, there was a significant increase in the stage of severity when adjusted for age of death in H1 CTE patients compared to patients that carry H2 (p=0.034).

These findings have invaluable clinical utility. Given the finding that H1 is associated with more severe clinical and neuropathological changes, the MAPT haplotype would be useful as a prognostic marker. While many, if not most, athletes in contact sports, and servicemen suffer from TBI, only some go on to develop CTE. It has now been shown that these persons have an elevated H1 allele frequency that may contribute to their neurodegeneration. Not only does this provide the beginnings of a novel molecular pathway for understanding the pathogenesis of CTE and neurodegeneration in general, this finding can also be used to determine if a person is at higher risk for CTE prior to military deployment or participation in sports.

Additionally, the results herein also have the potential to shed light on the pathogenesis of neurofibrillary degeneration in general. How MAPT haplotypes influence tauopathy is unknown, but H1 is not associated with a known toxic coding region mutation. Furthermore, the CTE patients in this study have neither a family history nor clinical or neuropathological signatures of frontotemporal lobar degeneration (FTLD). Thus, the mechanism of H1 risk most likely stems from genetic variation in regulatory genomic elements that influences tau expression.

In the brain, tau is predominantly present as six major isoforms derived from alternative splicing of MAPT of exons 2, 3 and 10 (Morris et al. (2011)). Inclusion of exon 10 results in a tau protein with four microtubule binding domain repeats (4R), exclusion results in tau with three (3R). Alternative splicing of exons 2 and 3 towards the N-terminus of the gene gives a total of six isoforms Importantly, increased expression of 4R tau mRNA isoforms has been suggested to underlie the risk associated with H1 (Myers et al. (2007)), albeit controversially (Hayesmoore et al. (2009); Trabzuni et al. (2012)). This hypothesis is consistent with the association of progressive supranuclear palsy, a 4R tauopathy, with MAPT H1, suggesting that H1 may cause alterations in exon 10 splicing. Additionally, in vitro evidence suggests that 4R tau may be more prone to formation of toxic aggregates than 3R tau (Adams et al. (2010); Nonaka et al. 2010)). However, at this time there appears to be no mutation in the H1 allele in the coding regions of the exons thought to be expressed in the brain.

Other factors may play a role. The association of H1 with other tauopathies, such as neurofibrillary tangle-predominant dementia, in the absence of alterations in tau splicing suggests that other genetic mechanisms also influence tauopathy. Elements in the tau 3′ UTR regulate mRNA translation, stability and localization leading to speculation that polymorphisms in this region underlie disease risk (Aronov et al. (1999); Aronov et al. (1999); Vandrovcova et al. (2010)). They are also the site for microRNA binding.

The tau promoter region could also play a role.

The data set forth herein for the first time shows biomarkers for the screening, identification, and diagnosis of chronic traumatic encephalopathy as well as biomarkers for the prediction and prognostication of the severity and clinical course of CTE.

Specifically, CTE is associated with an increased frequency of the APOE ε4 allele and the MAPT H1 haplotype. Thus, one or both of these characteristics can be used as biomarkers for the screening, the diagnosing, predicting, and/or identifying of CTE. Additionally, the H1 haplotype can be used to determine the severity of CTE.

These biomarkers can also be used as targets for drug screening and basic research.

Lastly, agents that target the H1 MAPT non-coding regions such as the promoter or the 3′ UTR can be used as preventative and therapeutic agents for CTE. An additional therapy and/or preventative for CTE comprises introducing the protective H2 allele or APOE ε2 and/or ε3 to a subject.

### The Apo E ε4 Allele as a Biomarker for CTE

As stated above and shown in Example 4, CTE is associated with an increased frequency of the Apo E ε4 allele. This association can be used to screen for, predict, diagnose and/or identify CTE.

In order to detect the Apo E ε4 allele associated with CTE, a biological sample from a subject at risk for CTE (i.e., a subject who is known to have had TBI or one who will most likely suffer from TBI due participation in sports at any level or military service, or is considering military service or participation in sports), is obtained and prepared and analyzed for the presence of the Apo E allele ε4. This can be achieved in numerous ways, by a diagnostic laboratory, and/or a health care provider. Specifically the presence of ε4 would indicate a diagnosis or increased risk of CTE.

Any method known in the art can be used to detect the presence or absence of the Apo E allele. Preferred methods that can be utilized in this analysis are sequencing, hybridization with probes including Southern blot analysis and dot blot analysis, polymerase chain reaction (PCR), PCR with melting curve analysis, PCR with mass spectrometry, fluorescent in situ hybridization, DNA microarrays, single-strand conformation analysis, and restriction length polymorphism analysis.

The sequence of the APOE gene is known and can be found at chr19:45409034-45412674. As known in the art there are four alleles, ε1, ε2, ε3, and ε4. The differences in the alleles are at positions chr19:45,411,941 (rs429358) and chr19:45,412,079 (rs7412). The ε1 has a C and T at these positions respectively. The ε2 has a T and T at these positions respectively. The ε3 has a T and C at these positions respectively. The ε4 has a C and C at these positions respectively.

The sequences for the various alleles are set forth herein: ε1 is set forth in SEQ ID NO: 8; ε2 is set forth in SEQ ID NO: 6; ε3 is set forth in SEQ ID NO: 7, and ε4 is set forth in SEQ ID NO: 1.

The present invention includes the use of the DNA or antisense DNA of the nucleotide sequence of the APOE ε4 allele, or SEQ ID NO: 1 as well as the DNA or antisense DNA of other alleles found in SEQ ID NOs: 6-8.

The present invention also includes recombinant constructs comprising the DNA comprising the nucleotide sequence of the APOE ε4 allele, or SEQ ID NO: 1 or the other APOE alleles or SEQ ID NOs: 6-8, or the antisense DNA comprising the nucleotide sequence of the APOE ε4 allele, or SEQ ID NO: 1 or the other APOE alleles or SEQ ID NOs: 6-8, and a vector, that can be expressed in a transformed host cell. The present invention also includes the host cells transformed with the recombinant construct comprising DNA comprising the nucleotide sequence of the APOE ε4 allele, or SEQ ID NO: 1 or the other APOE alleles or SEQ ID NOs: 6-8, or the antisense DNA comprising the nucleotide sequence of the APOE ε4 allele, or SEQ ID NO: 1, or the other APOE alleles or SEQ ID NOs: 6-8 and a vector.

Such DNA sequences, no matter how obtained, are useful in the methods set forth herein for diagnosing or predicting CTE. In the simplest embodiment of the present invention DNA isolated and prepared from a sample of biological tissue and/or bodily fluid from a subject with a known risk of CTE is compared to the known sequences of the Apo E ε4 allele to screen for, predict, or confirm a diagnosis of CTE.

The isolated DNA can also be used as the basis for probes and primers for use in additional diagnostic procedures for CTE.

### The H1 Haplotype as a Biomarker for CTE

As stated above and shown in the Examples, CTE is closely associated with the H1 haplotype of the MAPT locus. With this in mind, one embodiment of the present invention is a test in an individual for the presence of one or the other alleles, MAPT H1 or H2. Such individuals would include, but is not limited to, those who are in the military and those of all ages who play or are considering playing a sport. The results of the individual would be compared to the known frequencies of the alleles. Specifically, as stated above and shown by the data, the H2 allele is protective as to the development of neurodegeneration. The H2 allele is more frequently associated with control subjects who have experienced normal aging, whereas the H1 allele is more frequently associated with those who have pathologies, specifically CTE. Additionally, the H1 haplotype has been shown to be associated with a more rapid decline from the disease. Thus, a result showing the individual possessed the H1 allele would be an indication that the individual is at greater risk for developing CTE and other types of neurodegeneration from the trauma that can be part of military duty and sports. In opposite, if the individual possesses the H2 allele, they would be at lower risk for the development of CTE and neurodegeneration in general. Individuals (and their parents if minors) can then make educated decisions regarding military service and participation in sports. These individuals would also know that they should take extra precaution to avoid TBI, and after an incidence of TBI, such as taking extra time to recover from concussions.

Additionally, since at the current time the only definitive diagnosis of CTE is from brain tissue after death, the H1 haplotype can be used to identify and diagnose CTE in a subject who has already suffered TBI.

Thus, an embodiment of the present invention is the use of this association to screen for, predict, diagnose, prognose, and/or identify CTE.

In order to detect the H1 haplotype associated with CTE, a biological sample from a subject at risk for CTE is obtained and prepared and analyzed for the presence of the H1 haplotype. This can be achieved in numerous ways, by a diagnostic laboratory, and/or a health care provider.

Any method known in the art can be used to detect the presence or absence of the H1 haplotype. Preferred methods that can be utilized in this analysis are sequencing, hybridization with probes including Southern blot analysis and dot blot analysis, polymerase chain reaction (PCR), PCR with melting curve analysis, PCR with mass spectrometry, fluorescent in situ hybridization, DNA microarrays, single-strand conformation analysis, and restriction length polymorphism analysis.

The approximate 2 Mb H1 haplotype is found on chromosome 17q21 between base pairs 43,000,000 and 45,000,000, and is obtainable in the genome browser at chr17:43,000,000-45,000,000 (UCSC Genome Browser on Human February 2009 (GRCh37/hg19) Assembly). The H1 haplotype of the MAPT 3′ UTR is found at chromosome 17 between base pairs 44,101295 and 44,105,727 and is set forth in SEQ ID NO: 2. The MAPT H1 promoter is located at chr17:43,951,748-43,971,747, and is set forth in SEQ ID NO: 3.

One embodiment of the present invention is the use of the isolated DNA encoding the H1 haplotype of the MAPT gene, found on chromosome 17 between base pairs 43,000,000 and 45,000,000, and obtainable in the genome browser at chr17:43,000,000-45,000,000 (UCSC Genome Browser on Human February 2009 (GRCh37/hg19) Assembly), as a diagnostic and/or prognostic for CTE.

Further embodiments of the present invention are methods of using the isolated DNA of the H1 haplotype of the 3′ UTR of the MAPT gene comprising the nucleotide sequence of SEQ ID NO: 2.

Further embodiments of the present invention are methods of using the isolated DNA of the H1 haplotype of the promoter of the MAPT gene comprising the nucleotide sequence of SEQ ID NO: 3.

The present invention also includes the use of the antisense DNA of the H1 haplotype, as well as the DNA sequence listed in SEQ ID NOs: 2 and 3.

The present invention also includes recombinant constructs comprising the DNA comprising the nucleotide sequence of H1 haplotype of the MAPT locus, or SEQ ID NOs: 2 or 3, or the antisense DNA comprising the nucleotide sequence of H1 haplotype of the MAPT gene or SEQ ID NOs: 2 or 3, and a vector, that can be expressed in a transformed host cell. The present invention also includes the host cells transformed with the recombinant construct comprising DNA comprising the nucleotide sequence of H1 haplotype of the MAPT locus, or SEQ ID NOs: 2 or 3, or the antisense DNA comprising the nucleotide sequence of H1 haplotype of the MAPT locus, or SEQ ID NOs: 2 or 3, and a vector.

Such DNA sequences, no matter how obtained, are useful in the methods set forth herein for diagnosing CTE. In the simplest embodiment of the present invention, DNA isolated and prepared from a sample of biological tissue and/or bodily fluid from a subject at risk for CTE is compared to the DNA sequence of the H1 haplotype of the MAPT locus and/or SEQ ID NO: 2 and/or SEQ ID NO: 3 to predict, identify and/or diagnosis CTE or an increased risk of CTE.

The isolated DNA can also be used as the basis for probes and primers for used in additional diagnostic procedures for CTE.

### Screening and Diagnostic Methods and Assays Utilizing the APOE ε4 Allele and the MAPT H1 Haplotype

Several methods can be used to screen for, diagnose, predict, and identify CTE in a subject utilizing the surprising discovery of the association of the APOE ε4 allele, and the MAPT H1 haplotype to individuals with CTE.

The most direct method for screening for and diagnosing CTE is to obtain a sample of biological tissue or bodily fluid from the subject and extracting, isolating and/or purifying the nucleic acid (e.g., genomic DNA, cDNA, RNA) from the tissue or fluid.

The nucleic acid can be obtained from any biological tissue. Preferred biological tissues include, but are not limited to, brain and epidermis.

The nucleic acid can be obtained from any bodily fluid. Preferred bodily fluids include, but are not limited to, cerebrospinal fluid, whole blood, buffy coat, serum, plasma, saliva, sweat, and urine.

The nucleic acid is extracted, isolated and purified from the cells of the tissue or fluid by methods known in the art. The nucleic acid, e.g., DNA is then sequenced.

In one embodiment, the nucleic acid is sequenced at the APOE locus, and the sequenced nucleic acid is then inspected at the APOE locus for the APOE ε4 allele. Specifically, the DNA from the patient is compared to the DNA of one or all of the nucleotides comprising the sequences of SEQ ID NOs: 1, 6, 7 and/or 8. The presence of the ε4 allele set forth in SEQ ID NO: 1 would indicate the patient has CTE or a risk of CTE, and the absence of the ε4 allele set forth in SEQ ID NO: 1 and/or the presence of the ε2 allele set forth in SEQ ID NO: 6 and/or the ε3 allele set forth in SEQ ID NO: 7 and/or the presence of the ε1 allele set forth in SEQ ID NO: 8, would indicate the patient does not have CTE or is at a low risk for CTE.

In another embodiment, the nucleic acid is sequenced at the MAPT locus and the sequenced nucleic acid is inspected at the MAPT locus for either the H1 haplotype. Specifically, the isolated, purified and sequenced DNA from the patient is compared to the DNA with the nucleotide sequences of one or all of the H1 haplotype found on chromosome 17 between base pairs 43,000,000 and 45,000,000, and obtainable in the genome browser at chr17:43,000,000-45,000,000 (UCSC Genome Browser on Human February 2009 (GRCh37/hg19) Assembly), and/or SEQ ID NOs: 2 and/or 3. The comparison can be made to one sequence, or all sequences. The presence of any of these DNA sequences in the DNA from the biological tissue or fluid of the subject would indicate the subject has CTE or is at a higher risk for developing CTE or will have a more rapid clinical decline from CTE.

Alternatively, or additionally, the comparison of the DNA form the patient is compared with the H2 haplotype and/or SEQ ID NO: 4 and/or 5. The presence of the H2 haplotype would indicate the subject does not have CTE or is at a lower risk for developing CTE. The H2 haplotype of the MAPT is found at chromosome 17q21, and the H2 3′UTR is found at chromosome 17 between base pairs 76,2196-76,6698 and is set forth in SEQ ID NO: 4. The H2 promoter is set forth in SEQ ID NO: 5 (Stefansson et al. (2005)).

A preferred embodiment includes a comparison of the nucleotide sequence from the subject to APOE ε4 allele sequence, the MAPT H1 sequence, the MAPT H1 promoter sequence, and the MAPT H1 UTR sequence.

The DNA from the subject can be sequenced by direct DNA sequencing either manual or automated by methods known in the art such as Sanger sequencing, dideoxy sequencing, and automated fluorescent sequencing.

Screening and diagnostic method of the current invention may involve the amplification of the APOE locus, MAPT locus, or the 3′UTR of the MAPT locus. A preferred method for target amplification of nucleic acid sequences is using polymerases, in particular polymerase chain reaction (PCR). PCR or other polymerase-driven amplification methods obtain millions of copies of the relevant nucleic acid sequences which then can be used as substrates for probes or sequenced or used in other assays.

Amplification using polymerase chain reaction is particularly useful in the embodiments of the current invention. PCR is a rapid and versatile in vitro method for amplifying defined target DNA sequences present within a source of DNA. Usually, the method is designed to permit selective amplification of a specific target DNA sequence(s) within a heterogeneous collection of DNA sequences (e.g. total genomic DNA or a complex cDNA population). To permit such selective amplification, some prior DNA sequence information from the target sequences is required. This information is used to design two oligonucleotide primers (amplimers) which are specific for the target sequence and which are often about 15-25 nucleotides long.

Of particular usefulness in the current invention is the use of oligonucleotide primers to discriminate between target DNA sequences that differ by a single nucleotide in the region of interest called allele-specific PCR. These allele-specific primers will anneal only to the alleles of interest. In this case, the primers of the current invention made from the nucleotide sequence of the APOE ε4 allele, the MAPT H1 haplotype, and/or nucleotide sequence set forth in SEQ ID NOs: 1 or 2 or 3 can be used as a screen of the genomic DNA from the subject. Only if the DNA contains the ε4 allele and/or H1 haplotype of the MAPT locus will the primers anneal and amplify the product. Alternatively or additionally, primers can be made from the APOE ε1, ε2, ε3 and/or H2 haplotype and/or SEQ ID NOs: 4-8 and used to screen the DNA from the subject.

Mutation detection using the 5′→3′ exonuclease activity of Taq DNA polymerase (TaqMan™ assay) can also be used as a screening and diagnostic method of the current invention. Such an assay involves hybridization of three primers, the third primer being intended to bind just downstream of one of the conventional primers which should be allele-specific. The additional primer carries a blocking group at the 3′ terminal nucleotide so that it cannot prime new DNA synthesis and at its 5′ end carries a labeled group. In modern versions of the assay, the label is a fluorogenic group and the third primer also carries a quencher group. If the upstream primer which is bound to the same strand is able to prime successfully, Taq DNA polymerase will extend a new DNA strand until it encounters the third primer in which case its 5′→3′ exonuclease will degrade the primer causing release of separate nucleotides containing the dye and the quencher, and an observable increase in fluorescence.

PCR with melting curve analysis can also be used with the disclosed biomarkers to screen for, identify and diagnose CTE. PCR with melting curve analysis is an extension of PCR where the fluorescence is monitored over time as the temperature changes. Duplexes melt as the temperature increases and the hybridization of both PCR products and probes can be monitored. The temperature-dependent dissociation between two DNA-strands can be measured using a DNA-intercalating fluorophore, such as SYBR green, EvaGreen or fluorophore-labelled DNA probes. In the case of SYBR green (which fluoresces 1000-fold more intensely while intercalated in the minor groove of two strands of DNA), the dissociation of the DNA during heating is measurable by the large reduction in fluorescence that results. Alternatively, juxtapositioned probes (one featuring a fluorophore and the other, a suitable quencher) can be used to determine the complementarity of the probe to the target sequence. This technique is sensitive enough to detect single-nucleotide polymorphisms (SNP) and can distinguish between various alleles by virtue of the dissociation patterns produced.

PCR with mass spectrometry uses mass spectrometry to detect the end product.

Primer pairs are used and tagged with molecules of known masses, known as MassCodes. If DNA from any of the agent of primer panel is present, it will be amplified. Each amplified product will carry its specific Masscodes. The PCR product is then purified to remove unbound primers, dNTPs, enzyme and other impurities. Finally, the purified PCR products are subject of ultraviolet as the chemical bond with nucleic acid and primers are photolabile. As the Masscodes are liberated from PCR products they are detected with a mass spectrometer.

When a probe is to be used to detect the presence of the H1 or H2 haplotype or APOE alleles, the biological sample that is to be analyzed must be treated to extract the nucleic acids. The nucleic acids to be targeted usually need to be at least partially single-stranded in order to form a hybrid with the probe sequence. It the nucleic acid is single stranded, no denaturation is required. However, if the nucleic acid to be probed is double stranded, denaturation must be performed by any method known in the art.

The nucleic acid to be analyzed and the probe are incubated under conditions which promote stable hybrid formation of the target sequence in the probe and the target sequence in the nucleic acid. The desired stringency of the hybridization will depend on factors such as the uniqueness of the probe in the part of the genome being targeted, and can be altered by washing procedure, temperature, probe length and other conditions known in the art, as set forth in Maniatis et al. (1982) and Sambrook et al. (1989).

Labeled probes are used to detect the hybrid, or alternatively, the probe is bound to a ligand which labeled either directly or indirectly. Suitable labels and methods for labeling are known in the art, and include biotin, fluorescence, chemiluminescence, enzymes, and radioactivity.

Assays using such probes include Southern blot analysis. In such an assay, a patient sample is obtained, the DNA processed, denatured, separated on an agarose gel, and transferred to a membrane for hybridization with a probe. Following procedures known in the art (e.g., Sambrook et al. (1989)), the blots are hybridized with a labeled probe and a positive band indicates the presence of the target sequence. Southern blot hybridization can also be used to screen for the polymorphisms. In this method, the target DNA is digested with one or more restriction endonucleases, size-fractionated by agarose gel electrophoresis, denatured and transferred to a nitrocellulose or nylon membrane for hybridization. Following electrophoresis, the test DNA fragments are denatured in strong alkali. As agarose gels are fragile, and the DNA in them can diffuse within the gel, it is usual to transfer the denatured DNA fragments by blotting on to a durable nitrocellulose or nylon membrane, to which single-stranded DNA binds readily. The individual DNA fragments become immobilized on the membrane at positions which are a faithful record of the size separation achieved by agarose gel electrophoresis. Subsequently, the immobilized single-stranded target DNA sequences are allowed to associate with labeled single-stranded probe DNA. The probe will bind only to related DNA sequences in the target DNA, and their position on the membrane can be related back to the original gel in order to estimate their size.

Dot-blot hybridization can also be used to screen for the ε4 allele and/or the H1 haplotype. Nucleic acid including genomic DNA, cDNA and RNA is obtained from the subject, denatured and spotted onto a nitrocellulose or nylon membrane and allowed to dry. The membrane is exposed to a solution of labeled single stranded probe sequences and after allowing sufficient time for probe-target heteroduplexes to form, the probe solution is removed and the membrane washed, dried and exposed to an autoradiographic film. A positive spot is an indication of the target sequence in the DNA of the subject and a no spot an indication of the lack of the target sequence in the DNA of the subject.

A particularly useful application of dot blotting is the use of allele-specific oligonucleotide (ASO) probes. This method distinguishes between alleles that differ by even a single nucleotide substitution. ASO probes are using between 15-20 nucleotides long and are employed under hybridization conditions at which the DNA duplex between the probe and the target are stable only if there is a perfect base complementarity between them.

A further embodiment is the use of ASO reverse dot blotting, wherein an oligonucleotide probe is fixed on a filter or membrane and the target DNA is labeled and provided in a solution. Positive binding of labeled target DNA to a specific oligonucleotide on the membrane is taken to mean that the target DNA has the specific sequence.

DNA microarrays can also be used to screen for the APOE alleles and/or MAPT haplotype. The surfaces involved are glass rather than porous membranes and similar to reverse dot-blotting, the DNA microarray technologies employ a reverse nucleic acid hybridization approach: the probes consist of unlabeled DNA fixed to a solid support (the arrays of DNA or oligonucleotides) and the target is labeled and in solution.

DNA microarray technology also permits an alternative approach to DNA sequencing by permitting by hybridization of the target DNA to a series of oligonucleotides of known sequence, usually about 7-8 nucleotides long. If the hybridization conditions are specific, it is possible to check which oligonucleotides are positive by hybridization, feed the results into a computer and use a program to look for sequence overlaps in order to establish the required DNA sequence. DNA microarrays have permitted sequencing by hybridization to oligonucleotides on a large scale.

Single strand conformation analysis can also be used to determine if the purified and isolated DNA from a subject has particular allele, haplotype or SNP. The conformation of the single-stranded DNA can alter based upon a single base change in the sequence, causing the DNA to migrate differently on electrophoresis. The analysis can involve four steps: (1) polymerase chain reaction (PCR) amplification of DNA sequence of interest; (2) denaturation of double-stranded PCR products; (3) cooling of the denatured DNA (single-stranded) to maximize self-annealing; and (4) detection of mobility difference of the single-stranded DNAs by electrophoresis under non-denaturing conditions. Additionally, the SSCP mobility shifts must be visualized which is done by the incorporation of radioisotope labeling, silver staining, fluorescent dye-labeled PCR primers, and more recently, capillary-based electrophoresis.

### Probes and Primers

Further embodiments of the present invention include probes comprising some or all of the DNA comprising the nucleotide sequence of SEQ ID NOs: 1, and 6-8 and probes comprising some or all of the DNA with the antisense nucleotide sequence of SEQ ID NOs: 1 and 6-8. These probes can be used to detect the Apo E ε4 allele associated with CTE in a sample of DNA from a subject and/or the ε1, ε2 and/or ε3 alleles not associated with CTE.

Further embodiments of the present invention include probes comprising some or all of the DNA comprising the nucleotide sequence of the H1 haplotype of the MAPT locus, and SEQ ID NOs: 2 and 3, and the H2 haplotype of the MAPT locus and SEQ ID NOs: 4 and 5, and probes comprising some or all of the DNA comprising the antisense nucleotide sequence of H1 haplotype of the MAPT locus, and SEQ ID NOs: 2 and 3, and the H2 haplotype of the MAPT locus and SEQ ID NO: 4 and 5. These probes can be used to detect H1 haplotype associated with CTE, or the protective H2 haplotype, in a sample of DNA from a subject.

Probes contemplated for use in the screening and diagnostic assays of the present invention can be made by any method known in the art, including the procedures outlined below.

In standard nucleic acid hybridization assays, probe must be is labeled in some way, and must be single stranded. Oligonucleotide probes are short (typically 15-50 nucleotides) single-stranded pieces of DNA made by chemical synthesis: mononucleotides are added, one at a time, to a starting mononucleotide, conventionally the 3′ end nucleotide, which is bound to a solid support. Generally, oligonucleotide probes are designed with a specific sequence chosen in response to prior information about the target DNA. Oligonucleotide probes are often labeled by incorporating a ³²P atom or other labeled group at the 5′ end.

Conventional DNA probes are isolated by cell-based DNA cloning or by PCR. In the former case, the starting DNA may range in size from 0.1 kb to hundreds of kilobases in length and is usually (but not always) originally double-stranded. PCR-derived DNA probes have often been less than 10 kb long and are usually, but not always, originally double-stranded.

DNA probes are usually labeled by incorporating labeled dNTPs during an in vitro DNA synthesis reaction by many different methods including nick-translation, random primed labeling, PCR labeling or end-labeling.

Labels can be radioisotopes such as ³²P, ³³P, ³⁵S and ³H, which can be detected specifically in solution or, more commonly, within a solid specimen, such as autoradiography. ³²P has been used widely in Southern blot hybridization, and dot-blot hybridization.

Nonisotopic labeling systems which use nonradioactive probes can also be used in the current invention. Two types of non-radioactive labeling include direct nonisotopic labeling, such as one involving the incorporation of modified nucleotides containing a fluorophore. The other type is indirect nonisotopic labeling, usually featuring the chemical coupling of a modified reporter molecule to a nucleotide precursor. After incorporation into DNA, the reporter groups can be specifically bound by an affinity molecule, a protein or other ligand which has a very high affinity for the reporter group. Conjugated to the latter is a marker molecule or group which can be detected in a suitable assay. This type of labeling would include biotin-streptavidin and digoxigenin.

Primers for use in the various assays of the present invention are also an embodiment of the present invention. Primers useful for the methods of screening and diagnosis of the present invention are also contemplated by the invention and can be prepared by method known in the art as outlined below, using the sequences of the MAPT 3′UTR, and H1 and H2 haplotype of the MAPT gene, as well as the sequences of the APOE ε4, ε1, ε2, and ε3 alleles.

The specificity of amplification depends on the extent to which the primers can recognize and bind to sequences other than the intended target DNA sequences. For complex DNA sources, such as total genomic DNA from a mammalian cell, it is often sufficient to design two primers about 20 nucleotides long. This is because the chance of an accidental perfect match elsewhere in the genome for either one of the primers is extremely low, and for both sequences to occur by chance in close proximity in the specified direction is normally exceedingly low. Although conditions are usually chosen to ensure that only strongly matched primer-target duplexes are stable, spurious amplification products can nevertheless be observed. This can happen if one or both chosen primer sequences contain part of a repetitive DNA sequence, and primers are usually designed to avoid matching to known repetitive DNA sequences, including large runs of a single nucleotide

After the primers are added to denatured template DNA, they bind specifically to complementary DNA sequences at the target site. In the presence of a suitably heat-stable DNA polymerase and DNA precursors (the four deoxynucleoside triphosphates, dATP, dCTP, dGTP and dTTP), they initiate the synthesis of new DNA strands which are complementary to the individual DNA strands of the target DNA segment, and which will overlap each other.

### Use of Levels of Apo E ε4 Polypeptide as a Screening and Diagnosis Method for CTE

As stated above, and shown in Example 4, CTE is associated with higher levels of the Apo E ε4. Thus, one embodiment of the present invention is the screening, diagnosis, prediction or identification of CTE in a subject, by detection of increased levels or quantities of the Apo E ε4 polypeptide in a sample from a subject at risk for CTE including, but not limited to, those who have suffered TBI or those who are in the military and those of all ages who play a sport, or are contemplating these activities. Alternatively or additionally, an embodiment of the present invention is the screening, diagnosis, prediction or identification of CTE in a subject, by detection of decreased levels or quantities of the Apo E ε1 or ε2 or ε3 polypeptides in a sample from a subject at risk for CTE including, but not limited to, those who have suffered TBI or those who are in the military and those of all ages who play a sport, or are contemplating these activities.

A sample of biological tissue or bodily fluid from a subject is obtained.

The protein sample can be obtained from any biological tissue. Preferred biological tissues include, but are not limited to, brain, epidermal, whole blood, and plasma.

The protein sample can be obtained from any bodily fluid. Preferred bodily fluids include, but are not limited to, cerebrospinal fluid, plasma, saliva, sweat, and urine.

Protein is purified and/or isolated from the sample using any method known in the art including but not limited to immunoaffinity chromatography.

Any method known in the art can be used, but preferred methods for detecting increased levels or quantities of Apo E in a protein sample include quantitative Western blot, immunoblot, quantitative mass spectrometry, enzyme-linked immunosorbent assays (ELISAs), radioimmunoassays (RIA), immunoradiometric assays (IRMA), and immunoenzymatic assays (IEMA) and sandwich assays using monoclonal and polyclonal antibodies.

Antibodies are a preferred method of detecting Apo E polypeptides in a sample. Such antibodies are available commercially or can be made by conventional methods known in the art. Such antibodies can be monoclonal or polyclonal and fragments thereof, and immunologic binding equivalents thereof. The term “antibody” means both a homologous molecular entity as well as a mixture, such as a serum product made up of several homologous molecular entities.

In a preferred embodiment, such antibodies will immunoprecipitate Apo E polypeptides from a solution as well as react with Apo E polypeptides on a Western blot, or immunoblot, ELISA, and other assays listed above. In another preferred embodiment, these antibodies will react and detect Apo E ε4, ε1, β2, or ε3 polypeptide in frozen tissue section, say from a brain biopsy.

Antibodies for use in these assays can be labeled covalently or non-covalently with an agent that provides a detectable signal. Any label and conjugation method known in the art can be used. Labels, include but are not limited to, enzymes, fluorescent agents, radiolabels, substrates, inhibitors, cofactors, magnetic particles, and chemiluminescent agents.

The levels or quantities of Apo E ε4 polypeptide found in a sample are compared to the levels or quantities of these peptides in healthy controls and a deviation in the level or quantity of peptides is looked for. This comparison can be done in many ways. The same assay can be performed simultaneously or consecutively, on a purified and/or isolated protein sample from a healthy control and the results compared qualitatively, e.g., visually, i.e., does the protein sample from the healthy control produce the same intensity of signal as the protein sample from the subject in the same assay, or the results can be compared quantitatively, e.g., a value of the signal for the protein sample from the subject is obtained and compared to a known reference value of the protein in a healthy control.

A higher level or quantity of Apo E polypeptides in a sample from a subject as compared to the reference value of the level or quantity of the peptides in a healthy control would indicate the subject has CTE or a higher risk of CTE.

A lower level or quantity of Apo E ε1, ε2 and/or 3 polypeptide in a sample from a subject as compared to the reference value of the level or quantity of the peptides in a healthy control would indicate the subject has CTE or a higher risk of CTE.

### Kits

Diagnostic and screening assays based upon nucleotide testing can also be incorporated into kits. For example, probes and/or primers for each of the APOE ε4, ε1, ε2, and ε3 alleles, reagents for isolating and purifying nucleic acids from biological tissue or bodily fluid, reagents for performing assays on the isolated and purified nucleic acid, instructions for use, and comparison sequences could be included in a kit for detection of the APOE ε4 allele. Kits for screening and diagnosis utilizing the H1 haplotype of the MAPT locus are also contemplated by the invention. These kits could include probes and/or primers specific for the H1 haplotype, reagents for isolating and purifying nucleic acids from biological tissue or bodily fluid, reagents for performing assays on the isolated and purified nucleic acid, instructions for use, and comparison sequences could be included in a kit for detection of the H1 haplotype.

Kits for screening and diagnosis utilizing the H2 haplotype of the MAPT locus are also contemplated by the invention. These kits could include probes and/or primers specific for the H2 haplotype, reagents for isolating and purifying nucleic acids from biological tissue or bodily fluid, reagents for performing assays on the isolated and purified nucleic acid, instructions for use, and comparison sequences could be included in a kit for detection of the H2 haplotype.

A preferred embodiment is a kit including components for testing for both the MAPT haplotypes and APOE alleles.

Another kit would test for the Apo E ε4, ε1, ε2, and/or ε3 polypeptides and could include antibodies that recognize the peptide of interest, reagents for isolating and/or purifying protein from a biological tissue or bodily fluid, reagents for performing assays on the isolated and purified protein, instructions for use, and reference values or the means for obtaining reference values for the quantity or level of peptides in a control sample.

It is contemplated that all of the diagnostic and screening assays disclosed herein can be in kit form for use by a health care provider and/or a diagnostic laboratory.

### Drug Screening Assays and Research Tools

All of the biomarkers disclosed herein can be used as the basis for drug screening assays and research tools.

In one embodiment, the DNA or RNA comprising the MAPT H1 haplotype or the 3′UTR of the H1 haplotype or the H1 promoter or the APOE ε4 allele or SEQ ID NOs: 1, 2 or 3 is contacted with an agent, and a complex between the DNA or RNA and the agent is detected by methods known in the art. One such method is labeling the DNA or RNA and then separating the free DNA or RNA from that bound to the agent. If the agent binds to the DNA or RNA, the agent would be considered a potential therapeutic for CTE.

In a further embodiment, a nucleotide comprising the MAPT H1 haplotype or the H1 promoter or the 3′UTR of the H1 haplotype or the APOE ε4 allele or SEQ ID NOs: 1, 2 or 3 can be incubated and/or contacted with a potential therapeutic agent. The resulting expression of the nucleotide can be detected and compared to the expression before contact with the agent.

A further embodiment of the present invention is a gene construct comprising the MAPT H1 haplotype or the H1 promoter or the 3′UTR of the H1 haplotype or the APOE ε4 allele or SEQ ID NOs: 1, 2 or 3, and a vector. Sequences can be amplified prior to cloning. These gene constructs can be used for testing of therapeutic agents as well as basic research regarding CTE. These gene constructs can also be used to transform host cells can be transformed by methods known in the art.

The resulting transformed cells can be used for testing for therapeutic agents. Specifically, the host cells can be incubated and/or contacted with a potential therapeutic agent. The resulting expression of the gene construct can be detected and compared to the expression of the gene construct in the cell before contact with the agent.

The expression of the transcripts in host cells can be detected and measured by any method known in the art. The H1 3′UTR or H1 promoter or other DNA can also be linked to other genes with measurable phenotypes. Expression of the gene linked to the H1 3′UTR or other DNA of the H1 haplotype or the APOE ε4 allele or SEQ ID NOs: 1 or 2 or 3, can be measured before and after the contact with a potential therapeutic agent, as well as a naturally occurring peptide or molecule. Such constructs include but are not limited to a dual luciferase reporter gene psiCHECK-2 vector, and tau.

These gene constructs as well as the host cells transformed with these gene constructs can also be the basis for transgenic animals for testing both as research tools and for therapeutic agents. Such animals would include but are not limited to, nude mice and drosophila. Phenotypes can be correlated to the genes and looked at in order to determine the genes effect on the animals as well as the change in phenotype after administration or contact with a potential therapeutic agent.

Additionally, the Apo E ε4 polypeptide can be used in drug screening assays, free in solution, or affixed to a solid support. All of these forms can be used in binding assays to determine if agents being tested form complexes with the peptides, proteins or fragments, or if the agent being tested interferes with the formation of a complex between the peptide or protein and a known ligand.

Thus, the present invention provides for methods and assays for screening agents for treatment of CTE, comprising contacting or incubating the test agent with a Apo E ε4 polypeptide, and detecting the presence of a complex between the polypeptide and the agent or the presence of a complex between the polypeptide and a ligand, by methods known in the art. In such competitive binding assays, the polypeptide or fragment is typically labeled. Free polypeptide is separated form that in the complex, and the amount of free or uncomplexed polypeptide is measured. This measurement indicates the amount of binding of the test agent to the polypeptide or its interference with the binding of the polypeptide to a ligand.

High throughput screening can also be used to screen for therapeutic agents. Small peptides or molecules can be synthesized and bound to a surface and contacted with the polypeptides, and washed. The bound peptide is visualized and detected by methods known in the art.

Antibodies to the polypeptides can also be used in competitive drug screening assays. The antibodies compete with the agent being tested for binding to the polypeptides. The antibodies can be used to find agents that have antigenic determinants on the polypeptides, which in turn can be used to develop monoclonal antibodies that target the active sites of the polypeptides.

The invention also provides for polypeptides to be used for rational drug design where structural analogs of biologically active polypeptides can be designed. Such analogs would interfere with the polypeptide in vivo, such as by non-productive binding to target. In this approach the three-dimensional structure of the protein is determined by any method known in the art including but not limited to x-ray crystallography, and computer modeling. Information can also be obtained using the structure of homologous proteins or target-specific antibodies.

Using these techniques, agents can be designed which act as inhibitors or antagonists of the polypeptides, or act as decoys, binding to target molecules non-productively and blocking binding of the active polypeptide.

Any agents identified in these assays as being effective as a preventative and/or therapeutic for CTE are also embodiments of the invention.

### Treatment and Prevention for CTE

As shown herein, the H1 haplotype of the tau MAPT gene is associated with CTE or an increased risk of CTE. It is also associated with an increase in severity and a hastening of disease progression. Prevention and treatment of CTE can stem from this association. Thus, one embodiment of the present invention is the treatment and/or prevention of CTE by administering an agent that binds to the H1 promoter or 3′UTR or other section of the tau DNA or mRNA derived from the tau MAPT gene to a subject in need thereof. One example of this is a microRNA. A further embodiment is the administration of an agent that increases binding of a naturally occurring molecule, such as a microRNA, to the H1 promoter or 3′UTR or other section of the tau DNA or mRNA in a subject in need thereof, either by increasing the amount or production of the molecule or by increasing binding affinity and/or stability.

A subject in need thereof is defined as a subject known or suspected of having or being at risk of CTE, such as a subject who is in the military or plays a sports and/or has already suffered a TBI incident.

Agents that bind to the H1 promoter would include but are not limited to SP1 and AP-2 transcription factors.

Agents that bind to the H1 3′ UTR include but are not limited to, miRNA, RNA-binding proteins such as embryonic lethal, abnormal vision (ELAV)-like 4 (ELAVL4, or HuD), insulin-like growth factor 2, mRNA-binding protein 1, IGF2BP1 or IMP1/ZBP1, TDP43, and FUS.

As also shown the H2 haplotype of the MAPT locus is protective. Thus, a further method of treatment or prevention of CTE would be supplying the H2 haplotype to a subject in need thereof. The H2 haplotype of the MAPT is found at chromosome 17q21, and the H2 3′UTR is found at chromosome 17 between base pairs 76,2196-76,6698 and is set forth in SEQ ID NO: 4. The H2 promoter is set forth in SEQ ID NO: 5 (Stefansson et al. (2005))

A subject in need thereof is defined as a subject known or suspected of having or being at risk of CTE, such as a subject who is in the military or plays a sports and/or has already suffered a TBI incident.

Classical gene therapies normally require efficient transfer of cloned genes into disease cells so that the introduced genes are expressed at suitably high levels. Following gene transfer, the inserted genes may integrate into the chromosomes of the cell, or remain as extrachromosomal genetic elements (episomes).

For the former situation, the DNA recombines with the endogenous gene that produces the DNA present in the cell. Such recombination requires a double recombination event which results in the conversion of the MAPT H1 allele to the H2 allele.

Vectors for introduction of the DNA in either recombination or extrachromosomal reproduction are known in the art and are discussed herein. Methods for introduction of genes into cells are known in the art and are discussed herein and include electroporation, calcium phosphate co-precipitation, and viral transduction.

One such method for delivering the DNA is receptor mediated endocytosis where the DNA is coupled to a targeting molecule that can bind to a specific cell surface receptor, inducing endocytosis and transfer of the DNA into cells. Coupling is normally achieved by covalently linking poly-lysine to the receptor molecule and then arranging for (reversible) binding of the negatively charged DNA to the positively charged poly-lysine component. Another approach utilizes the transferrin receptor or folate receptor which is expressed in many cell types. When producing the DNA for this method of administration, the DNA could be manufactured to have a guide strand which is identical to the DNA of interest and a passenger strand that is modified and linked to a molecule for increasing cellular uptake. In particular, a ligand—receptor pair that is particular to neurons would be useful in the current invention.

Another method to administer the DNA to the proper tissue is direct injection/particle bombardment, where the DNA is be injected directly with a syringe and needle into a specific tissue, such as muscle.

An alternative direct injection approach uses particle bombardment (‘gene gun’) techniques: DNA is coated on to metal pellets and fired from a special gun into cells. Successful gene transfer into a number of different tissues has been obtained using this approach. Such direct injection techniques are simple and comparatively safe.

Another method for delivery of DNA to the proper tissue or cell is by using adeno-associated viruses (AAV). DNA delivered in these viral vectors is continually expressed, replacing the expression of the DNA that is not expressed in the subject. Also, AAV have different serotypes allowing for tissue-specific delivery due to the natural tropism toward different organs of each individual AAV serotype as well as the different cellular receptors with which each AAV serotype interacts. The use of tissue-specific promoters for expression allows for further specificity in addition to the AAV serotype.

Other mammalian virus vectors that can be used to deliver the DNA include oncoretroviral vectors, adenovirus vectors, Herpes simplex virus vectors, and lentiviruses.

In particular, HSV vectors are tropic for the central nervous system (CNS) and can establish lifelong latent infections in neurons and thus, are a preferred vector for use in this invention.

Liposomes are spherical vesicles composed of synthetic lipid bilayers which mimic the structure of biological membranes. The DNA to be transferred is packaged in vitro with the liposomes and used directly for transferring the DNA to a suitable target tissue in vivo. The lipid coating allows the DNA to survive in vivo, bind to cells and be endocytosed into the cells. Cationic liposomes (where the positive charge on liposomes stabilize binding of negatively charged DNA), have are one type of liposome.

The DNAs can also be administered with a lipid to increase cellular uptake. The DNA may be administered in combination with a cationic lipid, including but not limited to, lipofectin, DOTMA, DOPE, and DOTAP (such as described in Application No. WO0071096).

Other lipid or liposomal formulations including nanoparticles and methods of administration have been described as for example in U.S. Patent Publication 2003/0203865, 2002/0150626, 2003/0032615, and 2004/0048787. Methods used for forming particles are also disclosed in U.S. Pat. Nos. 5,844,107, 5,877,302, 6,008,336, 6,077,835, 5,972,901, 6,200,801, and 5,972,900.

For certain embodiments, the DNA would be targeted to particular tissues or cells. In a preferred embodiment, the tissue is brain or neurological, and the cells are neurons.

Additionally, the APOE ε4 allele has shown to be associated with disease. Thus, a further embodiment of the present invention is the treatment or prevention of CTE by the administering an agent that binds to the APOE ε4 allele in a subject in need thereof and decreases or prevents the expression of the gene.

A further embodiment of the present invention is a method of treating or preventing CTE by supplying one or the other APOE ε alleles, 1 (SEQ ID NO: 8), 2 (SEQ ID NO: 6) and 3 (SEQ ID NO: 7), which are not associated with disease to a subject in need thereof by any of the methods discussed above.

Lastly, the expression and activity of the Apo E ε4 polypeptide can be also be blocked or decreased in order to treat or prevent CTE.

### EXAMPLES

The present invention may be better understood by reference to the following non-limiting examples, which are presented in order to more fully illustrate the preferred embodiments of the invention. They should in no way be construed to limit the broad scope of the invention.

### Example 1

A cohort of neuropathologically-confirmed CTE patients (n=27, average age=61 years, range=27-84 year, 27 male) was genotyped and the H1 and H2 allele and genotype frequencies compared to a cohort of neuropathologically confirmed non-demented elderly controls (n=52, average age=88 years, range=77-108 years, 19 male and 33 female). All subjects were self-reported Caucasian. The tau haplotype was determined using PCR from genomic DNA isolated from the cerebellum using primers flanking a 238 by deletion (DelIn9) that occurs on the H2 background. This polymorphism has been shown to be an unambiguous tag of H1 and H2 (Baker et al. (1999)).

As shown in Table 1, a significant difference between the CTE and control group was observed, with an elevated H1 allele frequency (0.85) in the CTE patients compared to controls (0.63). The odds ratio of 0.29 suggests that the H2 represents a protective allele.

TABLE 1
MAPT Haplotype Frequency

| | | N (frequency) | | | CTE v. control | | | CTE v. AD | | | AD v. control | | |
|---|---|---|---|---|---|---|---|---|---|---|---|---|---|
| | | CTE | AD | Control | p | OR | CI (95%) | p | OR | CI (95%) | p | OR | CI (95%) |
| Alleles | H1 | 46 (0.85) | 59 (0.72) | 65 (0.63) | 0.003 | 0.29 | 0.12-0.68 | 0.07 | 2.2 | 0.92-5.5 | 0.17 | 0.65 | 0.34-1.2 |
| | H2 | 8 (0.15) | 23 (0.28) | 39 (0.38) | | | | | | | | | |
| Genotype | H1/H1 | 20 (0.74) | 20 (0.49) | 16 (0.31 | 0.0002 | 0.16 | 0.06-0.44 | 0.04 | 0.33 | 0.12-0.96 | 0.07 | 0.47 | 0.20-1.1 |
| | Total | 7 (0.26) | 21 (0.51) | | | | | | | | | | |
| | H2 | | 36 (0.69) | | | | | | | | | | |

Significant association in bold (Chi-squared test)
Total H2 = H1/H2 + H2/H2
CTE—Chronic Traumatic Encephalopathy,
AD—Alzheimer's Disease,
OR—odds ratio,
CI—confidence interval

### Example 2

### Materials and Methods for Examples 3-8

### Subjects

The patients were derived from an ongoing autopsy series at the Center for the Study of Traumatic Encephalopathy. The retrospective assessment of athletic and clinical history has been previously described in Stern et al. (2012), incorporated by reference in its entirety. Post-mortem evaluation of abnormal tau was assessed on formalin-fixed sections immunohistochemically stained using phospho-specific anti-tau monoclonal antisera (pS202, CP13) as described in McKee et al. (2009) and McKee et al. (2013). The stage of CTE disease severity was determined as previously described McKee et al. (2009) and McKee et al. (2013), herein incorporated by reference in its entirety, and shown in FIG. 2.

Briefly, cases with small discrete foci of phospho-tau pathology around blood vessels predominantly at the depths of the sulci in the frontal cortex were categorized as stage I. Cases with more frequent tauopathic foci and spread to the superficial layers of the adjacent cortex were considered stage II. When tau pathology was more widespread, with prominent temporal lobe involvement, including the entorhinal cortex, hippocampal formation and amygdala, the cases were categorized as stage III. When tauopathy was present throughout the telencephalon, with relative preservation of the calcarine cortex, cases were deemed stage IV.

Inclusion criteria were a history of participation in a contact sport, neuropathological confirmation of CTE, Caucasian ancestry and male sex. Subjects without a neuropathological diagnosis of CTE were excluded.

### Genetic Analysis

DNA isolation was from either fresh frozen cerebellum performed as previously described (Santa-Maria et al. (2012)) or formalin-fixed tissue using a QIAamp DNA FFPE kit (Qiagen, Valencia, Calif.) according to the manufacturer's instructions. To classify population ancestry, a set of 100 unlinked single-nucleotide polymorphisms (SNPs) were used as previously described (Janicki et al. (2013)). The ancestry for each subject was estimated by comparing the frequency of 100 single nucleotide polymorphisms from 90 Asian, 60 African and 60 Caucasians using the program STRUCTURE (Pritchard et al. (2000); Falush et al. (2003)). The MAPT haplotype was determined using two haplotype tagging SNPs (rs9864 and rs1800547) on the Sequenom iPlex platform as previously described (Santa-Maria et al. 2012). Discrepancies were resolved using a PCR-based genotyping of the DelIn9 polymorphism as described by Baker et al. (1999). APOE genotype was determined as previously described in McKee et al. (2013). To estimate MAPT haplotype and APOE allele frequencies in the population, the genotypes from the 1000 genomes project website were obtained (Consortium TGP (2010)).

### Statistical Analysis

Statistical analyses were performed in Graphpad Prism. The statistical significance of differences between means were evaluated by Student's t-test performed as unpaired, two-tailed distribution of arrays and presented as p values. Frequency comparisons were performed using a Fisher's exact test.

### Example 3

### Patient Data

A total of 36 male athletes with neuropathologically confirmed CTE were studied (Table 2). The mean age was 61.4±3.2 years (range=27 to 98). Nine of these individuals had a history of playing two sports, and one had a history of playing three sports. Of these, 87% (n=30) had a history of playing American football (mean=12.7±1.0 years, range=3-21 years), 53% professionally (n=19, mean=6.7±0.8 years, range=1-15 years). 14% (n=5) had a history of playing ice hockey (mean=20.5±3.9 years, range=7-30 years), 11% professionally (n=4, mean=10.6±3.4 years, range=1-15 years). 14% had a history of boxing (n=5, mean=13.3±8.2 years, range=1-37 years), 3% professionally (n=1, 13 years). Finally, there was one professional wrestler (years=7 years; professional years=2 years) and one semi-professional soccer player (years=22 years). 23% (n=8) had a military history and 85% (n=29) had a known history of a concussion.

At autopsy, all subjects exhibited the distinctive tauopathic changes of CTE. These neuropathological findings were used to determine the disease stage McKee et al. (2013). In this cohort, 5.6% (n=2) were stage I, 27.78% (n=10) stage II, 25.0% (n=9) stage III and 41.67% (n=15) stage IV (Table 2; see also FIG. 2). The mean age of death correlated positively with CTE stage (Table 2). Eleven (11) cases were diagnosed with one additional neuropathological disease, and four with two. These diagnoses were motor neuron disease (n=5), Alzheimer's disease (n=5), Parkinson's disease (n=5), diffuse Lewy body disease (n=3), and FTLD TPD-43 (n=1).

All 36 subjects were Caucasian as determined by family report. To account for hidden ethnic stratification, the ancestry of the subjects was determined using a set of single nucleotide polymorphisms (SNPs) that served as ancestry markers Janicki et al. (2013). Comparison with known Asian (n=90), Caucasian (n=60) and African (n=60) reference individuals reveals that the average proportion of the genome in these CTE patients derived from the ancestral Caucasian population is 0.98+/−0.003 (range 0.90-0.99). This analysis confirmed the relative genetic homogeneity of these patients.

TABLE 2
Summary of patient data

| | Total | Stage I | Stage II | Stage | Stage |
|---|---|---|---|---|---|
| | (n) | (n) | (n) | III (n) | IV (n) |
| | Mean age of death ± SEM, yr | | | | |
| | 61.4 ± | 33 ± | 46.7 ± | 61.1 ± | 75.1 ± |
| | 3.12 (36) | 1.00 (2) | 6.01 (10) | 4.07 (9) | 2.94 (15) |
| Football % | 83% (30) | 100% (2) | 60% (6) | 100% | 87% |
| | | | | (9) | (13) |
| Ice hockey % | 14% (5) | 0% (0) | 40% (4) | 11% (1) | 0% (0) |
| Boxing % | 11% (4) | 0% (0) | 0 (0) | 11% (1) | 20% (3) |
| Pro wrestling % | 3% (1) | 0% (0) | 10% (1) | 0% (0) | 0% (0) |
| Other sport % | 17% (6) | 50% (1) | 30% (3) | 0% (0) | 13% (2) |
| Military | 22% (8) | 0% (0) | 10% (1) | 0% (0) | 47% (7) |
| veteran % | | | | | |
| Concussion | 81% (29) | 50% (1) | 100% | 89% (8) | 67% (10) |
| history % | | | (10) | | |
| CTE | 89% (32) | 100% (2) | 90% (9) | 100% | 87% (13) |
| symptoms % | | | | (9) | |
| Dementia % | 47% (17) | 0% (0) | 10% (1) | 33% (3) | 87% (13) |
| Parkinsonism % | 3% (1) | 0% (0) | 0% (0) | 11% (1) | 0% (0) |
| Movement | 31% (11) | 50% (1) | 20% (2) | 11% (1) | 47% (7) |
| disorder % | | | | | |
| Motor neuron | 14% (5) | 50% (1) | 20% (2) | 11% (1) | 7% (1) |
| disease % | | | | | |
| Other neuro- | 42% (15) | 50% (1) | 30% (3) | 44% (4) | 47% (7) |
| pathologic | | | | | |
| diagnoses % | | | | | |

CTE = chronic traumatic encephalopathy,
SEM = standard error of the mean,
pro = professional

### Example 4

### MAPT Haplotype H1 and APOE ε4 Allele were More Frequent in Subjects with CTE

To ask whether the tau gene is associated with CTE, the MAPT haplotype in this cohort was determined and compared the frequencies to population controls. An 80.6% allele frequency for H1 (n=58) and 19.4% for H2 (n=14) was found in the cohort. The frequency of H1/H1 homozygotes was 69.4% (n=25), H1/H2 heterozygote frequency was 22.2% (n=8), and H2/H2 homozygotes were the least common with 8.3% frequency (n=3).

Comparison of the MAPT haplotype frequency between these CTE cases (n=36) and male population controls obtained from the 1000 genomes project (n=143) shows an elevation of H1 in CTE (p=0.071 and 0.21 for genotypes and alleles respectively (Table 3) (Consortium TGP (2010)). A significant elevation in the APOE ε4 allele frequency in the CTE patients as compared to controls was also found (p=0.047, Table 3). Together, these results support the hypothesis that MAPT haplotype, as has been proposed for the APOE ε4 allele, increases susceptibility to CTE.

TABLE 3
MAPT haplotype and APOE ε4 allele frequencies in CTE v. population controls

| | | Genotypes | | | | | | Alleles | | | | |
|---|---|---|---|---|---|---|---|---|---|---|---|---|
| | | H1/H1 | H1/H2 | H2/H2 | | | CI | H1 | H2 | | | CI |
| | n | (freq) | (freq) | (freq) | p* | OR | (95%) | (freq) | (freq) | p | OR | (95%) |
| 1000 | 143 | 77 (0.54) | 56 (0.39) | 10 (0.07) | 0.091 | 1.95 | 0.89-4.26 | 210 (0.73) | 76 (0.27) | 0.21 | 1.5 | 0.79-2.84 |
| genomes | | | | | | | | | | | | |
| CTE | 36 | 25 (0.69) | 8 (0.22) | 3 (0.08) | | | | 58 (0.81) | 14 (0.19) | | | |
| | | Genotypes | | | | | | | | | | |
| | | | | Non-ε4/ | | | | Alleles | | | | |
| | | ε4/ε4 | ε4/Non-ε4 | Non-ε4 | | | CI | ε4 | Non-ε4 | | | |
| | n | (freq) | (freq) | (freq) | p* | OR | (95%) | (freq) | (freq) | p | OR | CI (95%) |
| 1000 | 143 | 2 (0.01) | 33 (0.23) | 108 (0.76) | 0.159 | 1.74 | 0.80-3.80 | 37 (0.13) | 249 (0.87) | 0.047 | 1.92 | 1.00-3.70 |
| genomes | | | | | | | | | | | | |
| CTE | 36 | 3 (0.08) | 10 (0.28) | 23 (0.64) | | | | 16 (0.22) | 56 (0.78) | | | |

MAPT = microtubule-associated protein tau gene,
ε4 = APOE ε4 allele,
freq = frequency,
*significant associations in bold (chi-squared test, H1/H1 v. total H2 carriers or total ε4 v. non-ε4)

### Example 5

### No Association Between MAPT Haplotype and APOE Genotype and mTBI History

Next, it was asked whether MAPT haplotype is associated with mTBI history. Most of the patients in this cohort have a known history of concussion. However, whether this represents a reliable measure of mTBI remains unclear, as clinically silent, repetitive subconcussive brain injury may contribute (Bailes et al. (2013); Koerte et al. (2012)).

There is no observed significant difference in the total number of concussions between H1 and H2 carriers (Table 3).

The athletic history (i.e., American football, ice hockey, boxing and professional wrestling) between H1 and H2 carriers was compared, and no significant difference in the frequency of haplotypes by participation or the years of play in these sports was found (Table 4). Eight patients were military veterans but there was also no significant difference in the military history or combat history between H1 and H2 carriers found.

It was also asked whether APOE ε4 allele frequency was associated with mTBI history. As with MAPT, there is no significant difference between APOE ε4 carriers and non-APOE ε4 CTE patients with respect to concussion, athletic or military history (Table 5). Together, these results indicate that neither MAPT haplotype nor APOE genotype are associated with differences in mTBI history.

TABLE 4
mTBI history between MAPT H1 and H2 carriers in CTE (n = 36)

| | H1 | | | H2 | | | | | | |
|---|---|---|---|---|---|---|---|---|---|---|
| | | Mean ± SEM | | | Mean ± SEM | | H1 | H2 | | |
| Variable | N | (range) | | n | (range) | p* | Freq (+/−) | Freq (+/−) | | p ** |
| Concussion (y/n) | | | | | | | 0.88 (21/3) | 0.8 | (8/2) | 0.62 |
| Concussion # | 21 | 19.4 ± 5.8 | (0-100) | 9 | 16 ± 10.7 (0-100) | 0.76 | | | | |
| FB (y/n) | | | | | | | 0.79 (19/5) | 1.00 | (11/0) | 0.16 |
| FB (yr) | 19 | 12.6 ± 1.4 | (3-21) | 9 | 12.9 ± 1.14 (6-17) | 0.89 | | | | |
| FB-pro (yr) | 12 | 7.2 ± 1.7 | (1-15) | 7 | 5.86 ± 1.30 (1-10) | 0.46 | | | | |
| HK (y/n) | 4 | | | 1 | | | 0.17 (4/20) | 0.11 | (1/8) | 1.00 |
| HK (yr) | 4 | 23.9 ± 2.5 | (18-30) | 1 | NA | NA | | | | |
| HK-pro (yr) | 4 | 13.3 ± 2.8 | (6-18) | 1 | NA | NA | | | | |
| BX (y/n) | 4 | | | 0 | | | 0.17 (4/20) | 0 | (0/10) | 0.30 |
| BX (yr) | 4 | 13.3 ± 8.2 | (1-37) | 0 | NA | NA | | | | |
| BX-pro (yr) | 4 | 3.3 ± 3.3 | (0-13) | 0 | NA | NA | | | | |
| PW (y/n) | 1 | | | 0 | | | 0.04 (1/23) | 0 | (0/10) | 1.00 |
| PW (yr) | 1 | 7 | | 0 | NA | NA | | | | |
| PW-pro (yr) | 1 | 2 | | 0 | NA | NA | | | | |
| Other sport (y/n) | 5 | | | 1 | | | 0.21 (5/19) | 0.1 | (1/9) | 0.64 |
| Other sport (yr) | 4 | 9.5 ± 4.3 | (2-22) | 1 | NA | NA | | | | |
| Other sport, pro (yr) | 4 | 0 ± 0 | (0-0) | 1 | NA | NA | | | | |
| Military (y/n) | | | | | | | 0.20 (5/20) | 0.30 | (3/7) | 0.66 |
| Combat (y/n) | | | | | | | 0.25 (1/3) | 0.33 | (1/2) | 1 |
| Concussion, combat (y/n) | | | | | | | 0.25 (1/3) | 0.33 | (1/2) | |

MAPT = microtubule-associated protein tau,
H1 = H1/H1 homozygotes,
H2 = H1/H2 + H2/H2,
CTE = chronic traumatic encephalopathy,
SEM = standard error of the mean,
Freq = frequency,
*= Student's t-test,
** = Fisher's exact test, significant values in bold,
FB = American football,
HK = ice hockey,
BX = boxing,
PW = professional wrestling,
pro = professional

TABLE 5
Clinical and Neuropathological Findings in MAPT H1 and H2 Carriers in CTE (n = 36)

| | Non-APOE ε4 | | | APOE ε4 | | | | | | |
|---|---|---|---|---|---|---|---|---|---|---|
| | | Mean ± SEM | | | Mean ± SEM | | | Non-APOE ε4 | APOE ε4 | |
| Variable | n | (range) | | n | (range) | | p * | Freq (+/−) | Freq (+/−) | p ** |
| Concussion (y/n) | | | | | | | | 0.86 (18/3) | 0.85 (11/2) | 1.00 |
| Concussion # | 19 | 15.8 ± 5.5 | (0-100) | 11 | 22.9 ± 10.3 | (0-100) | 0.51 | | | |
| FB (y/n) | | | | | | | | 0.91 (20/2) | 0.77 (10/3) | 0.337 |
| FB (yr) | 18 | 12.6 ± 1.3 | (3-21) | 10 | 13.0 ± 1.7 | (4-19) | 0.85 | | | |
| FB-pro (yr) | 12 | 6.8 ± 1.1 | (1-15) | 7 | 6.6 ± 1.3 | (1-11) | 0.92 | | | |
| HK (y/n) | | | | | | | | 0.15 (3/17) | 0.15 (2/11) | 1.00 |
| HK (yr) | 3 | 18.2 ± 5.6 | (7-24) | 2 | 24 ± 6 | (18-30) | 0.54 | | | |
| HK-pro (yr) | 3 | 7.7 ± 5.0 | (0-17) | 2 | 15 ± 3 | (12-18) | 0.36 | | | |
| BX (y/n) | | | | | | | | 0.10 (2/19) | 0.15 (2/11) | 0.627 |
| BX (yr) | 2 | 6.0 ± 5.0 | (1-11) | 2 | 20.5 ± 16.5 | (4-37) | 0.49 | | | |
| BX-pro (yr) | 2 | NA | | 2 | 6.5 ± 6.5 | (0-13) | 0.42 | | | |
| PW (y/n) | | | | | | | | 0.05 (1/20) | 0 (0/13) | 1.00 |
| PW (yr) | NA | | | NA | 7 | | NA | | | |
| PW-pro (yr) | NA | | | NA | 2 | | NA | | | |
| Other sport (y/n) | | | | | | | | 0.14 (3/18) | 0.23 (3/10) | 0.653 |
| Other sport (yr) | 3 | 5 ± 1.5 | (2-7) | 2 | 14.5 ± 7.5 | (7-22) | 0.21 | | | |
| Other sport, pro (yr) | 3 | 0 ± 0 | (0-0) | 2 | 0 ± 0 | (0-0) | | | | |
| Military (y/n) | | | | | | | | 0.27 (6/16) | 0.15 (2/11) | 0.680 |
| Combat (y/n) | | | | | | | | 0.40 (2/3) | 0 (0/2) | 0.524 |
| Concussion, combat (y/n) | | | | | | | | 0.40 (2/3) | 0 (0/2) | NA |

APOE = apolipoprotein E gene,
CTE = chronic traumatic encephalopathy,
SEM = standard error or the mean,
Freq = frequency,
* = Student's t-test,
** = Fisher's exact test,
FB = American football,
HK = ice hockey,
BX = boxing,
PW = professional wrestling,
pro = professional

### Example 6

### CTE Patients with the MAPT HI Haplotype have a More Rapid Clinical Course

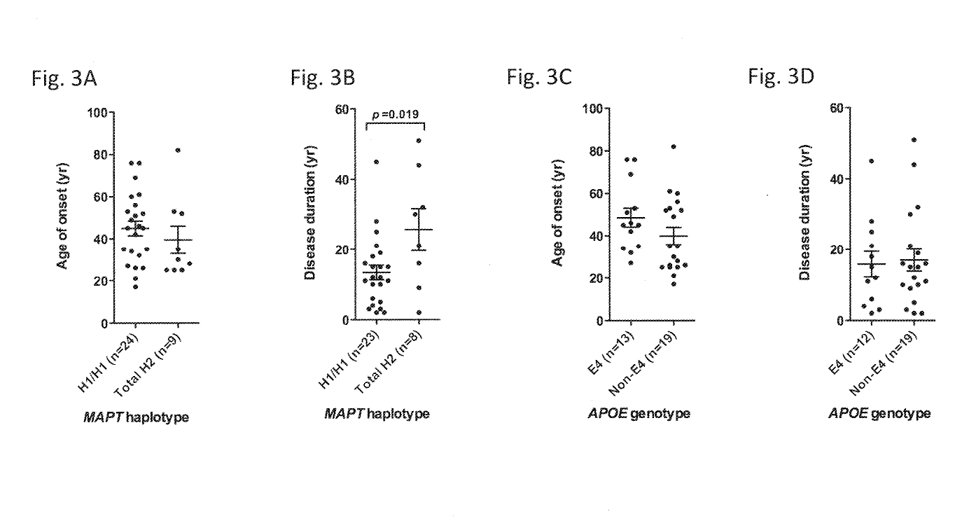

Next, it was asked whether there was a difference in the clinical findings between MAPT H1 and H2 carriers. While there was no difference in the age of onset of clinical symptoms. between H1 and H2 carriers (FIG. 3A), there was a statistically significant decrease in the disease duration (P=0.019) (i.e., age of death minus age of symptom onset) in H1 homozygotes compared to H2 carriers (FIG. 3B, Table 6). The average disease duration±standard error of the mean was 13.4±2.1 years for H1 homozygotes, and 25.6±6.0 years for H2 carriers (difference=12.2±4.8 yr, 95% confidence interval=2.1 to 22.3, R squared=0.17).

Patients with CTE may exhibit depression, suicidality, dementia, parkinsonism and motor neuron disease. There was no significant difference in the frequency of these symptoms between the H1 and H2 carriers.

The average age of death was lower in H1 homozygotes (n=25, 58.8±3.6 years, range=27-94) compared to H2 carriers (n=11, 67.4±6.5 years, range=32-98), but this does not reach statistical significance (difference=8.6±6.9; (p=0.22).

There was no difference in disease duration or age of onset between CTE patients that carry the APOE ε4 allele, and those who do not (FIGS. 3C and 3D).

Together, these findings suggest that CTE patients who are homozygous for H1 have a more rapid clinical course than H2 carriers.

TABLE 6
Clinical and neuopathological findings in MAPT H1 and H2 carriers in CTE (n = 36)

| | H1 | | | H2 | | | | | | | |
|---|---|---|---|---|---|---|---|---|---|---|---|
| | | Mean ± SEM | | | Mean ± SEM | | | H1 | | H2 | |
| Variable | n | (range) | | n | (range) | | p* | Freq (+/−) | | Freq (+/−) | p** |
| Age of death (yr) | 25 | 58.8 ± 3.6 | (27-94) | 11 | 67.3 ± 6.5 | (32-98) | 0.220 | | | | |
| CTE Sx (Y/N) | | | | | | | | 1 | (24/0) | 0.89 (8/1) | 0.27 |
| Age CTE Sx (yr) | 24 | 45.0 ± 3.4 | (17-76) | 9 | 39.4 ± 6.5 | (25-82) | 0.429 | | | | |
| Disease duration (yr) | 23 | 13.4 ± 2.1 | (2-45) | 8 | 25.6 ± 6.0 | (2-51) | 0.019 | | | | |
| Dementia (Y/N) | | | | | | | | 0.5 | (12/12) | 0.5 (5/5) | 1.000 |
| Movement disorder (Y/N) | | | | | | | | 0.26 | (6/17) | 0.5 (5/5) | 0.240 |
| Parkinsonism (Y/N) | | | | | | | | 0.04 | (1/22) | 0 (0/9) | 1.000 |
| MND (Y/N) | | | | | | | | 0.17 | (4/20) | 0.1 (1/9) | 1.000 |
| CTE NP Stage | 25 | 3.04 ± 0.17 | (2-4) | 11 | 3 ± 0.38 | (1-4) | 0.911 | | | | |
| Other NP Dx (Y/N) | | | | | | | | 0.5 | (12/12) | 0.3 (3/7) | 0.45 |
| CTE NP Stage/age of | 25 | 0.054 ± 0.003 | (0.034-0.075) | 11 | 0.044 ± 0.004 | (0.023-0.066) | 0.034 | | | | |
| death ratio (yr⁻¹) | | | | | | | | | | | |

MAPT = microtubule-associated protein tau,
H1 = H1/H1 homozygotes,
H2 = H1/H2 + H2/H2,
CTE = chronic traumatic encephalopathy,
SEM = standard error of the mean,
*= Student's t-test,
**= Fisher's exact test, significant values in bold,
MND = Motor neuron disease

### Example 7

### MAPT H1 Haplotype is Associated with Progression of Taupathic Changes in CTE

There is hierarchical progression that can serve as the basis for a four-tiered staging system (McKee et al. (2013)). See also FIG. 2.

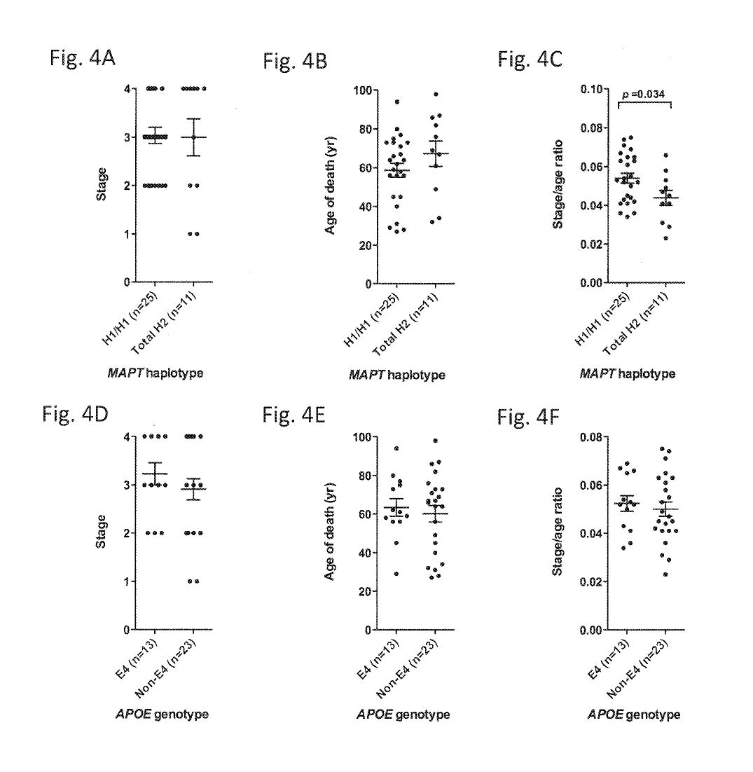

It was found that the average CTE stage was higher in H1 homozygotes (n=25, 3.04±0.17 yr⁻¹) as compared to H2 carriers (n=11, 3.00±0.91 yr⁻¹), but this is not statistically significant (FIG. 4A, Table 7). Further, the average age of death was higher in H2 carriers, but this increase is not significant (FIG. 4B). Comparison of the ratio of the CTE stage to age of death, which was adjusted for the correlation between these variables, revealed a significant increase in the H1 homozygotes (n=25, 0.054±0.003 yr⁻¹) compared to H2 carriers (n=11, 0.044±0.004 y⁻¹, p=0.034) (FIG. 4C).

In contrast, APOE ε4 carrier status was not associated with age of death, duration of CTE symptoms, frequency of dementia, parkinsonism, motor neuron disease, CTE stage, or CTE stage to age of death ratio (FIGS. 4D-F, Table 7). There was also no difference in the presence of TDP-43 positive inclusions, Lewy bodies, or amyloid deposition between MAPT H1 homozygotes and H2 carriers.

Together these findings indicate that MAPT haplotype influences the rate of progression of tauopathic changes in CTE.

TABLE 7
Clinical and neuropathological findings in APOE ε4 and non-ε4 carriers in CTE

| | Non-APOE ε4 | | | APOE ε4 | | | | | | | |
|---|---|---|---|---|---|---|---|---|---|---|---|
| | | Mean ± SEM | | | Mean ± SEM | | | Non-APOE ε4 | APOE ε4 | | |
| Variable | n | (range) | | n | (range) | | p* | Freq (+/−) | Freq (+/−) | | p** |
| Age of death (yr) | 23 | 60.2 ± 4.3 | (27-98) | 13 | 63.5 ± 4.6 | (29-94) | 0.63 | | | | |
| CTE Symptoms (Y/N) | | | | | | | | 0.95 (19/1) | 1 | (13/0) | 1.00 |
| Age CTE Sx (yr) | 19 | 40.4 ± 4.1 | (17-82) | 13 | 48.5 ± 4.5 | (27-76) | 0.21 | | | | |
| Disease duration (yr) | 18 | 17.8 ± 3.2 | (2-51) | 12 | 15.8 ± 3.6 | (2-45) | 0.69 | | | | |
| Dementia (Y/N) | | | | | | | | 0.48 (10/11) | 0.54 | (7/6) | 1.00 |
| Movement disorder | | | | | | | | 0.40 (8/12) | 0.23 | (3/10) | 0.46 |
| (Y/N) | | | | | | | | | | | |
| Parkinsonism (Y/N) | | | | | | | | 0.05 (1/18) | 0 | (0/13) | 1.00 |
| MND (Y/N) | | | | | | | | 0.14 (3/18) | 0.15 | (2/11) | 1.00 |
| CTE NP Stage | 23 | 2.91 ± 0.22 | (1-4) | 13 | 3.23 ± 0.231 | (2-4) | 0.35 | | | | |
| Other NP diagnosis | | | | | | | | 0.38 (8/13) | 0.54 | (7/6) | 0.48 |
| (Y/N) | | | | | | | | | | | |
| CTE NP Stage/age of | 23 | 0.050 ± 0.003 | (0.023-0.075) | 13 | 0.052 ± 0.003 | (0.034-0.069) | 0.62 | | | | |
| death ratio | | | | | | | | | | | |

APOE = apolipoprotein E gene,
CTE = chronic traumatic encephalopathy,
SEM = standard error of the mean,
Freq = frequency,
*= Student's t-test, two tails,
**= Fisher's exact test,
MND = motor neuron disease

### Example 8

### Analysis of Football Players Only

Finally, it was important to consider the possibility that participation in specific sports might independently influence the findings. This cohort consisted of athletes with a heterogeneous mixture of sports histories, but 26 individuals had American football as their primary sport, 19 of whom had played professionally. When the analysis was restricted to football players alone, the disease duration is significantly lower in H1 homozygotes compared to H2 carriers in all football players (p=0.035) as well as professional players (p=0.025; Table 8). The stage/age ratio was also increased in H1 homozygotes compared to H2 carriers among all football players as well as professionals (Table 8).

TABLE 8
Post-hoc analysis of MAPT haplotypes in football players with CTE

| Football, total | | Football, professional | |
|---|---|---|---|
| n | Mean ± SEM (range) | n | Mean ± SEM (range) |

CTE NP stage/age of death ratio

| | | | | |
|---|---|---|---|---|
| H1 | 16 | 0.053 ± 0.002 (0.036-0.074) | 12 | 0.051 ± 0.002 (0.036-0.067) |
| H2 | 10 | 0.044 ± 0.004 (0.023-0.066) | 7 | 0.043 ± 0.004 (0.023-0.058) |
| p | | 0.075 | | 0.101 |

Disease duration (yr)

| | | | | |
|---|---|---|---|---|
| H1 | 15 | 12.6 ± 2.12 (3-45) | 11 | 13.6 ± 2.4 (3-45) |
| H2 | 7 | 26.3 ± 6.4 (2-51) | 6 | 30.3 ± 5.05 (9-51) |
| p | | 0.035 | | 0.025 |

MAPT = microtubule-associated protein tau,
CTE = chronic traumatic encephalopathy,
H1 = Hl/H1 homozygotes, H2 = Hl/H2 + H2/H2,
SEM = standard error of the mean, comparisons made using a Student's t-test, significant values in bold

### REFERENCES

Abraham et al. (2009) American Journal of Medical Genetics Neuropsychiatry Genetics 150B:1152-55
Adams et al. (2010) PLoS One 5 e10810
Aronov et al. (1999) Journal of Molecular Neuroscience 12:131-45
Aronov et al. (2001) The Journal of Neuroscience 21:6577
Bailes et al. (2013) Journal of Neurosurgery 119:1235-45
Baker et al. (1999) Human Molecular Genetics 8:711-15
Bekris et al. (2010) Journal of Geriatric Psychiatry and Neurology 23:228-42
Boland et al. (2008) The Journal of Neuroscience 28:6926
Buratti et al. (2010) The FEBS Journal 277:2268
Consortium TGP (2010) Nature 467:1061-73.
Corsellis et al. (1973) Psychological Medicine 3(3):270-303
DeKosky et al. (2007) Archives of Neurology 64(4):541-4
DiMaria et al. (2000) Annals of Neurology 47:374-377
Falush et al. (2003) Genetics 164:1567-87
Gandy et al. (2012) Science Translational Medicine 4(134):134 ed4
Gavett et al. (2011) Clinics in Sports Medicine 30(1):179-88
Gentleman et al. (1993) Neuroscience Letters 160:139
Goldstein et al. (2012) Science Translational Medicine 4(134):134ra60
Hayesmoore et al. (2009) Neurobiological Aging 30:1652-56
Janicki et al. (2013) Dementia and Geriatric Cognitive Disorders 35:340-6
Janocko et al. (2012) Acta Neuropathologica 124(5):681-92
Koerte et al. (2012) The Journal of the American Medical Association 308:1859-61
Maniatis et al. (1982) Sambrook et al. (1989) (1989) Molecular Cloning: A Laboratory Manual (Cold Spring Harbor Laboratory, 2nd Ed., Cold Spring Harbor, N.Y.
Mayuex et al. (1995) Neurology 45(3):555-7
McKee et al. (2008) Journal of Neuropathology and Experimental Neurology 67(5):500
McKee et al. (2009) Journal of Neuropathology and Experimental Neurology 68(7):709-35
McKee et al. (2010) Journal of Neuropathology and Experimental Neurology 69(9):918-29
McKee et al. (2013) Brain 136:43-64 (first published online Dec. 2, 2012)
Myers et al. (2005) Neurobiology Disease 25: 561-570
Neumann et al. (2006) Science 314(5796):130-3.
Nonaka et al. (2010) The Journal of Biological Chemistry 285:34885
Omalu et al. (2005) Neurosurgery 57(1):128-34
Omalu et al. (2006) Neurosurgery 59(5):1086-92
Omalu et al. (2010) Journal of Forensic Nursing 6(1):40-6
Omalu et al. (2011a) Neurosurgical Focus 1(5):E3
Omalu et al. (2011b) Neurosurgery 69(1):173-83
Pritchard et al. (2000) Genetics 155:945-59
Roberts et al. (1990) Journal of Neurology, Neurosurgery, and Psychiatry 53(5):373-8
Saman et al. (2012) The Journal of Biological Chemistry 287:3842
Sambrook et al. (1989) Molecular Cloning: A Laboratory Manual (Cold Spring Harbor Laboratory, 2nd Ed., Cold Spring Harbor, N.Y.
Santa-Maria et al. (2012) Acta Neuropathologica 124:693-704
Stefansson et al. (2005) Nature Genetics 37: 129-37
Stern et al. (2011) PM R 3(10 Suppl 2):5460-7
Stern et al. (2013) Neurology 81:1121-29
Tierney (2013) New York Times, Dec. 17, 2013
Trabzuni et al. (2012) Human Molecular Genetics 21(18):4094-103
Vandrovcova et al. (2010) Current Alzheimer Research 7: 726-34
Wade-Martins et al. (2012) Nature Reviews. Neurology 8:477-478
Wider et al. (2012) Journal of Neurology, Neurosurgery, and Psychiatry 83:424-29
Zody et al. (2008) Nature Genetics 40(9):1076-83

1. A method of screening, diagnosing, predicting or identifying chronic traumatic encephalopathy in a subject, comprising:
a. obtaining biological tissue or bodily fluid from the subject;
b. isolating and purifying a sample of nucleic acid from the biological tissue or bodily fluid; and
c. detecting the presence of Apoliprotein E allele ε4 in the sample of nucleic acid by sequencing the nucleic acid sample obtained from the biological tissue or bodily fluid of the subject, and comparing the sequence of the nucleic acid sample to the known reference nucleic acid sequence of Apoliprotein allele ε4,
wherein the presence of Apoliprotein E allele ε4 determines, diagnoses, predicts or identifies the subject as having chronic traumatic encephalopathy.

2. The method of claim 1, wherein the subject is human.

3. The method of claim 1, wherein the subject is in the military or is entering the military.

4. The method of claim 1, wherein the subject plays a sport.

5. The method of claim 4, wherein the sport is selected from the group consisting American football, boxing, ice hockey, wrestling, baseball, cycling, skiing, ski jumping, snowboarding, snowmobiling, bobsledding, luge, ice skating, roller blading, roller skating, inline skating, skateboarding, scooter riding, soccer, basketball, field hockey, softball, water sports, use of powered recreational vehicles, horseback riding, cheerleading, dancing, gymnastics, golf, trampolines, rugby, and lacrosse.

6. The method of claim 1, wherein the subject has suffered one or more traumatic brain injuries.

7. The method of claim 1, wherein the biological tissue is brain or epidermis.

8. The method of claim 1, wherein the bodily fluid is cerebrospinal fluid, saliva, whole blood, buffy coat, serum, plasma, sweat or urine.

9. The method of claim 1, wherein the nucleic acid is RNA, cDNA or genomic DNA.

10. The method of claim 1, wherein the presence of the Apolipoprotein allele ε4 is detected by amplifying the Apolipoprotein E gene in the sample of nucleic acid from the biological tissue or bodily fluid of the subject with a primer.

11. The method of claim 1, wherein the sequence of the nucleic acid sample of the subject is compared to the reference nucleic acid sequence of SEQ ID NO: 1, SEQ ID NO:6, SEQ ID NO: 7, or SEQ ID NO: 8.

12. A method of screening, diagnosing, predicting or identifying chronic traumatic encephalopathy in a subject, comprising:
a. obtaining biological tissue or bodily fluid from the subject;
b. isolating and purifying a sample of nucleic acid from the biological tissue or bodily fluid; and
c. detecting the presence of Apoliprotein E allele ε4 in the sample of nucleic acid;
wherein the presence of the Apoliprotein E allele ε4 in the sample of nucleic acid is detected by an assay selected from the group consisting of (a) hybridizing a Apoliprotein E allele ε4 gene probe to the nucleic acid sample, and detecting the presence of hybridization products, (b) hybridizing an allele-specific probe to nucleic acid sample and detecting the presence of hybridization products in the sample, (c) amplifying all or part of the Apo E allele from the nucleic acid sample to produce an amplified sequence and sequencing the amplified sequence, (d) amplifying all or part of the Apo E allele from the nucleic acid sample using primers for a specific Apo E allele ε4 and determining the presence of a hybridization product in the sample, (e) amplifying all or part of the Apo E allele from the nucleic acid sample using primers for a specific Apo E allele ε4 and determining the presence of amplicons in the sample, (f) molecularly cloning all or part of the Apo E allele from the nucleic acid sample to produce a cloned sequence and sequencing the cloned sequence, (g) amplification of Apo E allele sequences in the nucleic acid sample and hybridization of the amplified sequences to nucleic acid probes which comprise the specific Apo E allele ε4 sequence, and (h) in situ hybridization of the Apo E allele of the nucleic acid sample with nucleic acid probes which comprise the Apo E allele ε4; and wherein the presence of Apoliprotein E allele ε4 determines, diagnoses, predicts or identifies the subject as having chronic traumatic encephalopathy.

13. The method of claim 12, wherein the subject is human.

14. The method of claim 12, wherein the subject is the military or is entering the military.

15. The method of claim 12, wherein the subject plays a sport.

16. The method of claim 15, wherein the sport is selected from the group consisting American football, boxing, ice hockey, wrestling, baseball, cycling, skiing, ski jumping, snowboarding, snowmobiling, bobsledding, luge, ice skating, roller blading, roller skating, inline skating, skateboarding, scooter riding, soccer, basketball, field hockey, softball, water sports, use of powered recreational vehicles, horseback riding, cheerleading, dancing, gymnastics, golf, trampolines, rugby, and lacrosse.

17. The method of claim 12, wherein the subject has suffered one or more traumatic brain injuries.

18. The method of claim 12, wherein the biological tissue is brain or epidermis.

19. The method of claim 12, wherein the bodily fluid is cerebrospinal fluid, saliva, whole blood, buffy coat, serum, plasma, sweat or urine.

20. The method of claim 12, wherein the nucleic acid is RNA, cDNA or genomic DNA.

21. A method of screening, diagnosing, prognosing, predicting or identifying chronic traumatic encephalopathy in a subject, comprising:
a. obtaining biological tissue or bodily fluid from the subject;
b. isolating and purifying a sample of nucleic acid from the biological tissue or bodily fluid; and
c. detecting the presence of H1 haplotype of the microtubule-associated protein tau (MAPT) locus in the sample of nucleic acid by sequencing the nucleic acid sample obtained from the biological tissue or bodily fluid of the subject, and comparing the sequence of the nucleic acid sample to the known reference nucleic acid sequences of the H1 haplotype of the MAPT locus,
wherein the presence of H1 haplotype of the MAPT locus determines, diagnoses, predicts or identifies the subject as having chronic traumatic encephalopathy and/or prognoses that the subject will have a more rapid clinical decline from chronic traumatic encephalopathy.

22. The method of claim 21, wherein the subject is human.

23. The method of claim 21, wherein the subject is the military or is entering the military.

24. The method of claim 21, wherein the subject plays a sport.

25. The method of claim 24, wherein the sport is selected from the group consisting American football, boxing, ice hockey, wrestling, baseball, cycling, skiing, ski jumping, snowboarding, snowmobiling, bobsledding, luge, ice skating, roller blading, roller skating, inline skating, skateboarding, scooter riding, soccer, basketball, field hockey, softball, water sports, use of powered recreational vehicles, horseback riding, cheerleading, dancing, gymnastics, golf, trampolines, rugby, and lacrosse.

26. The method of claim 21, wherein the subject has suffered one or more traumatic brain injuries.

27. The method of claim 21, wherein the biological tissue is brain, or epidermis.

28. The method of claim 21, wherein the bodily fluid is cerebrospinal fluid, saliva, whole blood, buffy coat, serum, plasma, sweat or urine.

29. The method of claim 21, wherein the nucleic acid is RNA, cDNA or genomic DNA.

30. The method of claim 21, wherein the presence of the H1 haplotype of the MAPT locus is detected by amplifying the H1 haplotype of the MAPT locus in the sample of nucleic acid from the biological tissue or bodily fluid of the subject with a primer.

31. The method of claim 21, wherein the sequence of the nucleic acid sample of the subject is compared to the reference nucleic acid sequence of SEQ ID NO: 2, SEQ ID NO: 3, SEQ ID NO: 4, or SEQ ID NO: 5.

32. A method of screening, diagnosing, pronosing, predicting or identifying chronic traumatic encephalopathy in a subject, comprising:
a. obtaining biological tissue or bodily fluid from the subject;
b. isolating and purifying a sample of nucleic acid from the biological tissue or bodily fluid; and
c. detecting the presence of H1 haplotype of the microtubule-associated protein tau (MAPT) locus in the sample of nucleic acid;
wherein the presence of the H1 haplotype of the MAPT locus in the sample of nucleic acid is detected by an assay selected from the group consisting of (a) hybridizing a H1 haplotype probe to the nucleic acid sample, and detecting the presence of hybridization products, (b) hybridizing an allele-specific probe to nucleic acid sample and detecting the presence of hybridization products in the sample, (c) amplifying all or part of the MAPT locus from the nucleic acid sample to produce an amplified sequence and sequencing the amplified sequence, (d) amplifying all or part of the MAPT locus from the nucleic acid sample using primers for the H1 haplotype of the MAPT locus and determining the presence of a hybridization product in the sample, (e) amplifying all or part of the MAPT locus from the nucleic acid sample using primers for the H1 haplotype and determining the presence of amplicons in the sample, (f) molecularly cloning all or part of the MAPT locus from the nucleic acid sample to produce a cloned sequence and sequencing the cloned sequence, (f) amplification of MAPT locus sequences in the nucleic acid sample and hybridization of the amplified sequences to nucleic acid probes which comprise the H1 haplotype of the MAPT locus and (g) in situ hybridization of the MAPT locus of the nucleic acid sample with nucleic acid probes which comprise the H1 haplotype of the MAPT locus; and
wherein the presence of the H1 haplotype of the MAPT locus determines, diagnoses, predicts or identifies the subject as having chronic traumatic encephalopathy and/or prognoses that the subject will have a more rapid clinical decline from chronic traumatic encephalopathy.

33. The method of claim 32, wherein the subject is human.

34. The method of claim 32, wherein the subject is the military or is entering the military.

35. The method of claim 32, wherein the subject plays a sport.

36. The method of claim 35, wherein the sport is selected from the group consisting American football, boxing, ice hockey, wrestling, baseball, cycling, skiing, ski jumping, snowboarding, snowmobiling, bobsledding, luge, ice skating, roller blading, roller skating, inline skating, skateboarding, scooter riding, soccer, basketball, field hockey, softball, water sports, use of powered recreational vehicles, horseback riding, cheerleading, dancing, gymnastics, golf, trampolines, rugby, and lacrosse.

37. The method of claim 32, wherein the subject has suffered one or more traumatic brain injuries.

38. The method of claim 32, wherein the biological tissue is brain or epidermis.

39. The method of claim 32, wherein the bodily fluid is cerebrospinal fluid, saliva, whole blood, buffy coat, serum, plasma, sweat or urine.

40. The method of claim 32, wherein the nucleic acid is RNA, cDNA or genomic DNA.

41. A method of screening, diagnosing, predicting, or identifying chronic traumatic encephalopathy in a subject, comprising:
a. obtaining biological tissue or bodily fluid from the subject;
b. isolating a sample of protein from the biological tissue or bodily fluid;
c. measuring the quantity of Apo E ε4 polypeptide or protein in the sample of protein; and
d. comparing the quantity of Apo E ε4 polypeptide or protein in (c) with a reference value of the quantity of Apo E ε4 polypeptide or protein, the reference value representing a known diagnosis or prediction of normal neurologic function, and finding a deviation in the quantity of the Apo E ε4 polypeptide or protein measured in (c) from the reference value;
wherein if the deviation in quantity of Apo E ε4 polypeptide or protein measured in (c) is increased from or, higher or more than the reference value of the quantity of Apo E ε4 polypeptide or protein, then the subject can be determined, diagnosed, predicted or identified as having chronic traumatic encephalopathy.

42. The method of claim 41, wherein the subject is human.

43. The method of claim 41, wherein the subject is the military or is entering the military.

44. The method of claim 41, wherein the subject plays a sport.

45. The method of claim 44, wherein the subject plays a sport, selected from the group consisting American football, boxing, ice hockey, wrestling, baseball, cycling, skiing, ski jumping, snowboarding, snowmobiling, bobsledding, luge, ice skating, roller blading, roller skating, inline skating, skateboarding, scooter riding, soccer, basketball, field hockey, softball, water sports, use of powered recreational vehicles, horseback riding, cheerleading, dancing, gymnastics, golf, trampolines, rugby, and lacrosse.

46. The method of claim 41, wherein the subject has suffered one or more traumatic brain injuries.

47. The method of claim 41, wherein the biological tissue is brain or epidermis.

48. The method of claim 41, wherein the bodily fluid is cerebrospinal fluid, saliva, whole blood, buffy coat, serum, plasma, sweat or urine.

49. The method of claim 41, wherein the quantity of Apo E ε4 polypeptide or protein in the sample of protein is measured using an antibody that recognizes or binds to Apo E ε4 polypeptide or protein.

50. The method of claim 41, wherein the level of Apo E ε4 polypeptide or protein in the sample of protein is measured by an assay selected from the group consisting of quantitative Western blots, immunoblots, quantitative mass spectrometry, enzyme-linked immunosorbent assays, radioimmunoassays, immunoradiometric assays, immunoenzymatic assays and sandwich assays.

51. A method for screening or identifying an agent for the prevention or treatment of chronic traumatic encephalopathy, comprising contacting or incubating a test agent to a nucleotide comprising the 3′UTR of the tau mRNA from the H1 haplotype of the microtubule-associated protein tau gene and determining if the test agent binds to the nucleotide comprising the 3′UTR of the tau mRNA from the H1 haplotype of the microtubule-associated protein tau gene, wherein if the test agent binds to the nucleotide comprising the 3′UTR of the tau mRNA from the H1 haplotype of the microtubule-associated protein tau gene, the test agent is identified as a therapeutic or preventative agent for chronic traumatic encephalopathy.

52. A method for screening or identifying an agent for the prevention or treatment of chronic traumatic encephalopathy, comprising contacting or incubating a test agent with a nucleotide comprising the 3′UTR of the tau mRNA from the H1 haplotype of the microtubule-associated protein tau gene linked or conjugated to a nucleotide which expresses a measurable phenotype, and measuring the phenotype before and after contact or incubation with the test agent, wherein if the expression of the measurable phenotype is decreased after the contact or incubation with the test agent, the test agent is identified as a therapeutic or preventative agent for chronic traumatic encephalopathy.

53. A method for screening or identifying an agent for the prevention or treatment of chronic traumatic encephalopathy, comprising contacting or incubating a test agent with a host cell or animal possessing a nucleotide comprising the 3′UTR of the tau mRNA from the H1 haplotype of the microtubule-associated protein tau gene linked or conjugated to a nucleotide which expresses a measurable phenotype, and measuring the phenotype before and after contact or incubation with the test agent, wherein if the expression of the measurable phenotype is decreased after the contact or incubation with the test agent, the test agent is identified as a preventative or therapeutic agent for chronic traumatic encephalopathy.

54. A method for screening or identifying an agent for the prevention or treatment of chronic traumatic encephalopathy, comprising contacting or incubating a test agent to a nucleotide comprising the promoter from the H1 haplotype of the microtubule-associated protein tau gene and determining if the test agent binds to the nucleotide comprising the promoter from the H1 haplotype of the microtubule-associated protein tau gene, wherein if the test agent binds to the nucleotide comprising the promoter from the H1 haplotype of the microtubule-associated protein tau gene, the test agent is identified as a therapeutic or preventative agent for chronic traumatic encephalopathy.

55. A method for screening or identifying an agent for the prevention or treatment of chronic traumatic encephalopathy, comprising contacting or incubating a test agent with a nucleotide comprising the promoter from the H1 haplotype of the microtubule-associated protein tau gene linked or conjugated to a nucleotide which expresses a measurable phenotype, and measuring the phenotype before and after contact or incubation with the test agent, wherein if the expression of the measurable phenotype is decreased after the contact or incubation with the test agent, the test agent is identified as a therapeutic or preventative agent for chronic traumatic encephalopathy.

56. A method for screening or identifying an agent for the prevention or treatment of chronic traumatic encephalopathy, comprising contacting or incubating a test agent with a host cell or animal possessing a nucleotide comprising the promoter from the H1 haplotype of the microtubule-associated protein tau gene linked or conjugated to a nucleotide which expresses a measurable phenotype, and measuring the phenotype before and after contact or incubation with the test agent, wherein if the expression of the measurable phenotype is decreased after the contact or incubation with the test agent, the test agent is identified as a preventative or therapeutic agent for chronic traumatic encephalopathy.

57. A method for screening or identifying an agent for the prevention or treatment of chronic traumatic encephalopathy, comprising contacting or incubating a test agent to a nucleotide comprising the Apo E ε4 allele and determining if the test agent binds to the nucleotide comprising the Apo E ε4 allele, wherein if the test agent binds to the nucleotide comprising the Apo E ε4 allele, the test agent is identified as a therapeutic or preventative agent for chronic traumatic encephalopathy.

58. A method for screening or identifying an agent for the prevention or treatment of chronic traumatic encephalopathy, comprising contacting or incubating a test agent with a nucleotide comprising the Apo E ε4 allele linked or conjugated to a nucleotide which expresses a measurable phenotype, and measuring the phenotype before and after contact or incubation with the test agent, wherein if the expression of the measurable phenotype is decreased after the contact or incubation with the test agent, the test agent is identified as a therapeutic or preventative agent for chronic traumatic encephalopathy.

59. A method for screening or identifying an agent for the prevention or treatment of chronic traumatic encephalopathy, comprising contacting or incubating a test agent with a host cell or animal possessing a nucleotide Apo E ε4 allele linked or conjugated to a nucleotide which expresses a measurable phenotype, and measuring the phenotype before and after contact or incubation with the test agent, wherein if the expression of the measurable phenotype is decreased after the contact or incubation with the test agent, the test agent is identified as a preventative or therapeutic agent for chronic traumatic encephalopathy.

60. A method of screening or identifying a test agent for the prevention and/or treatment of chronic traumatic encephalopathy, comprising:
a. contacting the test agent with an Apo E ε4 polypeptide; and
b. detecting the presence of a complex between the test agent and the polypeptide, wherein the presence of the complex between the test agent and the polypeptide would identify the test agent as a therapeutic or preventative agent for chronic traumatic encephalopathy.

61. A method of treating or preventing chronic traumatic encephalopathy comprising administering to a subject in need thereof a therapeutically effective amount of a composition comprising an agent that binds to the 3′UTR of the tau mRNA derived from the microtubule-associated protein tau gene.

62. The method of claim 61, wherein the agent is miRNA.

63. The method of claim 61, wherein the subject is human.

64. The method of claim 61, wherein the composition further comprises a ligand, conjugate, vector, lipid, carrier, adjuvant or diluent.

65. A method of treating or preventing chronic traumatic encephalopathy comprising administering to a subject in need thereof a therapeutically effective amount of a composition comprising an agent that binds to the promoter of the microtubule-associated protein tau gene.

66. The method of claim 65, wherein the subject is human.

67. The method of claim 65, wherein the composition further comprises a ligand, conjugate, vector, lipid, carrier, adjuvant or diluent.

68. A method of treating or preventing chronic traumatic encephalopathy comprising administering to a subject in need thereof a therapeutically effective amount of a composition comprising an agent that binds to the APOE ε4 allele.

69. The method of claim 65, wherein the subject is human.

70. The method of claim 65, wherein the composition further comprises a ligand, conjugate, vector, lipid, carrier, adjuvant or diluent.

71. A method of treating or preventing chronic traumatic encephalopathy comprising administering to a subject in need thereof a therapeutically effective amount of a composition comprising a nucleic acid comprising the H2 haplotype of the microtubule-associated protein tau gene.

72. The method of claim 71, wherein the subject is human.

73. The method of claim 72, wherein the composition further comprises a ligand, conjugate, vector, lipid, carrier, adjuvant or diluent.

74. The method of claim 71, wherein the nucleic acid comprises the sequence comprising SEQ ID NO: 4 or SEQ ID NO: 5.

75. A method of treating or preventing chronic traumatic encephalopathy comprising administering to a subject in need thereof a therapeutically effective amount of a composition comprising a nucleic acid comprising the APOE ε2 or ε3 allele

76. The method of claim 75, wherein the subject is human.

77. The method of claim 75, wherein the composition further comprises a ligand, conjugate, vector, lipid, carrier, adjuvant or diluent.

78. A method of treating or preventing chronic traumatic encephalopathy comprising administering to a subject in need thereof a therapeutically effective amount a composition comprising an agent that blocks or decreases the expression of the Apo E ε4 polypeptide.

79. The method of claim 78, wherein the subject is human.

80. The method of claim 78, wherein the composition further comprises a ligand, conjugate, vector, lipid, carrier, adjuvant or diluent.


SEQUENCE LISTING (8 sequences)

SEQ ID NO: 1 - DNA, 3641 bp, Homo sapiens
agtctgggatccttgagtcctactcagccccagcggaggtgaaggacgtccttccccaggagccggtgagaagcgcagtcgggggcacggggatgagctcaggggcctctagaaagagctgggaccctgggaacccctggcctccaggtagtctcaggagagctactcggggtcgggcttggggagaggaggagcgggggtgaggcaagcagcaggggactggacctgggaagggctgggcagcagagacgacccgacccgctagaaggtggggtggggagagcagctggactgggatgtaagccatagcaggactccacgagttgtcactatcatttatcgagcacctactgggtgtccccagtgtcctcagatctccataactggggagccaggggcagcgacacggtagctagccgtcgattggagaactttaaaatgaggactgaattagctcataaatggaacacggcgcttaactgtgaggttggagcttagaatgtgaagggagaatgaggaatgcgagactgggactgagatggaaccggcggtggggagggggtggggggatggaatttgaaccccgggagaggaagatggaattttctatggaggccgacctggggatggggagataagagaagaccaggagggagttaaatagggaatgggttgggggcggcttggtaaatgtgctgggattaggctgttgcagataatgcaacaaggcttggaaggctaacctggggtgaggccgggttggggccgggctgggggtgggaggagtcctcactggcggttgattgacagtttctccttccccagactggccaatcacaggcaggaagatgaaggttctgtgggctgcgttgctggtcacattcctggcaggtatgggggcggggcttgctcggttccccccgctcctccccctctcatcctcacctcaacctcctggccccattcaggcagaccctgggccccctcttctgaggcttctgtgctgcttcctggctctgaacagcgatttgacgctctctgggcctcggtttcccccatccttgagataggagttagaagttgttttgttgttgttgtttgttgttgttgttttgtttttttgagatgaagtctcgctctgtcgcccaggctggagtgcagtggcgggatctcggctcactgcaagctccgcctcccaggtccacgccattctcctgcctcagcctcccaagtagctgggactacaggcacatgccaccacacccgactaacttttttgtattttcagtagagacggggtttcaccatgttggccaggctggtctggaactcctgacctcaggtgatctgcccgtttcgatctcccaaagtgctgggattacaggcgtgagccaccgcacctggctgggagttagaggtttctaatgcattgcaggcagatagtgaataccagacacggggcagctgtgatctttattctccatcacccccacacagccctgcctggggcacacaaggacactcaatacatgcttttccgctgggcgcggtggctcacccctgtaatcccagcactttgggaggccaaggtgggaggatcacttgagcccaggagttcaacaccagcctgggcaacatagtgagaccctgtctctactaaaaatacaaaaattagccaggcatggtgccacacacctgtgctctcagctactcaggaggctgaggcaggaggatcgcttgagcccagaaggtcaaggttgcagtgaaccatgttcaggccgctgcactccagcctgggtgacagagcaagaccctgtttataaatacataatgctttccaagtgattaaaccgactcccccctcaccctgcccaccatggctccaaagaagcatttgtggagcaccttctgtgtgcccctaggtactagatgcctggacggggtcagaaggaccctgacccaccttgaacttgttccacacaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgaggtgagtgtccccatcctggcccttgaccctcctggtgggcggctatacctccccaggtccaggtttcattctgcccctgtcgctaagtcttggggggcctgggtctctgctggttctagcttcctcttcccatttctgactcctggctttagctctctggaattctctctctcagctttgtctctctctcttcccttctgactcagtctctcacactcgtcctggctctgtctctgtccttccctagctcttttatatagagacagagagatggggtctcactgtgttgcccaggctggtcttgaacttctgggctcaagcgatcctcccgcctcggcctcccaaagtgctgggattagaggcatgagccaccttgcccggcctcctagctccttcttcgtctctgcctctgccctctgcatctgctctctgcatctgtctctgtctccttctctcggcctctgccccgttccttctctccctcttgggtctctctggctcatccccatctcgcccgccccatcccagcccttctccccgcctcccactgtgcgacaccctcccgccctctcggccgcagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgcgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgcatctgctggcctccccctgtgattt

SEQ ID NO: 2 - DNA, 4433 bp, Homo sapiens
accctccctcccttcctcttcttgcagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaataaggtctccattcatggattccaagg

SEQ ID NO: 3 - DNA, 20001 bp, Homo sapiens
atgctacacacaatagttgaaagtgatcatctgaagcttcgcagagggtatctaacccccaatttttgtggatttccccagttatgttttaatttttttttttttttttttttgagacagattctcactctgttgctcaggctggagtgcagtggcatgatcctggctcactgcagcctcgacctcctggactcaggtgatcctcccacttcagcctcctgagtagctgggaccaccatgcccccaagcccagataagtcttgtattttttgcagagacagggttttgccatgttgcacaggctggtctcgaacttctgagctcaagctgtctgccatctcaggctcccaaagtgctgggattacaggcgtgagccaccatgccaatctattttttcatttttaatttaaaacatagactactttttagagcaattttaagttcacagcaaaattgagcagaaagtacagaaaatccccgtataccctctgctctatacagcctcccgcactatcgacatcccccaccagagtggtacatttcttacaatccatgaacctaccttgacatgtcattataacccaaagtccatagtttatattagggctcactcttgctgttgtatactctataggttttgacaaatatacaatcacatgtatccacgattatactagcatacagaacagtctcattgccctaaaaatcccccgtgctctgcctatccatccctccctccctgctaacccctggcagccactgatctttttgctgcctccatagttttgccttttccagaatttcatatcgttgaatcctacagtatgtagccttttcagagtagttatccccaaattcttgaaatcaatataaatatttatttatttacttattttttgagacagagtcttgctgtcatccaggctggagtgcaggcactatctcggctcactccaacctccgcctcccggattcaagcgattctcctgcctcagcctcccaagtagctgggattacaggcatgtaccaccacgcttcgctaatttttgtatttttagaagagacagggtttcaccatgttctcaggctggtctcgaactcctgacctcaggtgatccacccgcctcggcctcccaaagtgctaggattacaggtgtgagccaccatgcctagctagaccaatataaattaataggctgggtgcggtggctcagcttgtaatcccaggactttgggaggccaaggcgggcggctcatgaggtcaggagactgagaccatcctggctaacatggtgaaaccctgtctctactaaaaaatacaaaacattagctgggcgtggtggcaggcacctgtagtcccagctacttgggaggctgaggcaggagaatggcgtgaacccgggaggcggagcttgcagtgagccgagatcgcaccactgcactccagcctggacgacagagcaagactccgtctcaaaaaaccaaccaacaaactatatatatatatatatataactcacctgcaaatcactacattgtaagcaacttaagatcttagtcctcattgtattcacggtgtacagggacactcaaacacctgttgaatggatggaaagatgaatagcctgcctctccgatttcaaaagaaaaaccagaacactaatctagtaacagtgagtggttgtacttagtattacttagaaaaagagatgctaattctttttattccgaataaagaacagcttgtaaaaaatgaagcccaagccaggaacagtggcttacacctgtagtcccagctacttgggagtctgaggcaggaggatggcttgaacccaggagttcaagaccagcctgggcaacacagtgagaacctgtctccacacacacacacaaattatctagacatggtggtgcgcgcttgtagtcccagctactcggatggctgagttgggaggatcgcctgaacccagaagatcgaggctacagtgaggtctgatcacagtattacacttcagcctgggtgacagagtgagaccctgtatctaaataaaataagtatcaatgccagactataggtcatgagatttgactcagatcttaatattttggtaacttttaaatttccgattatatcttccataaagtaggatttcacaactgatagcatggtttctgtgaaagggatttctgctcacccttttcaagttttgtatttggggcactcttcaaatgatctgtcattcccctatgccaggatatccagagtgtctgcacaaagccaaatatgatgagaagatgagctcatggatcggtgatgccttgtccctatttctggaccccaggatcatccttgcttttttgacgatagagtcatgctgctgagtcatgggtaactcaaaccctcatctgcagttaccttcttcttgctagaacccgggaaacctggacaaggggcagttgtcactggcaggataatggagggaagggcaggtgggaaggaggcatccaggaaatagacacacagaaaggaacatgtgggtctaaacaataatcagtgagaggtaagtccagggaaatgaaagcatggatctgcctcacgttatataattaagatgttgaaaatgtccgcataaaccaactcacatttcatttgcaacaggagaactgacattaggggaacctggtaatggttcattttgtagagtgaagtatttgtctgtaatgttaatactctctttctaatttagctgtattttatattgatatttcccaatattggctttgagagagggctgtcgtgtctcatcaaagcaccctgtttagcagacaattaggcagaacaaatgcatgttagtctgtctaaatatgccctgaatttggtgtctgaatttacaatagaaaataaaggaatgttagtcttgaaggctgtcatctgtttcggtagaaaatgcctttttatgaaagtgttttcatgaaactatttcaaagagtaagctgctcatgataactgggaagaattaccaagtatgatggcctcttagcattattgtggctgttccccaatgtccccttctcctcctatagctggtatttagctggacacaagtccctgtaataaaaatatattttccagcctcgatggcaactttgtgtggtaatgtctaaattctggccaataggatgttagcaaggatgtcatatgtgacttccaggaaatgtccttaaagtaaagttgttctcttcctcatctcttccttctttctgggggctagaatgcagatgtaatgactggagctcaggcagctgttttggactatgaggcagcatgctaaggatggtagagaaatcatacagctggataagagctccctgatggtcttggagcttctaaaacagcctttgactcctacttctggacttctatgtaagagagaaataagagaaattgctgtcccttagcaatcatggttgggcaagggagtagcaaccggtgcccaggtaacaaacgtgttcctccctgctgcactgtataattgcagccctacttcctgctaatgactaaagttaattatcccttccaagatggtaacttttctttttgcctgctgatgtctggacctaaggaagcagaagggctcaggcagcagccacagctgatagttcaataaaactgtgctgtgtcccctggtggaagttttccctttgggagaccaggacctctaaccctgaagagcttagagctgtaatgatgtggcggtaaatgcggccacttcagttttcacccttggttcccaaacccatgtattttttcctgtcggggtttgttttctatcgctgctggaaccaattaaagactgggatacgttctaagaaatgcttcattaggtgatttcttcatgtgagcgtcatagagtgcacttacacagcctagatggcactaggtactactaaatgcctgggctatatggtatagccttttgctcctaggctacaaacctgtacagcatgttactgtactgagtactgcagcaacggtaacacaatagtaagtatttgtgtatctaaatgtgtctaaacatagtaaatgtatagtaaaagcagtataaaatagtataaacgcagcggctcatgccagtaatcccagcactttgggaggcccagccaggaagatcacttgagcccaggagttcgagatcagcctaggcaacacagcgagaccttgtctctacaaaaaatcaaaaattagccaggtatggtggcatgcacctgcaatcccagctacccaggaggctgaggtaggaggattgcttgagcccaggagttcaaggttgcagtgagccatgattgcaccactgcactctaccctggcagacagagtgagactctatctcaaaaaataaaataaaataaaataaaataaaaagttgaaaaacagtacctctgtttaggacacttaccatgaatggaacttgtaggactggaagttgctctgggtgagtcagtgagtgagtggtgagtgaatgtaaaggcctaggacatcactgtacactactgttgacttttttttttttttttgagatggaatttcactcttcttgcccaggctggagtgcaatggcaggagcttggctcaccgcaacctctgcctcccgggttcaagagattctcctacctcagcctcccaagtagctgggattacaggcatgcaccaccacacccagataattttttgtatttttagtagagattgggtttctcaatggtggtcaggctggtcttgaactcctgacctcaggtgacccacccgcctcagcctcccaaagtgctgggattacaggtgtgagccacagctcccagcctgctatcaactttataaacactgtacatttagtccatattaaagttattaaaattatttttcttcaacaataaattaaacttagcacactgtaacttgttttgctttataaactcttaaatttttaaacctttttgacttttgtagtaacgtttagcttaaaacacaaatacattttcagctgtacaaaaattctttctttatgtccttatcctataagcttttttctattttgtttttttttacttgttacacttttcttggtaaaaactaagacatcaacacacatattagcctagtcctacaaagggtcaaggtcatcagtatcaccaccatcttctacctccccatcttgtcccactggaaggtcttcaggggcagtaacatgcatgaagccgtcatctcctattataacaatgccttcttctggaatccctcacgaaggccctgcctgaggctgtttcacagttaacttttttttttaataagtagaagaagtacactctagaataataataaaaaatatagtatagtaagtacccaaaccagtaacacagtcgtttattatcattatccaattttttttttcaaatggagtctcactctgttgcccaggctggagtgcaatggcgcgatctccactcactgcaacctccacctcccatgttcaagtgattctcctgcctcagcctcctgagtagctgcaattgcaggtgcccgctaccacgcccggctaatttttgtatttttagtagaaacagggtttcaccatgttggccagggtggtctcaaactcctgacctcaggtgatccacccaccttggcctcccagagtgctgggattacaggtatgagccattgcacccagccatcattatccaatattatgtactgtacataattgtatgtgctagacttttatacaacagccagcaagtaggtttgcataccggcatcatcacaaacacaggagtaatgtattgtgctacaacatcatcaccgctatgaagtcactaggcagtagggatttttcagctccagtgtaatcttatgggactgccgtcataaatgcagtccattgtggttgtctggtgtatggctgtgctacaaactcagtggcttaaaacaatgcaaatatatcatcttagagttctgtaggtcagaagtccaacaaagacctcactaggctaaaatcaaggtgtcgacagggaggctctagcagaggatcctgcatcctttgtaaagaatttttacccttgtaaagaattgatggttcggctgtgccttccaagctgtgcttgaccaattctctcagggtagccgcttctagatggagaagcctatgatataaccagtggatcccgtggctgtgggttcactcccacccctcccctgcttcacctcttccagtttctagagtaggtggactccttagcacttggcctcttcctcatcttcaaagccagcaacatcacggccgggtgctgtggctcatgcctgtaatcccagcactttgggagactgaggctggtggatcacttgaggccaggagtttcagaccagcctgaccaacatggcaaaaccctgtctctactaaaagtacaaaaaaaaataaaataaaattagctgggcgtggtggcacacgcctgtgatctcagctactcgggaggctgaggcaggagaatcgcttgaacccaggagatggaggttgcagtgagctgagatgtcgctactgcactccagcctgggtgacagagtgagaccctgtctcaaaaaaaaaaaaaaaaaaaaaagccagcaacgttacatctctctgatccttcttctgtgtcacatctctcactcttactgcagccaggaaattatttccacttttaaggactcttgtgactatactgggctcacctggataatcggggatccacccgtcatctcaaggtccctacttttcatcacatttgcaaagttccttttgctatgtacactaacacattcacaggtcccacatggtgaaaccccgtttctactaaaaacacaaaaattagtcaggtgtggtggtgtgcacctgcagtcccagctactcgggaggctgaggcaggagaattccttgagcccaggaggcggaggctgcggtgagccaagattgcgccactgcactccagcctgggcaacagagcgagaccccatctcaaaacaaacaaacaaacacacaaacacaaacaaacaaacaaaaaccacattcacaggtcccagggacttgggcatgaacttctttagggccatttttctaccacaaggctgtaaaaaggtctttgattcaaggtctgtcctgcatcctggagtacgtctcctacccttgtaaagaattgatggttcaggtgtgccttccaaactcagggcagctgcttctagatggagaagcctatgaattaactagaggatcccgtggctgtgggttcagtcccacccctcccctgctgtaaagcagttcccttggtctcatgtgacactatgtgggaccctgtactggagaatcaaacacactgtaagccctcagataaggatactggcttaggcaccgaaggcaggaaaggcaaatccgtactcagatcatgggtctactcctgtcagaacaaattactgggccttatgtgtggagggaccccaacaaagtcaacctgccggttcgtctttttgagaaatggtccattctgaagacttagcactggtctctgttgcgggaggttggacatttggtggcagcggtagccttggtacatgggacccatcctgcacatagcatccatttctgtcaccactgctgcttcattcgtatgtccatcaggtcagcattgggacggcccatggcagaggccaactggcatcccccactctcgtcctcttgtctacttggttgtccactgtctcttccatagtaggtcatctctagtgggcattaacacgtggcacaaagatcaagctttgtgtgctcttttttttttttttttttttctggagacagagttgctctttcgcccaggctggagtgcagtgacgcgatcttggctcactgcaacctctgcctcccaggttcaaaccattgtcctgcctcagcctcccaagtagctgggattataggcacgggccaccacacccagctaatttttgtatttttactagatacgaggtttcaccatgttggccaggctgttctcgaactcctgacctcaggtgatccacctgcctcggcctcccaaagtgctgggattacaggtaccagccaccaggcctggccctttgtgtactcttgcctttgtcatccacatgcctctgcccagatctccttgtctccagtcttctaattgttccctttccacatctatgatcagctggccaggccattcaccactacccaaaagtttgtatatattctcaccttgagctctgctctgtccatacaaaatgggtgaacagctacaccacccaaatccctgcccattaggagggttttgccttgccactctctttcaaggccacccttgagtgagattgtagtacatttcctgtcgttttcagcctaaacccacaaaccaagccaagccattcacaaaccaaacttgctctttcctcttccatcagctggtcagaaggacccctcctataaccttaggtgtgtgtagagggagggtgctggtgcaactccggtgggtggcttgggagtctgaacagactgcacgtgcagcttgcttgtggcccaggtcctgtccatgctcaatcccagatgtacaacttccatctaacaatggattgttgctgggcttgcctaaccttataatttaactcatctgatgtaacccagttaatgaagggcagttccaggtgcaagatcagttggtatcccaccatcaggtgctccatttctaccagggcacagcagcgtgccaggagttgttttgccaaaggcatatgatcctccactgcagatggcatggccttgctccagaaccctgggagcctgcactgtgactctcccactggggcatgctacaagctctacacagcatcttcttcatcatcatgccttttgtactatagagtctgctgggttgatggctcaatcgcaaggctacttgcactgctgcctggacctactgcagagccctttcctgttctgggccccaattcaagctgacagcctttcaggttaactggttattattggtcaagtggtattcccaggtgtggaatatgctatctccagaagccaaagagaatgtctaagttttgtgcttccttcttcatggtgagaaatatgagatgcaatcatttgattttgactttggatggactgtcctgacatgctcctgtccacaggacccctaaaattgttcctggtgtgcaggctcctaaatatctgtagtattcatctcccaccctctagagtgtgtgtaacctaccaatgcctttaacatgctagccactttttgctcctttggcccatttagcaggatgtcattcacataacagatcaaggccatgttctgcaaaatgtccagatggcccagatctctttgtatcattcagggtccaaaacagggacagaagccacagagtaatttgaacaggaaaagtttaagataaagaagcagtgattccaacagaggaggaattataagggtgctggtgagcagtgcacagaggtagtcggggcatcaggagcctgcttgcactgaggcagagtaaggactgaggtgtgccatatctgtattaggggggccccaggaagcaaccctgcattatacaggtgagccagtgctggaaggtggatgcacaggaggagtcggtgtgtcaggagcttgatccctagcagagcagtgtaaatcctgggtgcatgtaacctcattgggagagccacagtgagctgggcacctagctggggccaatgctgtgagctcaccaaggaccccacactctgcgtatggagctggggcagaggccctctctcctgtgcctctggcaaccatacagtagaggcaagaagaagcaaaacccaagcatgacagaaacagcaagaaaagccttttcttttcttttctttttcttttttcttttttctttttttttgagatggagtctcactctattgcccaggctggagtgcagtggtgtgatcttggctcactgcaagctccacctcctggattcaagtgattctcctgcctcagcctcctgagtagctgggattacaggtgcatgccaccatacccagctaatttttgtatttttagtagagacaaggtttcaccatgttggccaggcaggtcttgaactcctgacctcaggtgatccacctgcctcagcctcccaaagtgctaggattacaggcatgagccaccacacccagctggaaaggcttttctttctgctttgtcgctctagggcatctacaacaaagcttaacctgatgctcaccacaagggagaaatgcttaaagtgtctagctccattatcacaaagcaggtaatgcagggtgacttggagatgagagtcaatacattgaaaaactggcacagaccagactgtgacagacggtgggaaagttaatgtatgggggggagttgtaaatgtatactgttgtccattccatgtgaatgtggactattttgaatcctcttttcagataggaagagaaaagaatgaattctctaacaatggccacacactatgtgtctgaaactgttcatctattctagcaaagatctggcatggtaggtgcaatggcagctactactccatttactttgcagtagtctagtgtcattccatagaatccctcttctcctacatgaaccatactagagacatgaaggtaactattacccttgcatccttcagttccccaagggtggcattaaccttctcaactctttcatcccgggatgcaatattgtttctgatttattatgttgacctgggttggaaggggactgcttggaggcttctcaactactatagctcttactccacaagccagctcagtgtgtatataggagaggatcacacacactccaagaattcatccttggtttcttttgatggtgcaggttgagcagtcccgttttcagtctcccatctctctttgccctcagggactctgtgctctattacccacttccacaactctctgtagatcaggctcctgggctgtcactccacctgtagatcacatccgggtcaaatatttctatctcgatttttcatgtgggagataatgactggatggggtccgtggacccagtggactcactgtcagccagaccttggccgggactcaatttattaccaggtctccaaatacttcctctctaacgaaggccgtgatgatacttcaggcctctggttatcaatgccagctcagacactgtgtcttacagtcctcaggatatttgggtattcctcttttcccatttgccatggtgctgcacagtccttcttaatgaggagccaggcttcttctcaatcaattggttctggatctgaaaactacctcaggtctgaaaactgggcaacgtgtgactttttactggggtgtctgccttagcttcctgatcattcatcatgatttattctaatggtgcaggttgagcagtacccttttgggtctcccatctctttttcttttctttgtttgtttttgtttttttgtgagacagagtcttgctctgtcacccaggctggattgcagtggcacaaggtcggctcactgcaacctccgcctcccaggttcaaatgattctcctgcctcagcctcctgagtagctggcccttcaggtgcccgccaccacgcccggctaatttttgtatttttggtagagatggggtttcaccatattgactaggctggtctcaaactcctgaccttgtgatctgcccacctcagcctcccaaagtgctggtattacaggcatgagccacctcgcctggcctggtctcccatctcttttgcccccatggactctgtgctctatcacccacttccataactctctgtagaccaggctcctgggctgccactccaacctggccagttactacaatcagtgcacttgcctggcttctgatggtgccccagttgtgcctcctcaggtagtggaaggaatcacaagaagactcctgaaagtatactcacatagggctttcataaaactaaagaaaataatatagcaggataaaggcaaacaccagggagctgtccgacatccaggcgcatcttcctttcaggtacataggatgtacttgatcttaggatgataaaccaccaagatacatgcaaaatgccttggtctcagggaagtcacaatctcatctaagatggtttttaatatccttctggtcacatagccaaaaccagcttgcatgatcaggctcaactaagataatctaacagaaaccaggtgaaaataatcaatctgtacattttcacttaacaatggtgacaagttggtacagaatatctgtagtagtttttggactcagtacagaactacactaatcaatggtttgcaaattccattctggatggatcacgactcagcactatggcttcgggatcccctagagataagcacaaggttgcagccaggaaaaatgacccccatacccacacagatagttaagggccaaaccacctctgttgttttgggtcctatcattctctctgctatcagtaggccttattctataagaacttcttcaatccagtattggcctacagagacagctaagtctacattttatttatttatttatttatttatttatttatttatttacttatgacacaggttcttgctctttcccccaggctggagtgcagtaggtgtgatcttggctcattgcagccttgaacttccaggccctgcctcagcctcccaagtggctgggactacaggtgtgcgccacaaagcctggttaatttttgcatttttttgcagaaacggggttttgccacattgcccaggttggtctctcactcctgggctctagtgatccttctgccttggcctcccaaagtattgagattacaggtgtgagccaccctacccggcctgaattttttattgatgctggtgtcccctctcatcagtacactctgatgtcctcagaatggttgtcctctgcgcctccctatggaacattaccatcaggtgggttttctggactttttgcagtgtgtctactccagaaggttactgcccttccccttgtgacaccttcctctactgtccaccaggacaatgatggcatttctaccccatttactacgggctttattttttatttttatttatttatttatttattttttgagacaacagtctcactctgttgtccaggctgtagtgcagtggcacgatctcagctcactgcaacctccgcctcccaggttcaagcagtctttctgcctcagtccaccagtagctgggattacaggcatttgccaccatgcccggcttgtttttgtattttttttttttagtagagacagggtttcaccacgttggccaggctggtctcaaactcctgacttcaggtgatccacctgcctcagcctcccaaagtgctgggattacagatgtgagccactgcacccagcaggcctttatttttttcatgcttctaagagtgagttcatagaatctctgggagcctttgctaggatgttaaattgtgtattatgggatagaatgctcccaagtcaacacagtctccctcatccaatctcatgttctggccaccttgatcaagcaccctcagcattcagtcccacatgtccttcccagcttcttgctggtgtatgctggttaggtcttgcagctactttggggtataatcccttttctcctttattaggcccatcatgtccctgagttatactgtgacttaaccctagtttacatttcatggccaggaggggaggtgggttccttgttgactgtgggaaaaagccttggcattttcgagtaggagccagggaagggcacttctgcaggctcagagcattcaagagaatctggaaatccaagattttcaggggcatcaactcagatgtccctgtttcacatgtcagggtcccatcctttcccaacaagggctctgaccttgatatagcaggcctaccttgcttggaagctgcattcgtcctttctcatgctgctaataaagacataccaaagattgggtaatttataaaggaaaaaggtttaattgactcacagttcagcatgtctggggaagtgttaggaaacttacaatcatggcagaagagaaagcaaaccatcctttctcacatggtgacaggaagagcaaagcggggtaagccccttacaaaaccaccagatctcatgagaactcactatcacgagaacaccatggaggtaactgcccccatgattcaattacctcccaccaggtccctcccacgacatgtggggattatgcgaactccaactcaagatgagatttgggtggggacacagccaaaccatatcagaagcttaaccttctttggagcatgattattcagttgaacctaagttcagtagtcacccagttatgctgtcttcagctactattttccatatgtttctcaaacatctgatatatcacactggctagtgcactttcttccaccagcataccatctcaatttaccactttaacaattggactgccactttgtgtcagggactatctgtgctccaactactacaagtgataaggtcctcactgacagccagggagcaagtgatccagctctaaaactcaccttatcatctgctttcctagaccactcctaacaaccaactattctgggttgagttctccaagaggcagagagttcaggatacagaatgttgttttgtttttgttgttgttgctgttgttgtttgtgtgtgtgtttgggcttttttgagacggagtctcactctgttgcccaggtagaagtgcagtggcatgatctcagctccctgcaacctccacctcctgggtttaagtgattcccctgcctccacctcctgagtagctgggactacaagtgtgcgccaccacacccagctaatttttgtgtttttagtagaaatggggttttaccatgttggctaggctgctcccaaactcctgacctccagtgatccacctacctctgcctcccaaagtgctgggattacaggcgtgagccaccacacccagcccagaatgtttattagaatgcacaattaataccagaggcagtggggaaggaaggactgagcagaggaggaagttgagttgtgattcaacccaacaactgcctggctggcatggggagctctggagttaaatagggccatcagactttcccagtgtggggccaacatgactgggtctttatacccccacctctgtcagtcactcaacgtggtctccctgcaacaaggtgactcttgcagccgagacaatccctgaagggacagagggctgaagcctgtctgccaacagcactcccagtggctggaacaagtccttccctataggggaatctgggcggcacacctccatctccatgtccatcacatacgatatcacagacatttaaatattttgataactgtacataagagtttcctttataatcttatagatcttattttatgcatttgaaaatattcttctgagacagggcttttatcatattgccatagggtgccacgatataaaaaaggttaaatactctctgattcagaagtatccaatgatgacttctctctcatgcatttaattgaaaatctggtttttctccttctctgctagttctctacctctctccccacctcccacatcatagcctattcacatatgtctgaatctcatgatagacaagttcaggttcttttcccaggttctttttaccacatccccccacccccacataaaaagtatatatggcacagcctaggttccacccaaatcctttctcctcttcttcctgggcccacaactctcctacatacattggtataccttgcgcttagggatggccatgtgactaagttctaacagtggaacatgatcagatgccacttccagcctctaagacagccagtgtgtttcctccataagctccttctcttcctcccaactggagactctaaatgatgaccctgcctcaagcaagcaaacaacaagtccctcaggggtggtgtaggctgcaaatggaaggagcttgagtcccaaaccttccacggagaaggctggctaccaacctggatcactcacccaagactgctcgaagagttggtttgaaccattgtgttttggggtctatttattacaacagtttagcttgctttgtgaatagatttagtggcagagcctccaaattctatagatacattgatctcagtcctaaccgcatctggaacaccattaaataaaggaattgcaaacccagagaaggtaatgaatttgtctaaggtcatacaagatggctaggatcaggacccaactctccagttttctttcttctctgctattctgccttctgtgatcctacataagtgggcatgattgtataacatatgcggccatgagatttctctttcagcaagagaaagggacaggaagaaagagagggaatgcattttcttggcctgaattagtgtgagccattagttacctacattgactaaattatctggaatgaacattcaactctacatcacatatagttaaaatgacagatctgcttaagattgtttctagcatacgttatttcaatttaggcaaatgtgaccattcagtgtgaggggaccatactgtcattaggtccctgtcagttctcaattatactgttatcttagagggggaaaaatgtgaaatttgaatgtagacgagtgttgatttgactgctacagtttattttacgtatagaaataaaataatgtgtagcaaaagcattattacaaagatgataatgaaataactagtatttataatagtataatagtatagtatttataatagtatgatagtttaatgactatttgtcagatgttgtgtaagaaactttatacacacacacacacacacctcatttaattcctgtatcaatcaggatacaggacgctgtggtaacaactcctcaaatctcggtggcttgcacaacaaatgcttatttcttttttttttttgacaccaagtcttgctctgtaacaggctggagtgcaatggtgcaatctcggctcactgcagcctctgcctcctgggttcaagcgattctcctgcctcagtctctcgagtagctgggaacacaggcacgcgccaccacatctggctaatttttgtgattttagtagagatgggatttcaccatgttgctcaggctggccttgaactcctgacctcaagcgatccacccacctcagcctcccaaagtgctgggattacaggcatgagccactgcgcccagccccaaatgtttatttcttgctcatgtgacatgtacttcctcgagtttttccttcctgagatctaagctgaaggaacagctctctggagccacgccattctggtggcggaaaggaagagtaaaagtggtagaaccttgcaatgctcttgaagcgcctatttggaatgtctacatcatgtaaatggtaatggacaagtatgtataatccccacaccaaaaaaaggggacactattggggacaataaccacatttcaatgctgcaagacggatattgactgcacccccttcccactttcagaaagaagaagagtaattttgctgaactccttctagagactggaaatgtcccttccagttggggtgattagggaaggctttggtaaaatttgagctagagtttgaaggttaggtagactactggtgggtgaagaaagaacaaggacctttgtaggcaaaggaaaacctcagaattacagaggtggaaaaagagttctagtcaagccacttcagctggctacagagtaggtgggaaagaaaatgggaggacaagggctcagatgatggggggttggggcattggggggacacttgaaagctaaactaaggggttgaacttaatttaggaggcagttagaagcttttacatatttttgagcaagagagtgacataattaaaatgatctgggccaggtgtggtggctcacacctgtaatcccagcactttgggaggctgaggagcttgggtcacctgaggtcaggagatcgagaccagcctggccaacatggtgaaatcccgtcctactaaaaatacaaaaattagccgggagtggtggcatatgcctgtaatcccagtagctgggaggctgagacaggaaaatcgcttgaacccgggaaacaggttgcagtgagccgagatcgtgccactgcactccagcctgggcaacagagcgagactccatctcaaaaaaacaaaacaaacacacacaaaaaaccaaaaataaataaataaaatgatcacttctgaatactgatctaactaggggttgcagggtgggctgatatagggagaaactggagagcaaggagatcactaaggtccctacatgtccagaaccaagatagaggtcttgaactaggatggtggcagttagaacaacaacaacaaaaagtcaattccaggctgagtgcagtggctcatgcctgtaatcccaacgctttgggaggctgaggtgggagttagaaagcagcctgggcaacactgcaagacctcctctctaaaaaaaaaaaaaaaaaaaagttagccaggtgtggtggtgcccacctgtagtcccagcaactcagaaggctgaggtgggaagattgcttgagccccaggagttcaagcttgccgtgagctacgattgtgccactgcactccagcctgagcaagaccttgtctccaaaaaaaggtcaattccactgacttttctaaggtgtacaccatcaaggggcagctccatctccaggccattggctcatgagacattctgtagtcagaaggctagggcagattgctttgagcaagcccccatggtggttctcactcctacttctttgggtatatgcccctctgtttaaaaataaagttaatatgcatttaaaaaaaaaaaggagaaaaaggtcagttccagaaactgtgtgaataaagcattttacttgctttttctattaatctataacatatgttgattttttaaaaagaatataagagctatgcaaattggagcttcaagacaacttcccatctccctaggaggagatggctgccctaaacccccctacatagaaatcatcccactgcttgggcttaaacttgatgttggggaaatgaaaaatccaagctaaggccgaagcctggggcctgggcgaccagcagaatgaggaccactggtcagtttcaggctgaggtgcgtcttccaggggacaatctctagctggcccttaaacattcagacttcaagctctatttacagcataaaggtgtttcaaaagacgtgatacaaataactgcaaatgctctgcgatgtgttaagcactgtttgaaattcgtctaatttaagatttttttttctgacgtaacggttagattcacgtttctttttttttaagtacagttctactgtattgtaactgagttagcttgctttaagccgatttgttaaggaaaggattcaccttggtcagtaacaaaaaaggtgggaaaaaagcaaggagaaaggaagcagcctgggggaaagagaccttagccaggggggcggtttcgggactacgaagggtcggggcggacggactcgagggccggccacgtggaaggccgctcaggacttctgtaggagaggacaccgccccaggctgactgaaagtaaagggcagcggacccagcggcggagccactggccttgccccgaccccgcatggcccgaaggaggacacccacccccgcaacgacacaaagactccaactacaggaggtggagaaagcgcgtgcgccacggaacgcgcgtgcgcgctgcggtcagcgccgcggcctgaggcgtagcgggagggggaccgcgaaagggcagcgccgagaggaacgagccgggagacgcc

SEQ ID NO: 4 - DNA, 4503 bp, Homo sapiens
ctggcacttcatctcaccctccctcccttcctcttcttgcagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataatcgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatattttaaaaaaaaacattcaaaaacatggccacatccaacatttcctcaggcaattccttttgattcttttttcttcccccctccatgtagaagagggggaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccaccggcatctctggagtgtgtgggggtctgggaggcgggtcccgagccccctgtccttcccacggccactgcagtcacccctgtctgccccgctgtgctgttgtctgccgtgagagcccaatcactgcctatacccctcatcacgtcacaatgtcccgaattcccagcctcaccaccccttctcagtaatgaccctggttggttgcaggaggtacctactccatactgagggtgaaattaagggaaggcaaagtccaggcaccagagtgggaccccagcctctcactctcagttccactcatccaactgggaccctcaccacgaatctcacgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttctttccccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcatctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcacagcagtcccaaaggaggcttacaactcctgcatcacaagaaaaaggaagccactgccagctggggggatctgcagctcccagaagctccgtgagcctcagcctacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttgcagtagaaatccagggcctcccctggggctggcagcttcgtgtgcagctagagctttacctgcaaggaagtctctgggcccagaactctccaccaagagcctccctgccgttcgctgagtcccagcaattctaagttgaagggatctgagaaggagaaggaaatgtggggtagatttggtggtggttagagatatgcccccctcattactgccaacagtttcggccgcatttcttcacgcacctcggttcctcttcctgaagttcttgtgccctgctcttcagcaccatgggccttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcggcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaataaggtctccattcatggattccaaggacaagaaagtcatatagaatgtctattttttaagttctttcccacgcacccttagat

SEQ ID NO: 5 - DNA, 20002 bp, Homo sapiens
cggcgtctcccggctcgttcctctcggcgctgccctttcgcggtccccctcccgctacgcctcaggccgcggcgctgaccgcagcgcgcacgcgcgttccgtggcgcacgcgctttctccacctcctgtagttggagtctttgtctcgttgcgggggtgggtgtcctccttcgggccatgcggggtcggggcaaggccagtggctccgccgctgggtccgctgccctttactttcagtcagcctggggcggtgtcctctcctacagaggtcctgagcggccttccacgtgggcggccctcgagtccgtccgccccgacccttcgtagtcccgaaaccgcccccctggctaaggtctctttcccccaggctgcttcctttctccttgcttttttcccaccttttttgttactgaccaaggtgaatcctttccttaacaaatcggcttaaagcaagctaactcagttacaatacagtagaactgtacttaaaaaaaaagaaacgtgaatctatgatgtatctaaccgttacgtcagaaaaaaaaatcttagacgaatttcaaacagtgcttaacacatcgcagagcatttgcagttatttgtatcacgtcttttgaaacacctttatgctgtaaatagagcttgaagtctgaatgtttaagggccagctagagattgtcccctggaagacgcacctcagcctgaaactgaccagtggtcctcattctgctggtcgcccaggccccaggcttcggccttagcttggatttttcatttccccaacatcaagtttaagcccaagcagtgggatgatttctatgtaggggggtttagggcagccatctcctcctagggagatgggaagttgtcttgaagctccaatttgcatagctcttatattctttttaaaaaatcaacatatgttatagattaatagaaaaagcaagtaaaatgctttattcacacagtttctggaactgacctttttctcctttttttttttaaatgcatattaactttatttttaaacagaggggcatatacccaaagaagtaggagtgagaaccaccatgggggcttgctcaaagcaatctgacctagccttctgactacagaatgtctcatgagccaatggcctggagatggagctgccccttgatggtgtacaccttagaaaagtcagtggaattgacctttttttggagacaaggtcttgctcaggctggagtgcagtggcacaatcgtagctcacggcaagcttgaactcctggggctcaagcaatcttcccacctcagccttctgagttgctgggactacaggtgggcaccaccacacctggctaacttttttttttttttttttttttttagagaggaggtcttgcagtgttgcccaggctgctttctaactcccacctcagcctcccaaagcgttgggattacaggcatgagccactgtactcagcctggaattgactttttgttgttgttgttctaactgccaccatcctagttcaagacctctatcttggttctggacatgtagggaccttagtgatctccttgctctccagtttctccctatatcagcccaccctgcaacccctggttagatcagtattcagaagtgatcattttatttatttatttttgtttttttgtgtgtgtttgttttgtttttttgagatggagtctcgctctgttgcccaggctggagtgcagtggcacgatctcggctcactgcaacctctgtttcccgggttcaagcaattttcctgtctcagcctcccagctactgggattacaggcatgtgccaccactcccggctaatttttgtatttttagtaggacgggatttcactatgttggccaggctggtctcgatctcctgacctcaggtgacccaagctcctcagcctcccaaagtgccgggattacaggtgtgagccaccacacctggcccagatcattttaattatgtcaatctcttgctcaaaaatatgtaaaagcttctaactgcctcctaaattaagttcaaccccttagtttagctttcaagtgtccccccaatgcccctaccccccatcatctgagcccttgtcctcccattttctttcccacctactctgtagccagctgaagtggcttgactagaactctttttccacctctgtaattctgaggttttcctttgcctacaaaggtccttgttctttcttcacccaccagtagtctacctaaccttcaaactctagctcaaattttaccaaagccttccctaatcaccccaactggaagggacatttccagtctctagaaggagttcagcaaaattactcttcttctttctgaaagtgggaagggggtgcagtcaatatccgtcttgcagcattgaaatgtggttattgtccccaatagtgtcccctttttttggtgtggggattatacatacttgtccattaccatttacatgatgtagacattccaaataggcgcttcaagaggcattgcaaggttctaccacttttactcttcctttccgccaccagaatggcgtggctccagagagctgttccttcagcttagatctcaggatggaaaaactcgaggaagtacatgtcacatgaacaagaaataaacatttggggctgggcgcagtggctcatgcctgtaatcccagcactttgggaggctgaggtgggtggatcgcttgaggtcaggagttcaaggccagcctgagcaacgtggtgaaatcccatctctactaaagtcacaaaaattagccagatgtggtggcgcgtgcctgtgttcccagctactcgagaggctgaggcaggagaatcgcttgaacccaggaggcagaggctgcagtgagccgagattgcaccactgcactccagcctgttacagagcaagacttggtgtcaaaaaaaaaaaaaaagaaataagcatttgttgtgcaagccaccgagatttgaggagttgttaccacagcgtcctgtatcctgattgatacaggaattaaatgaggtgtgtgtgtgtgtgtgtgtgtgtgtataaagtttcttacacaacatctgacaaatagtcattaaactatcatactattataaatactatactattatactattataaatactagttatttcattatcatctttgtaataatgcttttgctacacattattttatttctatatgtaaaataaactgtagcagtcaaatcaacactcgtctacattcaaatttcacatttttccccctctaagataacagtataattgagaactgacagggacctaatgacactatggtcccctcacactgaatggtcacatttgcctaaattgaaataatgtatgctagaaacaatcttaagcagatctgtcattttaactatatgtgatgtagagttgaatgttcattccagataatttagtcaatgtaggtaactaatggctcacactaattcaggccaagaaaatgcattccctctctttcttcctgtccctttctcttgctgaaagagaaatctcatggccgcatatgttacacaatcatgcccacttatgtaggatcacagaaggcagaatagcagagaagaaagaaaactggagagttgggtcctgatcctagccatcttgtatgaccttagacaaattcattaccttctctgggtttgcaattcctttatttaatggtgttccagatgcggttaggactgagatcaatgtatctatagaatttggaggctctgccactaaatctattcacaaagcaagctaaactgttgtaataaatagaccccaaaacacaatggttcaaaccaactcttcgagcagtcttgggtgagtgatccaggttggtagccagccttctccgtggaaggtttgggactcaagctccttccatttgcagcctacaccacccctgagggacttgttgtttgcttgcttgaggcagggtcatcatttagagtctccagttgggaggaagagaaggagcttatggaggaaacacactggctgtcttagaggctggaagtggcatctgatcatgttccactgttagaacttagtcacatggccatccctaagcgcaaggtataccaatgtatgtaggagagttgtgggcccaggaagaagaggagaaaggatttgggtggaacctaggctgtgccatatatactttttatgtgggggtgggggggtgtggtaaaaagaacctgggaaaagaacctgaacttgtctatcatgagattcagacatatgtgaataggctatgatgtgggaggtggggagagaggtagagaactagcagagaaggagaaaaaccagattttcaattaaatgcatgagagagaagtcatcattggatacttctgaatcagagagtatttaaccttttttatatcgtggcaccctatggcaatatgataaaagccctgtctcagaagaatattttcaaatgcataaaataagatctataagattataaaggaaactcttatgtacagttatcaaaatatttaaatgtctgtgatatcgtatgtgatggacatggagatggaggtgtgccgcccagattcccctatagggaaggacttgttccagccactgggagtgctgttggccgacaggcttcagccctctgtcccttcagggattgtctcggctgcaagagtcaccttgttgcagggagaccacgttgagtgactgacagaggtgggggtataaagacccagtcatgttggccccacactgggaaagtctgatggccctatttaactccagagctccccatgccagccaggcagctgttgggttgaatcacaactcaacttcctcctctgctcagtcctccttccttccccactgcctctggtattaattgtgcattctaataaacattctgggctgggtgtggtggctcacgcctgtaatcccagcactttgggaggcagaggtaggtggatcacttgaggtcaggagtttgggagcagcctagccaacatggtaaaaccccgtttctactaaaaacacaaaaattagctgggtgtggtggcgcacacttgtagtcccagctactcaggaggtggaggcaggggaatcacttaaacccaggaggtggaggttgcagggagctgagatcatgccactgcacttcaacctgggcaacagagtgagactccgtctcaaaaaagcccaaacacacacacaaacaacaacagcaacaacaacaaaaacaaaacaacattctgtatcctgaactctctgcctcttggagaactcaacccagaatagttggttgttaggagtggtctaggaaagcagatgataaggtgagttttagagctggatcacttgctccctggctgtcagtgaggaccttatcacttgtagtagttggagcacagatagtccctgacacaaagtggcagtccaattgttaaagtggtaaattgagatggtatgctggtggaagaaagtgcaatagccagtgtgatatatcagacgtttgagaaacatatggaaaatagtagctgaagacagcataactgggtgactactgaacttaggttcaactgaataatcattctccaaagaaggttaagcttctgatatggtttggctgtgtccccacccaaatctcatcttgagttggagttcccataatccccacatgtcgtgggagggacctggtgggaggtaattgaatcatgggggcagttacctccatggtgttctcgtgatagtgagttctcatgagatctggtggttttgtaaggggcttaccccgctttgctcttcctgtcaccacgtgagaaaggatggtttgctttctcttctgccatgattgtaagtttcctaacacttccccagacatgctgaactgtgagtcaattaaacctttttcctttataaattacccaatctttggtatgtctttattagcagcatgagaaaggacgaatgcagcttccaagcaaggtaggcctgctatatcaaggtcagagcccttgttgggaaaggatgggaccctgacatgtgaaacagggacatctgagttgatgcccctgaaaatcttcgatttctagattctcttgaatgctctgagcctgcagaagtgcccttccctggctcctactcgaaaatgccaaggctttttcccacagtcaagaaggaacccacctcccctcctggccatgaaatgtaaactagggttaagtcacagtataactcagggacatgatgggcctaataaaggagaaaagggattataccccaaagtagctgcaagacctaaccagcatacaccagcaagaagctgggaaggacatgtggactgaatgctgagggtgcttgatcaaggtggccagaacatgagattggatgagggagactgtgttgacttgggagcattctatcccataatacacaatttaacatcctagcaaaggctcccagagattctatgaactcactcttagaagcatgaaaaaaataaaggcctgctgggtgcagtggctcacatctgtaatcccagcactttgggaggctgaggcaggtggatcacctgaagtcaggagtttgagaccagcctggccaacgtggtgaaaccctgtctctactaaaaaaaaaaatacaaaaacaagccgggcatggtggcaaatgcctgtaatcccagctactggtggactgaggcagaaggactgcttgaacctgggaggcggaggttgcagtgagctgagatcgtgccactgcactacagcctggacaacagagtgagactgttgtctcaaaaaataaataaataaataaataaaaataaaaaataaagcccgtagtaaatggggtagaaatgccatcattgccctggtggacagtagaggaaggtgtcacaaggggaagggcagtaaccttctggagtagacacactgcaaaaagtccagaaaacccacctgatggtaatgttccatagggaggcgcagaggacaaccattctgaggacatcagaatgtactgatgagaggggacaccagcatcaataaaaaattcaggccgggtagggtggctcacacctgtaatctcaatactttgggaggccaaggcagaaggatcactagagcccaggagtgagagaccaacctgggcaatgtggcaaaaccccgtttctgcaaaaaaatgcaaaaattaaccaggcttggtggcacacacctgtagtcccagccacctgggaggctgaggcagggcctggaagttcaaggctgcaatgagccaagatcacacctactgcactccagcctgggggaaagagcaagaacctgtgtcataagtaaataaataaataaataaataaataaataaataaataaataaaatgtagacttagctgtctctgtaggccaatactggattgaagaagttcttatagaataaggcctactgatagcagagagaatgataggacccaaaacaacagaggtggtttgggccttaactatctgtgtgggtatgggggtcatttttcctggctgcaaccttgtgcttatctctaggggatcccgaagccatagtgctgagtcttgatccatccagaatggaatttgcaaatcattgattagtgtagttctgtactgagtccaaaaactactacagatattctgtaccaacttgtcaccattgttaagtgaaaatgtacagattgattattttcacctggtttctgttagattatcttagttgagcctgatcatgcaagctggttttggctatgtgaccagaaggatattaaaaaccatcttagatgagattgtgacttccctgagaccaatgcattttgcatgtatcttggtggtttatcatcctaagatcaagtacatcctatgtacctgaaaggaagatgcgcctggatgtcggacagctccctggtatttgcctttatcctgctatattattttctttagttttatgaaagccctatgtgagtatactttcaggagtcttcttgtgattccttccactacctgaggaggcacaactggggcaccatcagaagccaggcaagtgcactgattgtagtaactggccaggttggagtggcagcccaggagcctggtctacagagagttatggaagtgggtgatagagcacagagtccatgggggcaaaagagatgggagaccaggccaggcgcggtggctcatgcctgtaataccagcactttgggaggctgaggtgggcagatcacaaggtcaggagtttgagaccagcctagtcaatatggtgaaaccccatctctaccaaaaatacaaaaattagccgggcgtggtggcgggcacctgaagggccagctactcacgaggctgaggcaggagaatcatttgaacctgggaggcggaggttgcagtgagccgaccttgtgccactgcaatccagcctgggtgacagagcaagactctgtctcacaaaaaaacaaaaacaaacaaagaaaacaaaaagagatgggagacccaaaagggtactgctcaacctgcaccattagaataaatcatgatgaatgatcaggaagctaaggcagacaccccagtaaaaagtcacacgttgcccagttttcagacctgaggtagttttcagatccagaaccaattgattgaaaagaagcctggctcctcattaagaaggactgtgcagcaccatggcaaatgggaaaagaggaatacccaaatattctgaggactgtaagacacagtgtctgagctggcattgataaccagaggcctgaagtatcatcatggccttcgttagagaggaagtatttggagacctggtaataaattgagtcccggccaaggtctggctgacagtgagtccactgggtccacggaccccatccagtcattatctcccacatgaaaaatcgagatagaaatatttgacccggatgtgatctacaggtggagtgacagcccaggagcctgatctacagagagttgtggaagtgggtaatagagcacagagtccctgagggcaaagagagatgggagactgaaaacgggactgctcaacctgcaccatcaaaagaaaccaaggatgaattcttggagtgtgtgtgatcctctcctatatacacactgagctggcttgtggagtaagagctatagtagttgagaagcctccaagcagtccccttccaacccaggtcaacataataaatcagaaacaatattgcatcccgggatgaaagagttgagaaggttaatgccacccttggggaactgaaggatgcaagggtaatagttaccttcatgtctctagtatggttcatgtaggagaagagggattctatggaatgacactagactactgcaaagtaaatggagtagtagctgccattgcacctaccatgccagatctttgctagaatagatgaacagtttcagacacatagtgtgtggccattgttagagaattcattcttttctcttcctatctgaaaagaggattcaaaatagtccacattcacatggaatggacaacagtatacatttacaactcccccccatacattaactttcccaccgtctgtcacagtctggtctgtgccagtttttcaatgtattgactctcatctccaagtcaccctgcattacctgctttgtgataatggagctagacactttaagcatttctcccttgtggtgagcatcaggttaagctttgttgtagatgccctagagcgacaaagcagaaagaaaaggctttccagctgggtgtggtggctcatgcctgtaatcctagcactttgggaggctgaggcaggtggatcacctgaggtcaggagttcaagacctgcctggccaacatggtgaaaccttgtctctactaaaaatacaaaaattagctgggtatggtggcatgcacctgtaatcccagctactcaggaggctgaggcaggagaatcacttgaatccaggaggtggagcttgcagtgagccaagatcacaccactgcactccagcctgggcaatagagtgagactccatctcaaaaagaaaaaaaaaaaaagaaaaaagaaaaagaaaagaaaaggcttttcttgctgtttctgtcatgcttgggttttgcttcttcttgcctctactgtatggttgccagaggcacaggagagagggcctctgccccagctccatacgcagagtgtggggtccttggtgagctcacagcattggccccagctaggtgcccagctcactgtggctctcccaatgaggttacatgcacccaggatttacactgctctgctagggatcaagctcctgacacaccgactcctcctgtgcatccaccttccagcactggctcacctgtataatgcagggttgcttcctggggcccccctaatacagatatggcacacctcagtccttactctgcctcagtgcaagcaggctcctgatgccccgactacctctgtgcactgatcaccagcacccttataattcctcctctgttggaatcactgcttctttatcttaaacttttcctgttcaaattactctgtggcttctgtccctgttttggaccctgaatgatacaaagagatctgggccatctggacattttgcagaacgtggccttgatctgttatgtgaatgacatcctgctaaatgggccaaaggagcaaaaagtggctagcatgttaaaggcattggtaggttacacacactccagagggtgggagatgaatactacagatatttaggagcctgcacaccgggaacaattttaggggtcctgtggacaggagcatgtcaggacagtccatccaaagtcaaaatcaaatgattgcatctcgtatttcccaccatgaagaaggaagcacaaaacttagacattctctttggcttctggagatagcatattccacacctgggaataccacttgaccaataataaccagataacctgaaaggctgtcagcttgaattggggcccagaacaggaaagggctctgcagtaggtccaggcagcagtgcaagtagccttgcgattgagccatcaacccagcagactctatagtacaaaaggcatgatgatgaagaagatgctgtgtagagcttgtagcatgccccagtgggagagtcacagtgcaggctcccagggttctggagcgaggccatgccatctgcagtggaggatcatatgcctttggcaaaacaactcctggcacgctgctgtgccctggtagaaatggagcacctgatggtgggataccaactgatcttgcacctggaactgcccttcattaactgggttacatcagatgagttaaattataaggttaggcaagcccagcaacaatccattgttagatggaagttgtacatctgggattgagcatggacaggacctgggccacaagcaagctgcatgtgcagtctgttcaggcccccaagccacccaccggagttgcaccagcaccctccctccacacacacctaaggttataggaggggtccttctgaccagctgatggaagaggaaagagcaagtttggtttgtgaatggcttggcttggtttgtgggtttaggctgaaaacaacaggaaatgtactacaatctcactcaagggtggccttgaaagagagtggcaaggcaaaaccctcctaatgggcagggatttgggtggtgtagctgttcacccattttgtatggacagagcagagctcaaggtgagaatatatacaaacttctgggtagtggtgaatggcctggccagctgatcatagatgtggaaagggaacaattagaagactggagacaaggagatctgggcagaggcatgtggatgacataggcaagagtacacaaagggccaggcctggtggctggtacctgtaatcccagcactttgggaggccgaggcaggtggatcacctgaggtcaggagttcgagaacagcctggccaacatggtgaaacctcgtatctagtaaaaatacaaaaattagctgggtgtggaggcccgtgcctataatcccagctacttgggaggctgaggcaggacaatggtttgaacctgggaggcagaggttgcagtgagccaagatcgtgtcactgcactccagcctgggcgaaagagcaactctgtctccagaaaaaaaaaaaaaaaaaaagagcacacaaagcttgatctttgtgccacgtgttaatgcccactagagatgacctactatggaagagacagtggacaaccaagtagacaagaggacgagagtgggggatgccagttggcctctgccatgggccgtcccaatgctgacctgatggacatacgaatgaagcagcagtggtgacagaaatggatgctatgtgcaggatgggtcccatgtaccaaggctaccgctgccaccaaatgtccaacctcccgcaacagagaccagtgctaagtcttcagaatggaccatttctcaaaaagacgaaccggcaggttgactttgttggggtcccttcacacataaggcccagtaatttgttctgacaggagtagacccatgatctgagtacggatttgcctttcctgccttcggtgcctaagccagtatccttatctgagggcttacagtgtgtttgattctccagtacagggtcccacatagcgtcacatgagaccaagggaaatgctttacagcaggggaggggtgggactgaacccacagccacgggatcctctagttaattcataggcttctccatctagaagcagctgccctgagtttggaaggcacacctgaaccatcaattctttacaagggtaggagacgtactccaggatgcaggacagaccttgaatcaaagacctttttacagccttgtggtagaaaaatggccctaaagaagttcatgcccaagtccctgggacctgtgaatgtggtttttgtttgtttgtttgtgtttgtgtgtttgtttgtttgttttgagatggggtctcgctctgttgcccaggctggagtgcagtggcgcaatctcggctcactgcagcctccgcctcctgggctcaaggaattctcctgcctcagcctcccgagtagctgggactgcaggtgcacaccaccacacctgactaatttttgtgtttttagtagaaacggggtttcaccatgtgggacctgtgaatgtgttagtgtacatagcaaaaggaactttgcaaatgtgatgaaaagtagggaccttgagatgacgggtggatccccgattatccaggtgagcccagtatagtcacaagagtccttaaaagtggaaataatttcctggctgcagtaagagtgagagatgtgacacggaagaaggatcagagagatgtaacgttgctggcttttttttttttttttttttgagacagggtctccctctgtcacccaggctggagtgcagtagcgacatctcagctcactgcaacctccatctcctgggttcaagcgattctcctgcctcagcctcccgagtagctgagatcacaggcgtgtgccaccacgcccagctaattttattttatttatttttttgtacttttagtagagacagggttttgccatgttggccaggctggtctgaaactcctggcctcaagtgagccaccagcctcagtctcccaaagtgctgggattacaggcgtgagccacagcacccggccatgatgttgctggctttgaagatgaggaagaggccaagtgctaaggagtccacctactctagaaactggaagaggtgaagcaggggaggggtgggagtgaacccacagccacgggatccactggttatatcataggcttctccatctagaagcggctaccctgagagaattggtcaagcacagcttggaaggcacagccgaaccatcaattctttacaagggtaaaaattctttacaaaggatgcaggatcctctgctagagcctccctgtcgacaccttgattttagcctagtgaggtctttgttggacttctgacctacagaactctaagatgatatatttgcattgttttaagccactgagtttgtagcacagccatacaccagacaaccacaatggactgcatttatgacggcagtcccataagattacactggagctgaaaaatccctactgcctagtgacttcatagcggtgatgatgttgtagcacaatacattactcctgtgtttgtgatgatgccggtatgcaaacctacttgctggctgttgtataaaagtctagcacatacaattatgtacagtacataatattggataatgatggctgggtgcaatggctcatacctgtaatcccagcactctgggaggccaaggtgggtggatcacctgaggtcaggagtttgagaccaccctggccaacatggtgaaaccctgtttctactaaaaatacaaaaattagccgggcgtgatagcgggcacctgcaattgcagctactcaggaggctgaggcaggagaatcacttgaacgtgggaggtggaggttgcagtgagtggagatcgcgccattgcactccaacctgggcaacagagtgagactccatttgaaaaaaaaaattggataatgataataaacgactgtgttactggtttgggtacttactatactatattttttattattattctagagtgtacttcttctacttattaaaaaaaaaagttaactgtgaaacagcctcaggcagggccttcgtgagggattccagaagaaggcattgttataataggagatgacggcttcatgcatgttactgcccctgaagaccttccagtgggacaagatggggaggtagaagatggtggtgatactgatgaccttgaccctttgtaggactaggctaatatgtgtgttgatgtcttagtttttaccaagaaaagtgtaacaagtaaaaaaaaacaaaatagaaaaaagcttataggataaggacataaagaaagaatttttgtacagctgaaaacgtatttgtgttttaagctaaacgttactacaaaagtcaaaaagttttaaaaatttaagagtttataaagcaaaacaagttacagtgtgctaagtttaatttattgttgaagaaaaataattttaataactttaatgtggactaaatgtacagtgtttataaagttgatagcaggctgggagctgtggctcacacctgtaatcccagcactttgggaggctgaggcgggtgggtcacctgaggtcaggagttcaagaccagcctgaccaccatggagaaacccaatctctactaaaaatacaaaaaattatctgggtgtggtggtgcatgcctgtaatcccagctacttgggaagctgaggtaggagaatctcttgaacccgggaggcggaggttgcggtgagccaagatcctgccattgcactccagcctgggcaagaagagtgaaattccatctcaaaaaaaaaaaaaagtcaacagtagtgtacagtgatgtcctaggcctttacattcactcaccactcactcactgactcacccagagcaacttccagtcctacaagttccattcatggtaagtgtcctaaacagaggtactgtttttcaactttttattttattttattttattttattttattttttgagatagagtctcactctgtctgccagggtagagtgcagtggtgcaatcatggctcactgcaaccttgaactcctgggctcaagcaatcctcctacctcagcctcctgggtagctgggattgcaggtgcatgccaccatacctggctaatttttgattttttgtagagacaaggtctcgctgtgttgcctaggctgatctcgaactcctgggctcaagtgatcttcctggctgggcctcccaaagtgctgggattactggcatgagccgctgcgtttatactattttatactgcttttactatacatttactatgtttagacacatttagatacacaaatacttactattgtgttaccgttgctgcagtactcagtacagtaacatgctgtacaggtttgtagcctaggagcaaaaggctataccatatagcccaggcatttagtagtacctagtgccatctaggctgtgtaagtgcactctatgacgctcacatgaagaaatcacctaatgaagcatttcttagaacgtatcccagtctttaattggttccagcagcgatagaaaacaaaccccgacaggaaaaaatacatgggtttgggaaccaagggtgaaaactgaagtggccgcatttaccgccacatcattacaactctaagctcttcagggttagaggtcctggtctcccaaagggaaaacttccaccaggggacacagcacagttttattgaactatcagctgtggctgctgcctgagcccttctgcttccttatgtccagacatcagcaggcaaaaagaaaagttaccatcttggaagggataattaactttagtcattagcaggaagtagggctgcaattatacagtgcagcagggaggaacacgtttgttacctgggcaccggttgctactcccttgcccaaccatgattgctaagggacagcaatttctcttatttctctcttacatagaagtccagaagtaggagtcaaaggctgttttagaagctccaagaccatcagggagctcttatccagctgtatggtttctctaccatccttagcatgctgcctcatagtccaaaacagctgcctgagctccagtcatcacatctgcattctagcccccagaaagaaggaagagaacaactttactttaaggacatttcctggaagtcacatatgacatccttgctaacatcctattggccagaatttagacattaccacacaaagttgccatcgaggctggaaaatatatttttattacagggacttgtgtccagctaaataccagctataggaggagaaggggacattggggaacagccacaataatgctaagaggccatcatacttggtaattcttcccagttatcatgagcagcttactctttgaaatagtttcatgaaaacactttcataaaaaggcattttctactgaaacagatgacagccttcaagactaacattcctttattttctattgtaaattcagacaccaaattcagggcatatttagacagactaacatgcatttgttctgcctaattgtctgctaaacagggtgctttgatgagacacgacagccctctctcaaagccaatattgggaaatatcaatataaaatacagctaaattagaaagagagtattaacattacagacaaatacttcactctacaaaatgaaccattaccaggttcccctaagtgtcagttctcctgttgcaaatgaaatgtgagttggtttatgcggacattttcaacatcttaattatataacgtgaggcagatccatgctttcatttccctggacttacctctcactgattattgtttagactcacatgttcctttctgtgtgtctatttcctggatgcctccttcccacctgcccttctctccattatcctgccagtgacaactgccccttgtccaggtttcccgggttctagcaagaagaaggtaactgcagatgagggtttgagttacctatgactcagcagcatgactctatcgtcaaaaaagcaaggatgatcctggggtccagaaatagggacaaggcatcaccgatccatgagctcatcttctcatcatatttggctttgtgcagacactctggatatcctggcatagcggaatgacagatcatttgaagagtgccccaaatacaaaacttgaaaagggtgagcagaaatccctttcacagaaaccatgctatcagttgtgaaatcctactttatggaagatataatcggaaatttaaaagttaccaaaatattaagatctgagtcaaatctcatgacctatagtttggcattgatacttattttatttagatacagggtctcactctgtcacccaggctgaagtgtaatactgtgatcagacctcactgtagcctcgatgttctgggttcaagcgatcctcccgactcagccatccgagtagctgggactacaagcgcgcaccaccatgtctagataatttgtgtgtgtgtgtggagacaggttctcactgtgttgcccaggctggtcttgaactcctgggttcaagccatcctcctgcctcagactcccaagtagctgggactacaggcgtaagccactgttcctggcttgggcttcattttttacaagctgttctttattcggaataaaaagaattagcatctctttttctaagtaatactaagtacaaccactcactgttactagattagtgttctggtttttcttttgaaatcggagaggcaggctattcatctttccatccattcaacaggtgtttgagtgtccctgtacaccgtgaatacaatgaagactaagatcttaagttgcttacaatgtagtgatttgcaggtgagttatatatatatatagtttgttggttggttttttgagacggagtcttgctctgtcgtccaggctggagtgcagtggcgcgatctcagctcactgcaagctccgcctcccgggttcacgccattctcctgcctcagcctcccaagtagctgggactacaggtgcctgccaccacgcccagctaatgttttgtattttttagtagagacagggtgtcaccatgttagccaggatggtctcaatctcctgacctcatgagccgcccgccttggcctcccaaagtcctgggattacaagctgagccaccgcacccggcctattaatttatattggtctagctaggcatggtggctcacacctgtaatcctagcactttgggaggccgaggcgggtggatcacctgaggtcaggagttcgagaccagcctgagaacatggtgaaaccctgtctcttctaaaaatacaaaaattagcgaagcgtggtggtacatgcctgtaatcccagctacttgggaggctgaggcaggagaatcgcttgaatccgggaggcggaggttggagtgagccgagatagtgcctgcactccagcctggatgacagcaagactctgtctcaaaaaataagaaaataaataaatatttatattgatttcaagaatttggggataactactctgaaaaggctacatactgtaggattcaacgatatgaaattctggaaaaggcaaaactatggaggcagcaaaaagatcagtggctgccaggggttagcagggagggagggatggataggcagagcacgggggatttttagggcaatgagactgttctgtatgctagtataatcgtggatacatgtgattgtatatttgtcaaaacctatagagtatacaacagcaagagtgagccctaatataaactatggactttgggttataatgacatgtcaaggtaggttcatggattgtaagaaatgtaccactctggtgggggatgtcgatagtgcgggaggctgtatagagcagagggtatacggggattttctgtactttctgctcaattttgctgagaacttaaaattgctctaaaaagtagtctatgttttaaattaaaaatgaaaaaatagattggcatggtggctcacgcctgtaatcccagcactttgggagcctgagatggcagacagcttgagctcagaagttcgagaccagcctgtgcaacatggcaaaaccctgtctctgcaaaaaatacaagacttatctggacttgggggcatggtggtcccagctactcaggaggctgaagtgggaggatcacctgagtccaggaggtcgaggctgcagtgagccaggatcatgccactgcactccagcctgagcaacagagtgagaatctgtctcaaaaaaaaaaaaaaattaaaacataactggggaaatccacaaaaattgggggttagataccctctgcgaagcttcagatgatcactttcaactattgtgt

SEQ ID NO: 6 - DNA, 3641 bp, Homo sapiens
agtctgggatccttgagtcctactcagccccagcggaggtgaaggacgtccttccccaggagccggtgagaagcgcagtcgggggcacggggatgagctcaggggcctctagaaagagctgggaccctgggaacccctggcctccaggtagtctcaggagagctactcggggtcgggcttggggagaggaggagcgggggtgaggcaagcagcaggggactggacctgggaagggctgggcagcagagacgacccgacccgctagaaggtggggtggggagagcagctggactgggatgtaagccatagcaggactccacgagttgtcactatcatttatcgagcacctactgggtgtccccagtgtcctcagatctccataactggggagccaggggcagcgacacggtagctagccgtcgattggagaactttaaaatgaggactgaattagctcataaatggaacacggcgcttaactgtgaggttggagcttagaatgtgaagggagaatgaggaatgcgagactgggactgagatggaaccggcggtggggagggggtggggggatggaatttgaaccccgggagaggaagatggaattttctatggaggccgacctggggatggggagataagagaagaccaggagggagttaaatagggaatgggttgggggcggcttggtaaatgtgctgggattaggctgttgcagataatgcaacaaggcttggaaggctaacctggggtgaggccgggttggggccgggctgggggtgggaggagtcctcactggcggttgattgacagtttctccttccccagactggccaatcacaggcaggaagatgaaggttctgtgggctgcgttgctggtcacattcctggcaggtatgggggcggggcttgctcggttccccccgctcctccccctctcatcctcacctcaacctcctggccccattcaggcagaccctgggccccctcttctgaggcttctgtgctgcttcctggctctgaacagcgatttgacgctctctgggcctcggtttcccccatccttgagataggagttagaagttgttttgttgttgttgtttgttgttgttgttttgtttttttgagatgaagtctcgctctgtcgcccaggctggagtgcagtggcgggatctcggctcactgcaagctccgcctcccaggtccacgccattctcctgcctcagcctcccaagtagctgggactacaggcacatgccaccacacccgactaacttttttgtattttcagtagagacggggtttcaccatgttggccaggctggtctggaactcctgacctcaggtgatctgcccgtttcgatctcccaaagtgctgggattacaggcgtgagccaccgcacctggctgggagttagaggtttctaatgcattgcaggcagatagtgaataccagacacggggcagctgtgatctttattctccatcacccccacacagccctgcctggggcacacaaggacactcaatacatgcttttccgctgggcgcggtggctcacccctgtaatcccagcactttgggaggccaaggtgggaggatcacttgagcccaggagttcaacaccagcctgggcaacatagtgagaccctgtctctactaaaaatacaaaaattagccaggcatggtgccacacacctgtgctctcagctactcaggaggctgaggcaggaggatcgcttgagcccagaaggtcaaggttgcagtgaaccatgttcaggccgctgcactccagcctgggtgacagagcaagaccctgtttataaatacataatgctttccaagtgattaaaccgactcccccctcaccctgcccaccatggctccaaagaagcatttgtggagcaccttctgtgtgcccctaggtactagatgcctggacggggtcagaaggaccctgacccaccttgaacttgttccacacaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgaggtgagtgtccccatcctggcccttgaccctcctggtgggcggctatacctccccaggtccaggtttcattctgcccctgtcgctaagtcttggggggcctgggtctctgctggttctagcttcctcttcccatttctgactcctggctttagctctctggaattctctctctcagctttgtctctctctcttcccttctgactcagtctctcacactcgtcctggctctgtctctgtccttccctagctcttttatatagagacagagagatggggtctcactgtgttgcccaggctggtcttgaacttctgggctcaagcgatcctcccgcctcggcctcccaaagtgctgggattagaggcatgagccaccttgcccggcctcctagctccttcttcgtctctgcctctgccctctgcatctgctctctgcatctgtctctgtctccttctctcggcctctgccccgttccttctctccctcttgggtctctctggctcatccccatctcgcccgccccatcccagcccttctccccgcctcccactgtgcgacaccctcccgccctctcggccgcagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagtgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgcatctgctggcctccccctgtgattt

SEQ ID NO: 7 - DNA, 3641 bp, Homo sapiens
agtctgggatccttgagtcctactcagccccagcggaggtgaaggacgtccttccccaggagccggtgagaagcgcagtcgggggcacggggatgagctcaggggcctctagaaagagctgggaccctgggaacccctggcctccaggtagtctcaggagagctactcggggtcgggcttggggagaggaggagcgggggtgaggcaagcagcaggggactggacctgggaagggctgggcagcagagacgacccgacccgctagaaggtggggtggggagagcagctggactgggatgtaagccatagcaggactccacgagttgtcactatcatttatcgagcacctactgggtgtccccagtgtcctcagatctccataactggggagccaggggcagcgacacggtagctagccgtcgattggagaactttaaaatgaggactgaattagctcataaatggaacacggcgcttaactgtgaggttggagcttagaatgtgaagggagaatgaggaatgcgagactgggactgagatggaaccggcggtggggagggggtggggggatggaatttgaaccccgggagaggaagatggaattttctatggaggccgacctggggatggggagataagagaagaccaggagggagttaaatagggaatgggttgggggcggcttggtaaatgtgctgggattaggctgttgcagataatgcaacaaggcttggaaggctaacctggggtgaggccgggttggggccgggctgggggtgggaggagtcctcactggcggttgattgacagtttctccttccccagactggccaatcacaggcaggaagatgaaggttctgtgggctgcgttgctggtcacattcctggcaggtatgggggcggggcttgctcggttccccccgctcctccccctctcatcctcacctcaacctcctggccccattcaggcagaccctgggccccctcttctgaggcttctgtgctgcttcctggctctgaacagcgatttgacgctctctgggcctcggtttcccccatccttgagataggagttagaagttgttttgttgttgttgtttgttgttgttgttttgtttttttgagatgaagtctcgctctgtcgcccaggctggagtgcagtggcgggatctcggctcactgcaagctccgcctcccaggtccacgccattctcctgcctcagcctcccaagtagctgggactacaggcacatgccaccacacccgactaacttttttgtattttcagtagagacggggtttcaccatgttggccaggctggtctggaactcctgacctcaggtgatctgcccgtttcgatctcccaaagtgctgggattacaggcgtgagccaccgcacctggctgggagttagaggtttctaatgcattgcaggcagatagtgaataccagacacggggcagctgtgatctttattctccatcacccccacacagccctgcctggggcacacaaggacactcaatacatgcttttccgctgggcgcggtggctcacccctgtaatcccagcactttgggaggccaaggtgggaggatcacttgagcccaggagttcaacaccagcctgggcaacatagtgagaccctgtctctactaaaaatacaaaaattagccaggcatggtgccacacacctgtgctctcagctactcaggaggctgaggcaggaggatcgcttgagcccagaaggtcaaggttgcagtgaaccatgttcaggccgctgcactccagcctgggtgacagagcaagaccctgtttataaatacataatgctttccaagtgattaaaccgactcccccctcaccctgcccaccatggctccaaagaagcatttgtggagcaccttctgtgtgcccctaggtactagatgcctggacggggtcagaaggaccctgacccaccttgaacttgttccacacaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgaggtgagtgtccccatcctggcccttgaccctcctggtgggcggctatacctccccaggtccaggtttcattctgcccctgtcgctaagtcttggggggcctgggtctctgctggttctagcttcctcttcccatttctgactcctggctttagctctctggaattctctctctcagctttgtctctctctcttcccttctgactcagtctctcacactcgtcctggctctgtctctgtccttccctagctcttttatatagagacagagagatggggtctcactgtgttgcccaggctggtcttgaacttctgggctcaagcgatcctcccgcctcggcctcccaaagtgctgggattagaggcatgagccaccttgcccggcctcctagctccttcttcgtctctgcctctgccctctgcatctgctctctgcatctgtctctgtctccttctctcggcctctgccccgttccttctctccctcttgggtctctctggctcatccccatctcgcccgccccatcccagcccttctccccgcctcccactgtgcgacaccctcccgccctctcggccgcagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgcatctgctggcctccccctgtgattt

SEQ ID NO: 8 - DNA, 3641 bp, Homo sapiens
agtctgggatccttgagtcctactcagccccagcggaggtgaaggacgtccttccccaggagccggtgagaagcgcagtcgggggcacggggatgagctcaggggcctctagaaagagctgggaccctgggaacccctggcctccaggtagtctcaggagagctactcggggtcgggcttggggagaggaggagcgggggtgaggcaagcagcaggggactggacctgggaagggctgggcagcagagacgacccgacccgctagaaggtggggtggggagagcagctggactgggatgtaagccatagcaggactccacgagttgtcactatcatttatcgagcacctactgggtgtccccagtgtcctcagatctccataactggggagccaggggcagcgacacggtagctagccgtcgattggagaactttaaaatgaggactgaattagctcataaatggaacacggcgcttaactgtgaggttggagcttagaatgtgaagggagaatgaggaatgcgagactgggactgagatggaaccggcggtggggagggggtggggggatggaatttgaaccccgggagaggaagatggaattttctatggaggccgacctggggatggggagataagagaagaccaggagggagttaaatagggaatgggttgggggcggcttggtaaatgtgctgggattaggctgttgcagataatgcaacaaggcttggaaggctaacctggggtgaggccgggttggggccgggctgggggtgggaggagtcctcactggcggttgattgacagtttctccttccccagactggccaatcacaggcaggaagatgaaggttctgtgggctgcgttgctggtcacattcctggcaggtatgggggcggggcttgctcggttccccccgctcctccccctctcatcctcacctcaacctcctggccccattcaggcagaccctgggccccctcttctgaggcttctgtgctgcttcctggctctgaacagcgatttgacgctctctgggcctcggtttcccccatccttgagataggagttagaagttgttttgttgttgttgtttgttgttgttgttttgtttttttgagatgaagtctcgctctgtcgcccaggctggagtgcagtggcgggatctcggctcactgcaagctccgcctcccaggtccacgccattctcctgcctcagcctcccaagtagctgggactacaggcacatgccaccacacccgactaacttttttgtattttcagtagagacggggtttcaccatgttggccaggctggtctggaactcctgacctcaggtgatctgcccgtttcgatctcccaaagtgctgggattacaggcgtgagccaccgcacctggctgggagttagaggtttctaatgcattgcaggcagatagtgaataccagacacggggcagctgtgatctttattctccatcacccccacacagccctgcctggggcacacaaggacactcaatacatgcttttccgctgggcgcggtggctcacccctgtaatcccagcactttgggaggccaaggtgggaggatcacttgagcccaggagttcaacaccagcctgggcaacatagtgagaccctgtctctactaaaaatacaaaaattagccaggcatggtgccacacacctgtgctctcagctactcaggaggctgaggcaggaggatcgcttgagcccagaaggtcaaggttgcagtgaaccatgttcaggccgctgcactccagcctgggtgacagagcaagaccctgtttataaatacataatgctttccaagtgattaaaccgactcccccctcaccctgcccaccatggctccaaagaagcatttgtggagcaccttctgtgtgcccctaggtactagatgcctggacggggtcagaaggaccctgacccaccttgaacttgttccacacaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgaggtgagtgtccccatcctggcccttgaccctcctggtgggcggctatacctccccaggtccaggtttcattctgcccctgtcgctaagtcttggggggcctgggtctctgctggttctagcttcctcttcccatttctgactcctggctttagctctctggaattctctctctcagctttgtctctctctcttcccttctgactcagtctctcacactcgtcctggctctgtctctgtccttccctagctcttttatatagagacagagagatggggtctcactgtgttgcccaggctggtcttgaacttctgggctcaagcgatcctcccgcctcggcctcccaaagtgctgggattagaggcatgagccaccttgcccggcctcctagctccttcttcgtctctgcctctgccctctgcatctgctctctgcatctgtctctgtctccttctctcggcctctgccccgttccttctctccctcttgggtctctctggctcatccccatctcgcccgccccatcccagcccttctccccgcctcccactgtgcgacaccctcccgccctctcggccgcagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgcgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagtgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgcatctgctggcctccccctgtgattt